(19) United States
(12) Patent Application Publication
(10) Pub. No.: US 2010/0216798 A1
(43) Pub. Date: Aug. 26, 2010

(54) FUSED HETEROCYCLES AS LCK INHIBITORS

(75) Inventors: Kazuo Nakai, Tokyo (JP); Fumie Takahashi, Tokyo (JP); Kazuya Fujita, Tokyo (JP); Yoshihiro Kozuki, Tokyo (JP); Koichiro Mukoyoshi, Tokyo (JP); Masamichi Inami, Tokyo (JP); Norio Asai, Tokyo (JP)

Correspondence Address: SUGHRUE-265550, 2100 PENNSYLVANIA AVE. NW, WASHINGTON, DC, 20037-3213, US

(73) Assignee: ASTELLAS PHARMA INC, Tokyo (JP)

(21) Appl. No.: 11/993,229

(22) PCT Filed: Jul. 27, 2006
(86) PCT No.: PCT/JP2006/315346

§ 371 Date: Dec. 20, 2007

Related U.S. Application Data
(60) Provisional application No. 60/703,497, filed on Jul. 29, 2005.

(51) Int. Cl. A61K 31/5025; C07D 487/04; A61K 31/519; A61P 37/00; A61P 11/06; A61P 37/06

(52) U.S. Cl. 514/248; 544/281; 544/236; 514/259.31

(57) ABSTRACT

There is provided fused heterocycles of imidazopyridazine or pyrazolopyrimidine derivative represented by the formula (I), which have excellent Lck inhibitory activity and are useful for a medicament particularly an immunosuppressive agent.

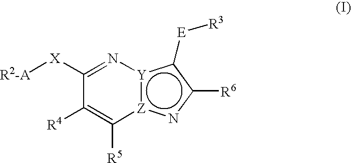

[wherein one of Y and Z is C atom, and the other is N atom; —X— is —N(R¹)— or the like, —R¹ represents hydrogen or the like, -A- represents bond or the like,
—R² is cycloalkyl, aryl or the like, -E- is bond or the like, —R³ is aryl, aromatic heterocycle or the like, —R⁴, —R⁵ and —R⁶ are the same or different, each being hydrogen or the like.]

### TECHNICAL FIELD

The present invention relates to a novel imidazopyridazine or pyrazolopyrimidine derivative and a pharmaceutically acceptable salt thereof, which is useful as a medicament particularly as an Lck inhibitor, and a pharmaceutical composition comprising the compound as an active ingredient.

### BACKGROUND ART

The compound that inhibits a signaling cascade of T-cell receptor (TCR) targeting to calcineulin is used widely in the transplantation area at this moment. Although these compounds show a strong immunosuppressive activity by inhibiting the TCR signal, these might have the problem of the side effect of renal toxicity and the like.

Lymphocyte protein tyrosine kinase (Lck) is one of members of Src kinase family, which is non-receptor type protein tyrosine kinase. Lck is located in initial step of the TCR signal transduction pathway, it phosphorylates and activates ZAP-70′, elevates intracellular Ca²⁺ concentration, and ultimately induces production of interleukin-2 and proliferation of T-cell. Additionally it is well known that Lck is essential for transduction of the TCR signal, which was showed by analysis of Lck knock-out mouse. Therefore Lck inhibitor is anticipated to have a strong immunosuppressive activity as well as calcineulin inhibitors.

Furthermore, since Lck is expressed only on T-cell, the effect of Lck inhibitor is limited to lymphocytic organ. Therefore there is low concern on side-effect like as renal toxicity by calcineulin inhibition, and it is hoped that Lck inhibitor may become immunosuppressive agent with less side-effect.

In view of these, it is thought that Lck inhibitors are useful for medicine for disorder that participates in T-cell, for example, autoimmune disease like as psoriasis, atopic dermatitis, rheumatoid arthritis, systemic lupus erythematosus, nephrotic syndrome and the like, suppression of immunological rejection of graft-versus-host disease, organ transplant or the like.

Hitherto, as protein tyrosine kinase inhibitor, Src inhibitor or Lck inhibitor having the chemical structure of fused skeleton of an aromatic 5-membered heterocycle and an aromatic 6-membered heterocycle, pyrazolo[3,4-d]pyrimidine derivatives are disclosed in International Publication WO 02/76986 and WO 02/80926, imidazo[1,5-a]pyrazine derivative in Japanese laid-open patent publication No. 2005-89352, Pyrrolo[2,3-d]pyrimidine derivatives in WO 00/17202, WO 00/17203 and WO 98/41525, pyrrolo[2,1-f][1,2,4]triazine derivatives in WO 02/79192, WO 2004/009601 and WO 2004/013145. However each of these publication does not mention or give suggestions on imidazo[1,2-b]pyridazine or pyrazolo[1,5-a]pyrimidine related to the present invention.

Meanwhile, pyrazolo[1,5-a]pyrimidine derivatives represented by the formula (A) as Src inhibitor and Lck inhibitor are disclosed in Japanese laid-open patent publication No. 2005-8581.

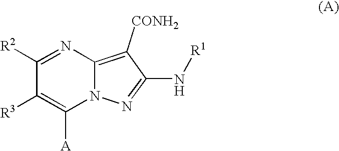

[wherein symbols areas defined in the above publication]

The compounds disclosed in the above publication are substituted with carboxamide at 3-position of pyrazolopyrimidine. However the compounds of the present invention are not substituted with carboxamide at 3-position at all.

Meanwhile, imidazo[1,2-b]pyridazine and pyrazolo[1,5-a]pyrimidine derivatives represented by the formula (B) as human protooncogene proviral insertion site in moloney murine leukemia virus kinase are disclosed in “J. Med. Chem. 48, pp 7604-7614, 2005”.

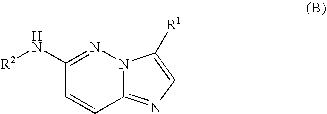

[wherein symbols are as defined in the above publication]

The compounds disclosed in the above publication are 3,6-disubstituted imidazo[1,2-b]pyridazine and 3,5-disubstituted pyrazolo[1,5-a]pyrimidine. However the compounds of the present invention do not include these compounds at all.

Given that situation, it is greatly desired to develop medicaments that have more excellent Lck inhibitory activity.

### DISCLOSURE OF THE INVENTION

The present inventors made extensive and intensive investigations with respect to compounds having Lck inhibitory activity, which are expected to be a safe immunosuppressive agent. As a result, it has been found that a novel imidazopyridazine or pyrazolopyrimidine derivative or a salt thereof of the present invention has an excellent Lck inhibitory activity, leading to accomplishment of the invention.

Accordingly, the present invention provides a fused heterocyclic compound of the following general formula (I) or a pharmaceutically acceptable salt thereof that is useful as an immunosuppressive agent.

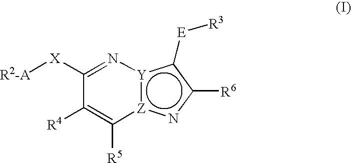

wherein
one of Y or Z is C atom, and the other is N atom.
—X— is bond, —N(R¹)—, —O—, —S—, —S(═O)—S(═O)₂—;
—R¹ is hydrogen or lower alkyl;
-A- is bond, lower alkylene or lower alkenylene, each of which may be substituted by one or more substituents selected from the group consisting of —OH and —NR¹¹R¹², wherein a methylene unit of -A- is optionally replaced by —O— or —C(═O)—;
—R¹¹ and —R¹² are the same or different, each being hydrogen or lower alkyl;
—R² is hydrogen, cycloalkyl, aryl, 5- or 6-membered non-aromatic heterocycle or 5- or 6-membered aromatic heterocycle, each of which may be substituted, or alternatively —R¹ and “-A-R²” taken with the adjacent nitrogen atom may form 5-, 6- or 7-membered cyclic amino, which may be substituted;
-E- represents bond, lower alkylene, lower alkenylene or lower alkynylene, wherein a methylene unit of -E- is optionally replaced by —O—, —(CO)O—, —NH—, —NHCO—, —NHSO₂— or —NH(CO)NH—;
—R³ is cycloalkyl, aryl, 5- or 6-membered non-aromatic heterocycle or 5- or 6-membered aromatic heterocycle, each of which may be substituted and may be fused with benzene; and
—R⁴, —R⁵ and —R⁶ are the same or different, each being hydrogen, halogen, lower alkyl, —O-lower alkyl or aryl.
provided that (i) when -A- is bond, —X— is NH, —R² is 4-tetrahydropyranyl and —R³ is 3-chlorophenyl, then Y is C atom and Z is N atom;
   (ii) when X is NH, —R² is cyclopropyl, 2-pyridyl, 3-pyridyl, 2-thienyl or 4-fluorophenyl and —R³ is 3-acetylphenyl, 3-chlorophenyl, 4-chlorophenyl, phenyl, 2-furyl or 2-thienyl, then A is bond.

or a pharmaceutically acceptable salt thereof.

Another one of the preferred embodiments of the present invention can be represented by the formula (I), wherein

one of Y and Z is C atom, and the other is N atom;
—X— is —N(R¹)—, —O—, or —S—;
—R¹ is hydrogen or lower alkyl;
-A- is bond or lower alkylene each of which may be substituted by one or more substituents selected from the group consisting of —OH and —NR¹¹R¹², wherein a methylene unit of -A- is optionally replaced by —O— or —C(═O)—;
—R¹¹ and —R¹² are the same or different, each being hydrogen or lower alkyl;
—R² is hydrogen, C₅₋₁₀ cycloalkyl, aryl, 5- or 6-membered non-aromatic heterocycle which contains one to three heteroatom(s) or 5- or 6-membered aromatic heterocycle which contains one heteroatom; each of which may be substituted with one to three substituent (s) selected from the group consisting of hydrogen, halogen, hydroxy, nitro, lower alkyl, —O-lower alkyl, —O-(6-membered cyclic amino), —CONH-lower alkyl, —C(O)NH-aryl, —S(O)-aryl, —C(O)O-lower alkyl, —C(O)OH, —C(O)NH—O-lower alkyl, —NR¹¹R¹², 6-membered non-aromatic heterocycle, and —O-(6-membered aromatic heterocycle), or alternatively —R¹ and “-A-R²” taken with the adjacent nitrogen atom may form 5-, 6- or 7-membered cyclic amino, which may be substituted;
-E- is bond, lower alkylene, lower alkenylene or lower alkynylene, wherein a methylene unit of -E- is optionally replaced by —NHSO₂— or —NH(CO)NH—;
—R³ is 5- or 6-membered non-aromatic heterocycle or 5- or 6-membered aromatic heterocycle which contains one to two nitrogen atom, which may be fused with benzene; each of which may be substituted with one to three substituent (s) selected from the group consisting of halogen, lower alkyl, lower alkyl having halogen, lower alkyl having hydroxyl, —OH, cyano, —O-lower alkyl, phenyl, —O-phenyl, —S-phenyl, —O-cycloalkyl, —C(O)O-lower alkyl, —C(O)NH₂, —NHCO-aryl, —NHC(O)O-lower alkyl and —NR¹¹R¹²; and
—R⁴, —R⁵ and —R⁶ are the same or different, each being hydrogen, halogen, lower, alkyl, —O— lower alkyl or aryl.

Another one of the more preferred embodiments of the present invention can be represented by the formula (I), wherein —X— in the compound represented by the formula (I) is preferably —NH— or —O—; -A- in the compound represented by the formula (I) is preferably bond or lower alkylene; —R² in the compound represented by the formula (I) is preferably hydrogen, cyclohexyl, phenyl, adamantyl, pyridinyl, piperidinyl or tetrahydropyranyl; each of which may be substituted with one or two substituent (s) selected from the group consisting of hydroxy, halogen, methyl and lower alkyloxy optionally substituted with halogen; -E- in the compound represented by the formula (I) is preferably bond; —R³ in the compound represented by the formula (I) is preferably pyridinyl which may be substituted with halogen; —R⁴ in the compound represented by the formula (I) is preferably hydrogen; —R⁵ in the compound represented by the formula (I) is preferably hydrogen or methyl; and —R⁶ in the compound represented by the formula (I) is preferably hydrogen.

The present invention also provides a pharmaceutical composition comprising one or more compounds represented by the formula (I) as an active ingredient, which is useful as an Lck inhibitor, especially as a medicament for disorder that participates in T-cell, for example, autoimmune disease like as psoriasis, atopic dermatitis, rheumatoid arthritis, systemic lupus erythematosus, nephrotic syndrome and the like, suppression of immunological rejection of graft-versus-host disease, organ transplant or the like.

The present invention will be explained in more detail herein below.

In the definition of the general formula for the compound in the present invention, The term “lower” used in the description is intended to include 1 to 6 carbon atom(s) unless otherwise indicated.

The term “lower alkyl” means a monovalent group of a straight or branched carbon chain such as methyl, ethyl, propyl, butyl, pentyl, hexyl, isopropyl, tert-butyl and the like.

The “lower alkylene” means a divalent group of alkane such as methylene, ethylene, trimethylene, tetramethylene, dimethylmethylene, dimethylethylene and the like. The “lower alkenylene” means a divalent group of alkene such as ethen-1,1-diyl, vinylene, propendiyl, butendiyl and the like. The “lower alkynylene” means a divalent group of alkyne such as ethyndiyl, propyndiyl, butyndiyl and the like.

The “cycloalkyl” means a non-aromatic carbon ring having 3 to 10 carbon, atoms, which may have partial unsaturation and may be fused or bridged. Its examples include cyclopropyl, cyclobutyl, cyclopentyl, cyclohexyl, cyclohepyl, cyclooctyl, cyclohexenyl, cyclooctadienyl, bornyl, norbornyl, adamantyl, 1,2,3,4-tetrahydronaphthyl and the like, of which preferred ones are ones having 5 to 10 carbon atoms.

The “aryl” means a mono- to tri-cyclic aromatic carbon ring having 6 to 14 carbon atoms, of which ones 6 to 10 carbon atoms, e.g. phenyl and naphthyl are preferred, and phenyl is more preferred.

The “5- or 6-membered non-aromatic heterocycle” means a monovalent group of a non-aromatic heterocycle having one or more hetero atoms selected from the group consisting of a nitrogen, an oxygen and a sulfur atom, which may be fused or bridged. Its examples include azetidinyl, pyrrolidinyl, imidazolinyl, imidazolidinyl, pyrazolidinyl, pyrazolinyl, dihydropyridinyl, piperidinyl, azepinyl, piperazinyl, homopiperazinyl, morpholinyl, thiomorpholinyl, tetrahydropyranyl, thiopyranyl, indolinyl, isoindolinyl, 8-azabicyo[3.2.1]octanyl, quinuclidinyl and the like.

The “5- or 6-membered aromatic heterocycle” means a monovalent group of an aromatic heterocycle having one or more hetero atoms selected from the group consisting of a nitrogen, an oxygen and a sulfur atom, which may be fused. Its examples include pyrrolyl, thiazolyl, oxazolyl, imidazolyl, isothiazolyl, isoxazolyl, pyrazolyl, thiadiazolyl, oxadiazolyl, triazolyl, tetrazolyl, pyridinyl, pyrazinyl, pyrimidinyl, pyridazinyl, pyridinonyl, indolyl, benzothiazolyl, quinolyl, isoquinolyl, 1H-isoindolyl-1,3(2H)-dione, 2-methyl-1,2,3,4-tetrahydroisoquinolyl and the like.

The “halogen” means chloro, bromo, iodo and fluoro.

As substituent groups that can be used for the term “optionally substituted” or “which may be substituted”, those commonly used as substituent groups for each group can be used, and each group may have one or more substituent groups.

As the substituent groups that can be used for “cycloalkyl, aryl, 5- or 6-membered non-aromatic heterocycle or 5- or 6-membered aromatic heterocycle, each of which may be substituted” in the definition of —R² and —R³, which may be one or more, preferably one to three and may be the same or different, the following groups (a) to (h) can be exemplified. Wherein, “R^{Z}” is a lower alkyl which may be substituted with one or more groups selected from the group consisting of —OH, —O-(lower alkyl), —C(═O)N(R^{Z})₂, amino which may be substituted with one or two lower alkyls, aryl, 5- or 6-membered aromatic heterocycle and halogen.

(a) halogen;
(b) —OH, —OR^{Z}, —O-aryl, —O-(protecting group), —O-(5- or 6-Membered aromatic heterocycle), —OCO—R^{Z}, oxo(═O);
(c) —SH, —SR^{Z}, —S-aryl, —SO—R^{Z}, —SO-aryl, —SO₂—R^{Z}, —SO₂-aryl, sulfamoyl which may be substituted with one or two R^{Z};
(d) amino which may be substituted with one or two groups selected from the group consisting of R^{Z} and aryl, —NHCO—R^{Z}, —NHCO-aryl, —NHCO— (5- or 6-membered aromatic heterocycle), —NHCO₂—R^{Z}, —NHCO₂-aryl, —NHCONH₂, —NHSO₂—R^{Z}, —NHSO₂-aryl, —NHSO₂NH₂, nitro;
(e) —CHO, —CO—R^{Z}, —CO₂H, —CO₂—R^{Z}, cyano, carbamoyl which may be substituted with one or two groups selected from the group consisting of R^{Z} and —O—R^{Z};
(f) aryl or cycloalkyl, each of which may be substituted with one or more groups selected from the group consisting of —OH, —O-(lower alkyl), amino which may be substituted with one or two lower alkyl, halogen and R^{Z}.
(g) 5- or 6-membered non-aromatic heterocycle or 5- or 6-membered aromatic heterocycle, each of which may be substituted with one or more groups selected from the group consisting of —OH, —O-(lower alkyl), amino which may be substituted with one or two lower alkyl, halogen and R^{Z}.
(h) lower alkyl which may be substituted with one or more groups selected from the substituent groups described in (a) to (g).

The compound of the present invention represented by the general formula (I) may comprise asymmetric carbon atoms depending on the kinds of substituent groups, and optical isomers based on the asymmetric carbon atom may exist. The compound of the present invention includes a mixture of these optical isomers or isolated ones. And, tautomers may exist in the compound of the present invention, and the compound of the present invention includes these isomers as a mixture or an isolated one. And, labeled compound, i.e., compounds wherein one or more atoms are labeled with radioisotopes or non-radioisotopes, are also included in the present invention.

In addition, the compound of the present invention may form a salt, which is included in the present invention as long as pharmaceutically acceptable. Examples of the salt include addition salts with a mineral acid such as hydrochloric acid, hydrobromic acid, hydriodic acid, sulfuric acid, nitric acid, phosphoric acid, and the like; or an organic acid such as formic acid, acetic acid, propionic acid, oxalic acid, malonic acid, succinic acid, fumaric acid, maleic acid, lactic acid, malic acid, tartaric acid, citric acid, methanesulfonic acid, ethanesulfonic acid, benzenesulfonic acid, p-toluenesulfonic acid, aspartic acid, glutamic acid, and the like; or an organic base such as methylamine, ethylamine, ethanolamine, lysine, ornithine, and the like; and ammonium salts, and the like. And a hydrate and a solvate of the compound and its pharmaceutically acceptable salt of the present invention, and those having polymorphism are also included in the present invention. In addition, the compound of the invention also includes a compound which is metabolized in a living body to be converted into the compound of the general formula (I) or its salt, a sôcalled prodrug. As groups forming the prodrug, those described in Prog. Med., 5, pp. 2157-2161, 1985; and Hirokawa-Shoten, 1990, “Development of medicine” Vol. 7, Molecular Design, pp. 163-198 can be exemplified.

### Production Method

The compounds and its pharmaceutically acceptable salt of the present invention can be prepared by various known synthesis methods, using characteristics based on its basic backbone or the kinds of substituent groups. The following describes representative preparation methods. And, according to the kinds of functional groups, it is advantageous in some cases in terms of preparation technique to substitute a functional group with a suitable protection group, i.e., a group that can be easily converted into the functional group, in the starting material or intermediate step. Then, if necessary, the protection group, is removed to obtain a desired compound. Examples of the functional group include hydroxyl, carboxyl, amino group and the like, and examples of the protection group include those described in “Protective Groups in Organic Synthesis”, third edition, edited by Greene and Wuts. It is preferable to suitably use them depending on reaction conditions.

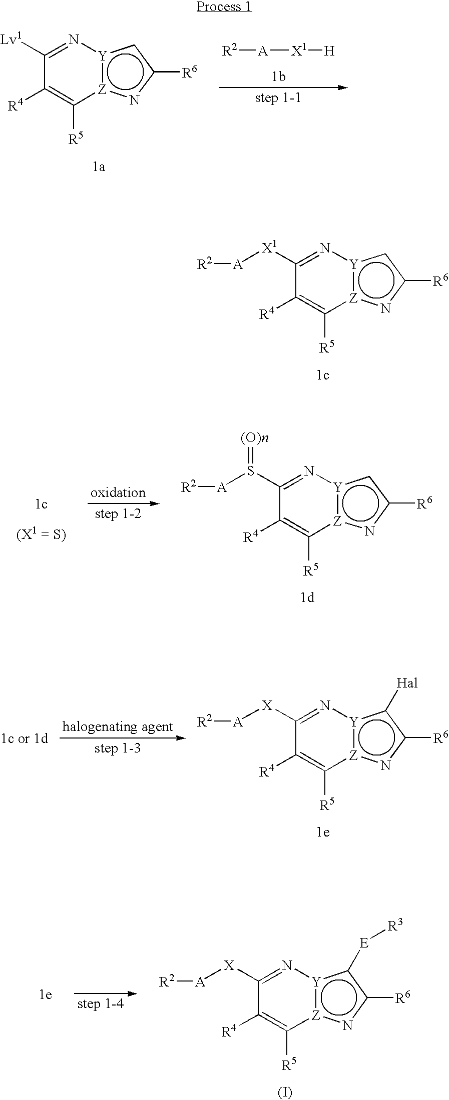

[wherein Lv¹ represents a leaving group, X¹ represents —N(R¹)—, —O— or —S—, n represents 1 or 2, Hal represents halogen, and —R¹, —R², —R³, —R⁴, —R⁵, —R⁶, -A-, -E-, —X—, Y and Z are as defined in the foregoing.]

In this process, the compound (I) is prepared by substitution reaction of 1a with 1b (step 1-1), followed by, if necessary, oxidation step of sulfanyl group into sulfinyl group or sulfonyl group (step 1-2), followed by halogenation of 1c or 1d (step 1-3), and followed by coupling reaction etc. of the thus-prepared compound 1e (step 1-4). Examples of a leaving group include halogen, alkanesulfonyl optionally substituted by one or more halogen, arylsulfonyl and the like.

The compound (I) can be prepared by coupling reaction etc. of 1e. For example, Suzuki coupling, Heck reaction and Sonogashira reaction can be applied, each of which is referred in “Chem. Rev., 95, pp. 2457, 1995”, “J. Am. Soc. Chem., 127, pp. 4685, 2005”, “Synlett, pp. 2329, 2004”, “Tetrahedron Lett., 41, pp. 4363, 2000”, or Tetrahedron Lett., 43, pp. 2695, 2002.”

For the step, substitution reaction can also be applied to prepare the compound (I). The compound 1e can be reacted with a compound “R³-E-H (1f)” in a non-protonic polar solvent such as N,N-dimethylformamide (DMF), N-methyl-2-pyrrolidone, dimethylsulfoxide (DMSO) and the like; an inert organic solvent such as halogenated hydrocarbon including dichloromethane, dichloroethane, chloroform and the like; ether including ether, tetrahydrofuran (THF), dioxane and the like; aromatic hydrocarbon including benzene, toluene, xylene and the like; or water, or a mixture thereof to prepare a compound (I). The reaction is preferably carried out at ambient temperature to reflux temperature of the used solvent.

In order to progress the reaction smoothly, it is advantageous in some cases to employ an excess amount of the compound 1f or carry out the reaction in the presence of a base such as N-methylmorpholine, triethylamine, diethylisopropylamide, N,N-dimethylaniline, pyridine, 4-(N,N-dimethylamino)pyridine, picoline, lutidine and the like.

In another method, when E represents —NHCO—, the compound (I) can be prepared by nitration of 1c or 1d followed by reduction of nitro group into amino group, followed by acylation with “R³—CO₂H (1g).”. A commonly used manner for one skilled in the art can be applied to reduction and acylation. In the acylation step, the reactive derivative such as ester, acid halide, acid anhydride and the like of 1g can also be used. In case 1g is reacted in its free acid form, it is preferable to carry out the reaction using a condensing agent such as dicyclohexylcarbodiimide, carbonyldiimidazole, 1-ethyl-3-(3-dimethylaminopropyl)carbodiimide hydrochloride (EDCI.HCl) and the like. The reaction is, although it varies depending on the reactive derivatives or condensing agent, carried out in an inert solvent such as a halogenated hydrocarbon, aromatic hydrocarbon, ether, DMF, DMSO and the like, under cooling, cooling to ambient temperature, or ambient temperature to heating. In case 1g is reacted in its acid halide form, to progress the reaction smoothly, it is advantageous in some cases to carry out the reaction in the presence of a base.

The compound 1e can be prepared by halogenation of 1c or 1d. As a halogenation agent, those commonly used for a halogen substitution reaction of hydrogen on an aromatic ring can be used. A halogen molecule such as chlorine, bromine, iodine and the like, dioxanedihalide, phenyltrimethylammonium trihalide, a pyridine derivative such as pyridinium hydrohalide perhalide, pyrrolidonehydrotrihalide and the like, a perhalide such as a-pyrrolidone, quaternary ammonium, dioxane and the like are appropriate. An imide-type halogenation agent such as N-iodosuccinimide, N-bromosuccinimide and the like, a hydrogen halide such as hydriodic acid, hydrobromic acid and the like, a metal agent such as copper(II) halide including copper(II) iodide and the like can also be used.

In case of halogen molecule or perhalide is used, the compound 1c or 1d can be reacted in an inert organic solvent such as halogenated hydrocarbon; ether; alcohol including methanol (MeOH), ethanol (EtOH), 2-propanol (iPrOH), ethyleneglycol and the like; aromatic hydrocarbon; acetic acid; ester including ethyl acetate (AcOEt) and the like. If necessary the reaction may be carried out in the presence of a small amount of a catalyst such as hydrogen halide. It is preferable to carry out the reaction at −30° C. to reflux temperature of the used solvent.

In case a hydrogen halide is used as a halogenation agent, the compound 1e can be reacted therewith in an acid solution or a base solution such as sodium hydroxide aqueous solution, and the reaction is preferably carried out at −30° C. to reflux temperature of the used solvent. And in case a metal agent is used as a halogenation agent, the compound 1e is generally dissolved in an inert organic solvent such as halogenated hydrocarbon, ether, alcohol, aromatic hydrocarbon, acetic acid, ester and the like, or water, or a mixture thereof to react with the agent, and if necessary, it is advantageous to carry out the reaction in the presence of a small amount of a catalyst such as hydrogen halide, under ambient temperature to heating.

For oxidation reaction of the step 1-2, oxidation method of sulfur atom of sulfanyl group which is well-known by one skilled in the art is applicable, for example, m-chloroperbenzoic acid, hydrogen peroxide or carboxylic peracid like as acetic peracid or trifluoroacetic peracid can be used for the oxidation.

The step 1-1, which includes a substitution reaction of 1a with 1b (step 1-1), can be carried out in accordance with the step 1-4.

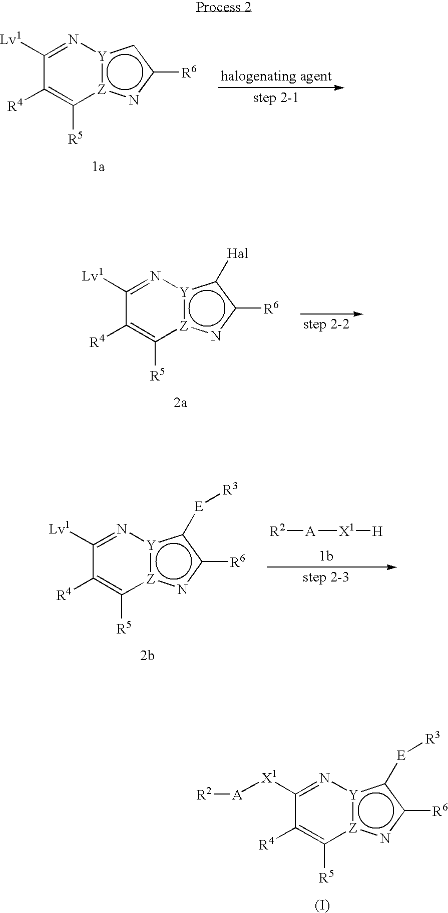

[wherein any symbols are as defined in the foregoing.]

Any steps can be carried out in accordance with the steps of the Process 1.

In addition to the processes as mentioned above, the compound (I) and a salt thereof can be prepared, for example, according to the procedures as illustrated in Examples in the present specification or in a manner similar thereto. The starting compounds can be prepared, for example, according to the procedures as illustrated in Preparations in the present specification or in a manner similar thereto. The compound (I) and a salt thereof can be prepared according to the methods as shown in Preparations or Examples, or in a manner similar thereto.

And, the thus-obtained compounds can be subjected to a process commonly used in the art such as alkylation, acylation, substitution, oxidation, reduction, hydrolysis, and the like to prepare some of the compounds of the general formula (I).

The thus-prepared compound of the present invention is isolated and purified as its free form or as a salt thereof. A salt of the compound (I) can be prepared by subjecting it to a usual salt formation reaction. The isolation and purification are carried out by usual chemical manipulations such as extraction, concentration, evaporation, crystallization, filtration, recrystallization, various types of chromatography and the like.

Various types of isomers can be separated by usual method using the difference in physicochemical properties among isomers. For example, a racemic mixture can be separated by a general racemic mixture resolution method, e.g., a method in which racemic mixture is converted into diastereomer salts with an optically active acid such as tartaric acid and the like and then subjected to optical resolution. And, diastereomers can be separated by fraction crystallization or various types of chromatography or the like. Also, optically active compounds can be prepared using appropriate optically active starting materials.

### INDUSTRIAL APPLICABILITY

In order to show the usefulness of the compound of the present invention, the pharmacological test result of the representative compound of the present invention is shown in the following.

### Assay for Lck Activity

The Src substrate peptide (Upstate) was coated onto 96-well Maxisorp plates (Nunc). Plates were sealed and incubated at 4° C., for 16 hours, washed three times with TBST (20 mM Tris-HCl pH 7.5, 150 mM NaCl and 0.1% Tween-20). The plates were blocked with 0.1% BSA and washed three times with TBST. Purified Lck (Upstate) was incubated in 100 mM Tris-HCl pH 7.5, 125 mM MgCl₂, 25 mM MnCl₂, 2 mM EGTA, 0.25 mM Sodium Vanadate, 2 mM DTT, 0.05 mM ATP with or without inhibitors at 30° C. for 60 minutes and washed with TBST three times. For quantification of phosphorylated tyrosines, plates were incubated with anti-phosphotyrosine, HRP conjugate (4G10, Upstate) for 1 hour at ambient temperature and washed three times with TBST. Detection was carried out using a color reagent, TMB (KPL). Ten minute after TMB addition the OD at 450 nm was measured.

Following IC₅₀ values were obtained: 81 nM for Example 127, 97 nM for Example 153, 460 nM for Example 16, 380 nM for Example 44, 350 nM for Example 47, 310 nM for Example 102, 410 nM for Example 106, and 320 nM for Example 249.

The result clearly suggest that the compound of the present invention have Lck inhibitory activity.

The fused heterocycle and the salt thereof of the present invention have excellent Lck inhibitory activity as shown above. Thus a pharmaceutical composition comprising the compound of the present invention represented by the formula (I) is useful as a therapeutic or prophylactic agent for diseases or conditions caused, by undesirable cytokine signal transduction, such as rejection reaction in organ transplantation, autoimmune diseases, asthma, atopic dermatitis, cancer and leukemia as exemplified below:

Rejection reactions by transplantation of organs or tissues such as the heart, kidney, liver, bone marrow, skin, cornea, lung, pancreas, islet, small intestine, limb, muscle, nerve, intervertebral disc, trachea, myoblast, cartilage, etc.; and graft-versus-host reactions following bone marrow transplantation; autoimmune diseases such as rheumatoid arthritis, systemic lupus erythematosus, Hashimoto's thyroiditis, multiple sclerosis, myasthenia gravis, type I diabetes and complications from diabetes, etc.

Furthermore, a pharmaceutical composition comprising Lck inhibitor such as the compound of the present invention represented by the formula (I) is useful for the therapy or prophylaxis of the following diseases:

Inflammatory or hyperproliferative skin diseases or cutaneous manifestations of immunologically-mediated diseases (e.g., psoriasis, atopic dermatitis, contact dermatitis, eczematoid dermatitis, seborrheic dermatitis, lichen planus, pemphigus, bullous penphigoid, epidermolysis bullosa, urticaria, angioedema, vasculitides, erythema, dermal eosinophilia, lupus erythematosus, acne, alopecia areata, etc.); autoimmune diseases of the eye (e.g., keratoconjunctivitis, vernal conjunctivitis, uveitis associated with Behcet's disease, keratitis, herpetic keratitis, conical keratitis, corneal epithelial dystrophy, keratoleukoma, ocular premphigus, Mooren's ulcer, scleritis, Grave's ophthalmopathy, Vogt-Koyanagi-Harada syndrome, keratoconjunctivitis sicca (dry eye), phlyctenule, iridocyclitis, sarcoidosis, endocrine ophthalmopathy, etc.); reversible obstructive airways diseases [asthma (e.g., bronchial asthma, allergic asthma, intrinsic asthma, extrinsic asthma, dust asthma, etc.), particularly chronic or inveterate asthma (e.g., late asthma, airway hyper-responsiveness, etc.), bronchitis, etc.]; mucosal or vascular inflammations (e.g., gastric ulcer, ischemic or thrombotic vascular injury, ischemic bowel diseases, enteritis, necrotizing enterocolitis, intestinal damages associated with thermal burns, leukotriene B4-mediated diseases, etc.); intestinal inflammations/allergies (e.g., coeliac diseases, proctitis, eosinophilic gastroenteritis, mastocytosis, Crohn's disease, ulcerative colitis, etc.); food-related allergic diseases with symptomatic manifestation remote from the gastrointestinal tract (e.g., migraine, rhinitis, eczema, etc.); autoimmune diseases and inflammatory conditions (e.g., primary mucosal edema, autoimmune atrophic gastritis, premature menopause, male sterility, juvenile diabetes mellitus, pemphigus vulgaris, pemphigoid, sympathetic ophthalmitis, lens-induced uveitis, idiopathic leukopenia, active chronic hepatitis, idiopathic cirrhosis, discoid lupus erythematosus, autoimmune orchitis, arthritis (e.g., arthritis deformans, etc.), polychondritis, etc.); allergic conjunctivitis.

Therefore, the pharmaceutical composition of the present invention is useful for the therapy and prophylaxis of liver diseases [e.g., immunogenic diseases (e.g., chronic autoimmune liver diseases such as autoimmune hepatic diseases, primary biliary cirrhosis, sclerosing cholangitis, etc.), partial liver resection, acute liver necrosis (e.g., necrosis caused by toxins, viral hepatitis, shock, anoxia, etc.), hepatitis B, non-A non-B hepatitis, hepatocirrhosis, hepatic failure (e.g., fulminant hepatitis, late-onset hepatitis, “acute-on-chronic” liver failure (acute liver failure on chronic liver diseases, etc.), etc.), etc.].

The pharmaceutical composition of the present invention can be used in the form of pharmaceutical preparation, for example, in a solid, semisolid or liquid form, which contains the compound of the present invention represented by the formula (I) as an active ingredient in admixture with an organic or inorganic carrier or excipient suitable for external, enteral or parenteral administrations. The active ingredient may be compounded, for example, with the usual non-toxic, pharmaceutically acceptable carriers for tablets, pellets, capsules, suppositories, solutions, emulsions, suspensions, injections, ointments, liniments, eye drops, lotion, gel, cream, and any other form suitable for use.

The carriers those can be used for the present invention include water, glucose, lactose, gum acacia, gelatin, mannitol, starch paste, magnesium trisilicate, talc, cornstarch, keratin, colloidal silica, potato starch, urea and other carriers suitable for use in manufacturing preparations in a solid, semisolid, or liquid form. Furthermore, auxiliary, stabilizing, thickening, solubilizing and coloring agents and perfumes may be used.

For applying the composition to human, it is preferable to apply it by intravenous, intramuscular, topical or oral administration, or by a vascular stent impregnated with the compound (I). In the case of oral administration, a daily dose is approximately 0.0001-50 mg/kg of body weight, preferably approximately 0.001-10 mg/kg, and more preferably approximately 0.01-1 mg/kg, and the daily dose is administered once a day or by dividing it into 2 to 4 doses per day. In the case of intravenous or intramuscular administration, a daily dose is approximately 0.0001-1 mg/kg of body weight, preferably approximately 0.0001-0.1 mg/kg, and the daily dose is administered once a day or by dividing it into plural doses per day. The dose is appropriately decided by taking symptoms, age, and sex of the patient to be treated and the like into consideration.

During the preparation of the above-mentioned pharmaceutical administration forms, the compound (I) or a salt thereof can also be combined together with other immunosuppressive substances, for example rapamycin, mycophenolic acid, cyclosporin A, tacrolimus or brequinar sodium.

The pharmaceutical compositions of the present invention, either from alone or in combination with one of more additional agents which may include but are not limited to cyclosporin A, tacrolimus, sirolimus, everolimus, micophenolate (e.g. Cellcept (R), etc.), azathioprine, brequinar, lefulunomide, fingolimod, anti-IL-2 receptor antibody (e.g. daclizumab, etc.), anti-CD3 antibody (e.g. OKT3, etc.), Anti-T cell immunoglobulin (e.g. AtGam, etc.) aspirin, acetaminophen, ibuprofen, naproxen, piroxicam, and anti inflammatory steroid (e.g. prednisolone or dexamethasone) may be administrated as part of the same or separate dosage forms, via the same or different routes of administration, and on the same or different administration schedules according to standard pharmaceutical practice.

### BEST MODE FOR CARRYING OUT OF THE INVENTION

The following describes the invention more illustratively with reference to Examples, but the present invention is not limited to these examples. In this connection, novel materials are included in the starting materials to be used in the Examples, and production methods of the starting materials from known materials are described as Preparations.

### Preparation 1

The solution of 5-chloropyrazolo[1,5-a]pyrimidine (200 mg) and trans-4-methoxycyclohexanamine (168 mg) in isopropylalcohol (2 ml) was refluxed for 3 hours. After cooling to ambient temperature, the reaction mixture was poured into water, then extracted with ethyl acetate. The organic layer was washed with brine, dried over magnesium sulfate, and evaporated in vacuo. The residue was purified by column chromatography on silica gel eluting with chloroform/methanol (100:0 to 100:10) to give N-(trans-4-methoxycyclohexyl)pyrazolo[1,5-a]pyrimidin-5-amine (70 mg).

¹H-NMR (DMSO-d₆) δ: 1.13-1.34 (4H, m), 1.91-2.08 (4H, m), 3.09-3.20 (1H, m), 3.33 (3H, s), 3.70-3.86 (1H, m), 5.95 (1H, d, J=2.0 Hz), 6.19 (1H, d, J=7.6 Hz), 7.26 (1H, d, J=7.4 Hz), 7.74 (1H, d, J=2.0 Hz), 8.41 (1H, d, J=7.6 Hz).

MS: 247 (M+H)⁺.

### Preparation 2

To a stirred mixture of 6-chloro-3-iodoimidazo[1,2-b]pyridazine (100 mg) and 4-(4,4,5,5-tetramethyl-1,3,2-dioxaborolan-2-yl)pyridine (220 mg) in 1,2-dimethoxyethane (3.3 ml) was added aqueous 2M NaOH aqueous solution (1.08 mL) at ambient temperature. Tetrakis(triphenylphosphine) palladium(0) (24.8 mg) was then added to the mixture at ambient temperature. After addition, the resulting mixture was stirred at 85° C. for 1 hour. The reaction mixture was cooled to ambient temperature and diluted with ethyl acetate/water (20 mL/20 mL). The resulting mixture was acidified with 1M HCl aqueous solution to pH 2 and extracted with ethyl acetate. The aqueous phase was then neutralized by the addition of 2M NaOH aqueous solution to pH 8. The resulting solution was extracted with ethyl acetate three times, the organic layers were combined, dried over magnesium sulfate, and concentrated in vacuo. The residue was purified by silica gel column chromatography eluting with chloroform/methanol (20:1) to give the following compounds.

### 6-Chloro-3-(4-pyridinyl)imidazo[1,2-b]pyridazine

¹H-NMR (CDCl₃) δ: 7.19 (1H, d, J=4.8 Hz), 7.52-7.57 (1H, m), 8.00-8.06 (3H, m), 8.25 (1H, s), 8.73-8.78 (2H, d, J=9.5 Hz).

### 3,6-Di-4-pyridinylimidazo[1,2-b]pyridazine

¹H-NMR (CDCl₃—CD₃OD (9:1)) δ: 7.76 (1H, d, J=9.5 Hz), 7.99 (2H, d, J=6.2 Hz), 8.17 (2H, d, J=6.2 Hz), 8.26 (1H, d, J=9.2 Hz), 8.34 (1H, s), 8.73 (2H, d, J=5.9 Hz), 8.83 (2H, d, J=6.2 Hz).

MS: 274 (M+H)⁺.

### Preparation 3

To a solution of 2-(4-methyl-3-nitrophenoxy)tetrahydro-2H-pyran (4750 mg) in methanol (100 mL) was added 10% palladium on carbon (600 mg). The resulting mixture was stirred under atmospheric hydrogen at ambient temperature for 3 hours. The mixture was filtered through Celite and washed with methanol. The filtrate was concentrated in vacuo to give 2-methyl-5-(tetrahydro-2H-pyran-2-yloxy)aniline (4140 mg).

¹H-NMR (DMSO-d₆) δ: 1.45-1.92 (6H, m), 1.96 (3H, s), 3.45-3.58 (1H, m), 3.68-3.80 (1H, m), 4.79 (2H, bs), 5.25 (1H, t, J=3.0 Hz), 6.12 (1H, dd, J=2.5, 8.5 Hz), 6.29 (1H, d, J=2.5 Hz), 6.76 (1H, d, J=8.5 Hz).

MS: 230 (M+Na)⁺.

The following compound was obtained in a similar manner to that of Preparation 3.

### Preparation 4

### [2-Chloro-5-(tetrahydro-2H-pyran-2-yloxy)phenyl]amine

MS: 269.

### Preparation 5

trans-4-(Imidazo[1,2-b]pyridazin-6-ylamino)cyclohexanol (110 mg) and N-iodosuccinimide (117 mg) in N,N-dimethylformamide (1.1 ml) was stirred at ambient temperature for 4 hours. The reaction mixture was poured into a mixture of 10% sodium thiosulfate aqueous solution and chloroform. Then the organic layer was washed with aqueous saturated sodium hydrogen carbonate, water, brine, dried over magnesium sulfate, and evaporated in vacuo. Resulting precipitates were collected by filtration and washed with diisopropyl ether to give trans-4-[(3-iodoimidazo[1,2-b]pyridazin-6-yl)amino]cyclohexanol as an brown solid (140 mg).

¹H-NMR (DMSO-d₆) δ: 1.12-1.34 (4H, m), 1.77-1.90 (2H, m), 1.94-2.10 (2H, m), 3.38-3.60 (2H, m), 4.57 (1H, d, J=4.0 Hz), 6.59 (1H, d, J=9.9 Hz), 6.73 (1H, d, J=7.3 Hz), 7.34 (1H, brs), 7.64 (1H, d, J=9.9 Hz), 7.79 (1H, brs).

MS: 233 (M+H)⁺.

The following compounds were obtained in a similar manner to that of Preparation 5.

### Preparation 6

### 3-Iodo-N-(trans-4-methoxycyclohexyl)pyrazolo[1,5-a]pyrimidin-5-amine

¹H-NMR (DMSO-d₆) δ: 1.10-1.37 (4H, m), 1.93-2.10 (4H, m), 3.08-3.25 (1H, m), 3.25 (3H, s), 3.72-3.91 (1H, m), 6.23 (1H, d, J=7.5 Hz), 7.5 (1H, d, J=7.3 Hz), 7.81 (1H, s), 8.42 (1H, d, J=7.5 Hz).

MS: 373 (M+H)⁺.

### Preparation 7

### 6-chloro-3-iodo-8-methoxyimidazo[1,2-b]pyridazine

MS: 332 (M+Na)⁺.

### Preparation 8

5-Chloropyrazolo[1,5-a]pyrimidine (100 mg) and N-iodosuccinimide (161 mg) in N,N-dimethylformamide (1 ml) was stirred at ambient temperature for 4 hours. The reaction mixture was poured into a mixture of 10% sodium thiosulfate aqueous solution and chloroform. Then the organic layer was washed with saturated NaHCO₃ aqueous solution, water, brine, dried over magnesium sulfate, and evaporated in vacuo. Resulting precipitates were collected by filtration and washed with diisopropyl ether to give 5-chloro-3-iodopyrazolo[1,5-a]pyrimidine as an brown solid (180 mg).

¹H-NMR (DMSO-d₆) δ: 7.42 (1H, d, J=9.5 Hz), 7.97 (1H, s), 8.23 (1H, d, J=9.2 Hz).

MS: 279 (M+H)⁺.

The following compounds were obtained in a similar manner to that of Preparation 8.

### Preparation 9

### 6-Chloro-3-iodo-7-methylimidazo[1,2-b]pyridazine

MS: 294 (M+H)⁺.

### Preparation 10

### 6-Chloro-3-iodo-8-methylimidazo[1,2-b]pyridazine

MS: 294 (M+H)⁺.

### Preparation 11

To a solution of 6-chloro-3-pyridazinamine (470.8 mg) in N,N-dimethylformamide (5 mL) was added 2-bromo-1,2-diphenylethanone (500 mg) and the mixture was stirred at 80° C. for 5 hours. After all starting material had been consumed, as judged by TLC plate, the reaction mixture was poured into saturated NaHCO₃ aqueous solution (10 mL). The resulting solution was extracted with ethyl acetate three times, the organic layers were combined, dried over magnesium sulfate, and concentrated in vacuo. The residue was purified by silica gel column chromatography eluting with chloroform/methanol (40:1 to 20:1) to give 6-chloro-2,3-diphenylimidazo[1,2-b]pyridazine (286.7 mg).

¹H-NMR (DMSO-d₆) δ: 7.32-7.39 (2H, m), 7.43 (1H, d, J=9.6 Hz), 7.55-7.63 (8H, m), 8.30 (1H, d, J=9.6 Hz).

MS: 306 (M+H)⁺.

### Preparation 12

Trans-4-[(3-iodoimidazo[1,2-b]pyridazin-6-yl)amino]cyclohexanol (300 mg) was dissolved in dimethylsulfoxide (6.0 mL) and methanol (3.6 ml). To this solution were added triethylamine (0.35 mL), palladium acetate (II)(═Pd(OAc)₂, 18.8 mg) and 1,3-bis(diphenylphosphino)propane (=DPPP, 34.5 mg) at ambient temperature. The resulting mixture was stirred at 80° C. for 5 hours under CO (1 atm). After all starting material had been consumed, as judged by TLC plate, the reaction mixture was cooled to ambient temperature and diluted with ethyl acetate/water (10 mL/10 mL). The resulting solution was extracted with ethyl acetate three times, the organic layers were combined, dried over magnesium sulfate, and concentrated in vacuo. The residue was purified by silica gel column chromatography eluting with chloroform/methanol (40:1 to 20:1) to give methyl 6-[(trans-4-hydroxycyclohexyl)amino]imidazo[1,2-b]pyridazine-3-carboxylate (175.1 mg).

¹H-NMR (DMSO-d₆) δ: 1.15-1.40 (4H, m), 1.78-1.97 (2H, m), 2.01-2.20 (2H, m), 3.36-3.68 (—H, m), 3.82 (3H, s), 4.56 (1H, d, J=4.6 Hz), 6.81 (1H, d, J=9.6 Hz), 7.01 (1H, d, J=6.8 Hz), 7.78 (1H, d, J=9.6 Hz), 7.99 (1H, s).

MS: 291 (M+H)⁺.

### Preparation 13

To a solution of Sn (186.9 mg) in conc. HCl aqueous solution (1.9 mL) was added trans-4-[(3-nitroimidazo[1,2-b]pyridazin-6-yl)amino]cyclohexanol (291 mg) at 0° C. for 30 minutes, which was stirred at ambient temperature for 30 minutes. The mixture was diluted with ice-cooled water, basified with ammonium hydroxide, and extracted with n-butylalcohol. The organic layer was washed with brine, dried over sodium sulfate, filtered, and evaporated in vacuo. The residue was purified by column chromatography on silica gel to give trans-4-[(3-aminoimidazo[1,2-b]pyridazin-6-yl)amino]cyclohexanol (282 mg) as a brown amorphous.

MS: 248 (M+H)⁺.

### Preparation 14

To a stirred mixture of 4-methyl-3-nitrophenol (3828 mg) and 3,4-dihydro-2H-pyran (5257 mg) in dichloromethane (1 mL) was added catalytic amount of pyridine 4-methylbenzenesulfonate (628.3 mg) at ambient temperature. The resulting mixture was stirred at ambient temperature for 3 hours. Quenching the reaction with saturated sodium hydrogen carbonate and concentrated in vacuo. The residue was extracted with ethyl acetate. The combined organic layer was washed with brine, dried over magnesium sulfate and concentrated in vacuo. The residue was purified by silica gel column chromatography eluting with n-hexane/ethyl acetate (10:1 to 3:1) to give 2-(4-methyl-3-nitrophenoxy) tetrahydro-2H-pyran (5920 mg).

¹H-NMR (DMSO-d₆) δ: 1.50-1.98 (6H, m), 2.44 (3H, s), 3.51-3.62 (1H, m), 3.63-3.73 (1H, m), 5.58 (1H, t, J=3.0 Hz), 7.30 (1H, dd, J=2.5, 8.5 Hz), 7.42 (1H, d, J=8.5 Hz), 7.61 (1H, d, J=2.5 Hz).

MS: 260 (M+Na)⁺.

The following compound was obtained in a similar manner to that of Preparation 14.

### Preparation 15

### 2-(4-Chloro-3-nitrophenoxy)tetrahydro-2H-pyran

MS: 258 (M+H)⁺.

### Preparation 16

The suspension of benzyl (cis-4-fluorocyclohexyl)carbamate (130 mg) and 10% Pd—C 50% wet (50 mg) in methanol (5 ml) was stirred at ambient temperature for 2 hours under H₂ atmosphere. After filtration, the reaction mixture was evaporated in vacuo to give cis-4-fluorocyclohexanamine (17 mg).

¹H-NMR (DMSO-d₆) δ: 1.23-1.69 (6H, m), 1.79-1.97 (2H, m), 2.64-2.89 (1H, m), 4.57-4.65 (0.5H, m), 4.79-4.89 (0.5H, m).

### Preparation 17

A solution of (diethylamino)sulfur trifluoride (1.48 ml) in dichloromethane (8 ml) was added dropwise to a solution of benzyl (trans-4-hydroxycyclohexyl)carbamate (2.8 g) in dichloromethane (20 ml). After stirring at 0° C. for 1 hour, the reaction mixture was poured into saturated NaHCO₃ aqueous solution, and extracted with dichloromethane. The organic layer was dried over magnesium sulfate, and evaporated in vacuo. The residue was purified by column chromatography on silica gel eluting with n-hexane/ethyl acetate (10:1 to 5:1) to give benzyl (cis-4-fluorocyclohexyl)carbamate (130 mg).

¹H-NMR (CDCl₃) δ: 1.42-2.11 (8H, m), 3.44-3.71 (1H, m), 4.60-4.73 (1H, m), 4.83-4.92 (1H, m), 5.10 (2H, s), 7.30-7.39 (5H, m).

### Preparation 18

6-Chloroimidazo[1,2-b]pyridazine (15 g), trans-4-aminocyclohexanol (11.25 g), sodium tert-butoxide (14.1 g), (R)-2,2′-Bis(diphenylphosphino)-1,1′-binaphthyl (=(R)-BINAP, 1.83 g), and tris(dibenzylideneacetone) dipalladium(0)chloroform adduct (1.01 g) in toluene (430 ml) was refluxed for 1.5 hours. After the reaction mixture was cooled to ambient temperature, dichloromethane (300 ml) and methanol (30 ml) was added and filtrated with Celite pad. The filtrate was evaporated in vacuo. The residue was purified by chromatography on silica gel eluting with chloroform/methanol (100:2 to 10:1) to give trans-4-(imidazo[1,2-b]pyridazin-6-ylamino)cyclohexanol (7.5 g).

¹H-NMR (DMSO-d₆) δ: 1.12-1.34 (4H, m), 1.77-1.90 (2H, m), 1.94-2.10 (2H, m), 3.38-3.60 (2H, m), 4.57 (1H, d, J=4.0 Hz), 6.59 (1H, d, J=9.9 Hz), 6.73 (1H, d, J=7.3 Hz), 7.34 (1H, brs), 7.64 (1H, d, J=9.9 Hz), 7.79 (1H, brs).

MS: 233 (M+H)⁺.

### Preparation 19

A mixture of 6-chloro-3-(4-pyridinyl)imidazo[1,2-b]pyridazine (300 mg) and 28% ammonia aqueous solution (5.0 mL) were heated at 180° C. in a stainless sealed tube for 15 hours. After cooling, the mixture was concentrated under reduced pressure. The resulting crystals were collected by filtration, washed with water and dried under a vacuum to give 3-(4-pyridinyl)imidazo[1,2-b]pyridazin-6-amine (201.3 mg).

¹H-NMR (DMSO-d₆) δ: 6.62 (2H, bs), 6.76 (1H, d, J=9.5 Hz), 7.84 (1H, d, J=9.5 Hz), 8.17 (1H, s), 8.23 (2H, d, J=6.0 Hz), 8.60 (2H, d, J=6.0 Hz).

MS: 212 (M+H)⁺.

### Preparation 20

A mixture of 6-chloro-3-nitroimidazo[1,2-b]pyridazine (193 mg), trans-4-aminocyclohexanol (280 mg) in dimethylsulfoxide (579 μL) was stirred at 70° C. for 7 hours. The resultant was poured into water. The precipitate was filtered, and washed cold water to give trans-4-[(3-nitroimidazo[1,2-b]pyridazin-6-yl)amino]cyclohexanol (244.4 mg) as a yellow powder.

MS: 300 (M+Na)⁺.

### Preparation 21

To a stirred mixture of 6-chloro-3-iodoimidazo[1,2-b]pyridazine (1.00 g) and 4-(4,4,5,5-tetramethyl-1,3,2-dioxaborolan-2-yl)pyridine (1.47 g) in dioxane (30 ml) was added 2M NaOH aqueous solution (5.725 mL) at ambient temperature. The suspension turned to a clear yellow solution. palladium acetate (II)(═Pd(OAc)₂, 40.2 mg) and triphenylphosphine (188 mg) were then added to, the mixture at ambient temperature. After addition, the resulting mixture was stirred at 100° C. for 3 hours. The reaction mixture was cooled to ambient temperature and diluted with ethyl acetate/water (20 mL/20 mL). The resulting mixture was acidified with 1 M HCl aqueous solution to pH 2 and extracted with ethyl acetate. The aqueous phase was then neutralized by the addition of 2 M NaOH aqueous solution to pH 8. The resulting solution was extracted with ethyl acetate three times, the organic layers were combined, dried over magnesium sulfate, and concentrated in vacuo. The residue was purified by silica gel column chromatography eluting with chloroform/methanol (50:1 to 20:1) to give 6-chloro-3-(4-pyridinyl)imidazo[1,2-b]pyridazine.

¹H-NMR (DMSO-d₆) δ: 7.19 (1H, d, J=4.8 Hz), 7.52-7.57 (1H, m), 8.00-8.06 (3H, m), 8.25 (1H, s), 8.73-8.78 (2H, d, J=9.5 Hz).

MS: 231 (M+H)⁺.

The following compounds were obtained in a similar manner to that of Preparation 21.

### Preparation 22

### 6-Chloro-8-Methyl-3-(4-pyridinyl)imidazo[1,2-b]pyridazine

MS: 245 (M+H)⁺.

### Preparation 23

### 6-Chloro-7-methyl-3-(4-pyridinyl)imidazo[1,2-b]pyridazine

MS: 245 (M+H)⁺.

The following compounds were obtained in a similar manner to that of Example 16.

### Preparation 24

### 3-(4-Pyridinyl)imidazo[1,2-b]pyridazin-6-ol

MS: 213 (M+H)⁺.

### Preparation 25

### tert-Butyl(trans-4-{[3-(4-pyridinyl)imidazo[1,2-b]pyridazin-6-yl]amino}cyclohexyl)carbamate

¹H-NMR (DMSO-d₆) δ: 1.21-1.46 (4H, m), 1.40 (9H, s), 1.84-1.96 (2H, m), 2.11-2.21 (2H, m), 3.47-3.64 (2H, m), 6.75 (1H, d, J=9.9 Hz), 6.79 (1H, d, J=7.3 Hz), 7.17 (1H, d, J=6.6 Hz), 7.80 (1H, d, J=9.5 Hz), 8.16 (1H, s), 8.21 (1H, d, J=6.2 Hz), 8.60 (1H, d, J=6.2 Hz).

MS: 409 (M+H)⁺.

### Preparation 26

### tert-Butyl(cis-4-{[3-(4-pyridinyl)imidazo[1,2-b]pyridazin-6-yl]amino}cyclohexyl)carbamate

¹H-NMR (DMSO-d₆) δ: 1.39 (9H, s), 1.59-1.99 (9H, m), 3.72-3.88 (1H, m), 6.78-6.91 (1H, m), 6.86 (1H, d, J=10.0 Hz), 7.05 (1H, d, J=5.5 Hz), 7.8 (1H, d, J=9.5 Hz), 8.16 (1H, s), 8.19 (2H, d, J=6.0 Hz), 8.6 (2H, d, J=6.0 Hz).

MS: 409 (M+H)⁺.

The following compounds were obtained in a similar manner to that of Example 86.

### Preparation 27

### 6-Chloro-3-(2-chlorophenyl)imidazo[1,2-b]pyridazine

¹H-NMR (CDCl₃) δ: 7.12 (1H, d, J=9.5 Hz), 7.38-7.45 (2H, m), 7.55-760 (1H, m), 7.65-7.70 (1H, m), 7.98 (1H, d, J=9.5 Hz), 8.02 (1H, s).

MS: 264 (M+H)⁺.

### Preparation 28

### 6-Chloro-3-(4-phenoxyphenyl)imidazo[1,2-b]pyridazine

¹H-NMR (CDCl₃) δ: 7.03-7.19 (6H, m), 7.38 (2H, t, J=7.3 Hz), 7.92-8.04 (4H, m).

MS: 322 (M+H)⁺.

### Preparation 29

### 6-Chloro-3-[4-(trifluoromethoxy)phenyl]imidazo[1,2-b]pyridazine

¹H-NMR (CDCl₃) δ: 7.14 (1H, d, J=9.5 Hz), 7.38 (2H, d, J=8.8 Hz), 8.00 (1H, d, J=9.5 Hz), 8.08 (1H, s), 8.10 (2H, d, J=8.8 Hz).

MS: 314 (M+H)⁺.

### Preparation 30

### 6-Chloro-3-(4-methoxyphenyl)imidazo[1,2-b]pyridazine

¹H-NMR (DMSO-d₆) δ: 3.83 (3H, s), 7.13 (2H, d, J=9.2 Hz), 7.39 (1H, d, J=9.5 Hz), 8.03 (2H, d, J=8.8 Hz), 8.24 (1H, s), 8.28 (1H, d, J=9.5 Hz).

MS: 260 (M+H)⁺.

### Preparation 31

### 3-(3-Bromophenyl)-6-chloroimidazo[1,2-b]pyridazine

¹H-NMR (DMSO-d₆) δ: 7.48 (1H, d, J=9.5 Hz), 7.53 (1H, d, J=8.1 Hz), 7.60-7.65 (1H, m), 8.12-8.17 (1H, m), 8.33 (1H, d, J=9.5 Hz), 8.34-8.37 (1H, m), 8.43 (1H, s).

MS: 308 (M+H)⁺.

The following compounds were obtained in a similar manner to that of Example 122

### Preparation 32

### 5-Methoxy-2-adamantanamine

MS: 182 (M+H)⁺.

### Preparation 33

### 4-Methoxy-1-adamantanamine

MS: 182 (M+H)⁺.

### Preparation 34

### 4-Fluoro-1-adamantanamine

MS: 170 (M+H)⁺.

### Preparation 35

To a mixture of benzyl 4-amino-1-adamantanol (1.0 g) and benzyl chloridocarbonate (1.02 g) in tetrahydrofuran (10 ml) were added 1M NaOH aqueous solution (5.98 mL) at 0° C. The reaction mixture was stirred for 3 hours at 0° C. After all starting material had been consumed, as judged by TLC plate, the reaction mixture was quenched with saturated KHSO₄ aqueous solution. The resulting solution was extracted with ethyl acetate three times, the organic layers were combined, dried over magnesium sulfate, and concentrated in vacuo. The residue was purified by silica gel column chromatography eluting with chloroform/methanol (98:2 to 90:10) to give benzyl (5-hydroxyadamantan-2-yl)carbamate (1.502 g).

MS: 324 (M+Na)⁺.

The following compound was obtained in a similar manner to that of Preparation 35

### Preparation 36

### Benzyl (4-hydroxyadamantan-1-yl)carbamate

MS: 324 (M+Na)⁺.

### Preparation 37

To a solution of 4-bromo-6-chloro-3-pyridazinamine (500.0 mg) in methanol (10.0 ml), sodium methoxide (518.4 mg) was added at 0° C. The reaction mixture was stirred at 25° C. for 5 hours. After all starting material had been consumed, as judged by TLC plate, the resulting solution was concentrated in vacuo. The residue was poured into water (20 mL). The resulting solution was extracted with ethyl acetate three times, the organic layers were combined, dried over magnesium sulfate, and concentrated in vacuo. The residue was purified by silica gel column chromatography eluting with chloroform/methanol (98:2 to 90:10) to give 6-chloro-4-methoxy-3-pyridazinamine (286.7 mg).

MS: 182 (M+Na)⁺.

### Preparation 38

To a solution of 6-chloro-4-methoxy-3-pyridazinamine (800.0 mg) in water (8.0 ml), chloroacetaldehyde (1.64 ml) was added at ambient temperature. The reaction mixture was stirred at 90° C. for 15 hours. After all starting material had been consumed, as judged by TLC plate, the reaction mixture was cooled to ambient temperature and neutralized with NaHCO₃ aqueous solution. The resulting precipitates were collected by filtration and washed with diisopropyl ether to give 6-chloro-8-methoxyimidazo[1,2-b]pyridazine (702.3 mg).

MS: 184 (M+H)⁺.

### Preparation 39

To a stirred mixture of tert-butyl (trans-4-hydroxycyclohexyl) carbamate (1 g) and triphenylphosphine (7.31 g) in tetrahydrofuran (25 ml) were added dropwise 2,2,2-trifluoroethanol (4.98 mL) and diethylazodicarboxylate (4.39 mL) at 0° C. After stirring at ambient temperature for 96 hours, the reaction mixture was, evaporated in vacuo. The residue was purified by silica gel column chromatography eluting with ethyl acetate/n-hexane (5:1) to give tert-butyl[cis-4-(2,2,2-trifluoroethoxy)cyclohexyl]carbamate (54 mg).

MS: 320 (M+Na)⁺.

The following compounds were obtained in a similar manner to that of Preparation 39.

### Preparation 40

### tert-Butyl(cis-4-phenoxycyclohexyl)carbamate

MS: 314 (M+Na)⁺.

### Preparation 41

### tert-Butyl[cis-4-(phenoxymethyl)cyclohexyl]carbamate

MS: 328 (M+Na)⁺.

The following compound was obtained in a similar manner to that of Example 276.

### Preparation 42

### 4-Bromo-6-chloro-3-pyridazinamine

MS: 232 (M+Na)⁺.

### Preparation 43

To a stirred mixture of benzyl (5-hydroxyadamantan-2-yl) carbamate (2.0 g) and trimethyloxonium tetrafluoroborate (1.963 g) in dichloromethane (20 ml) were added 2,6-di-tert-butyl-4-methylpyridine (3.407 g) at ambient temperature. The reaction mixture was refluxed for 3 hours. After all starting material had been consumed, as judged by TLC plate, the reaction mixture was cooled to ambient temperature. After cooling, the solvent and reagent were evaporated. Resultings were triturated by ethyl acetate to remove white powder. The filtration was diluted with ethyl acetate/water (50 mL/50 mL). The resulting solution was extracted with ethyl acetate three times, the organic layers were combined, dried over magnesium sulfate, and concentrated in vacuo. The residue was purified by silica gel column chromatography eluting with n-hexane/ethyl acetate (95:5 to 70:30) to give benzyl (5-methoxyadamantan-2-yl) carbamate (1.04 g).

MS: 338 (M+Na)⁺.

The following compound was obtained in a similar manner to that of Preparation 43.

### Preparation 44

### Benzyl (4-methoxyadamantan-1-yl)carbamate

MS: 338 (M+Na)⁺.

### Preparation 45

To a suspension of 60% NaH (122 mg) in N,N-dimethylformamide (3 mL) was added 2-(trans-4-hydroxycyclohexyl)-1H-isoindole-1,3(2H)-dione (500 mg) at 0° C. After stirring at ambient temperature for 0.5 hour, bromoethyl methylether was added to this reaction mixture at 0° C. The reaction mixture was stirred at ambient temperature for 3 hours. The reaction mixture was poured into saturated NH₄Cl aq. and extracted with ethyl acetate three times. The combined extracts were washed with water and brine, dried over magnesium sulfate and concentrated under reduced pressure. The residue was purified by silica gel column chromatography eluting with ethyl acetate/n-hexane (1:1) to give 2-[trans-4-(2-methoxyethoxy)cyclohexyl]-1H-isoindole-1,3(2H)-dione (130 mg).

MS: 326 (M+Na)⁺.

The following compounds were obtained in a similar manner to that of Preparation 45.

### Preparation 46

### tert-Butyl{[trans-4-(1,3-dioxo-1,3-dihydro-2H-isoindol-2-yl)cyclohexyl]oxy}acetate

MS: 382 (M+Na)⁺.

The following compounds were obtained in a similar manner to that of Example 140.

### Preparation 47

### Benzyl (4-fluoroadamantan-1-yl)carbamate

MS: 326 (M+Na)⁺.

### Preparation 48

### tert-Butyl (trans-4-fluorocyclohexyl)carbamate

MS: 240 (M+Na)⁺.

### Preparation 49

To the solution of {[trans-4-(1,3-dioxo-1,3-dihydro-2H-isoindol-2-yl)cyclohexyl]oxy}acetic acid (303 mg) in dichloromethane (3 mL) was added oxallylchloride (0.174 mL) at ambient temperature. To the solution was added a few portion of N,N-dimethylformamide. After stirring at ambient temperature for 45 minutes. Then, the solution was concentrated. Resulting residue was dissolved to tetrahydrofuran (5 ml), and the solution was added dropwise to the solution of N-methylmethanamine (2M in methanol, 1 mL) in tetrahydrofuran (2 ml) at 0° C. The mixture was stirred for 2 hours at this temperature. To the mixture was added water and was extracted with ethyl acetate. The combined organics were dried over magnesium sulfate, and concentrated to give 2-{[trans-4-(1,3-dioxo-1,3-dihydro-2H-isoindol-2-yl)cyclohexyl]oxy}-N,N-dimethylacetamide.

MS: 353 (M+Na)⁺.

### Preparation 50

A mixture of 6-chloro-3-nitroimidazo[1,2-b]pyridazine (6 g), [(1S)-1-phenylethyl]amine (11.6 g) in dimethylsulfoxide (18 mL) was stirred at 80° C. for 14 hours. The reaction mixture was cooled to ambient temperature and diluted with water. The resulting solution was extracted with ethyl acetate three times. The combined extracts were washed with water and brine, dried over magnesium sulfate and concentrated under reduced pressure. The residue was purified by silica gel column chromatography eluting with ethyl acetate/n-hexane (2:1) to give 3-nitro-N-[(1S)-1-phenylethyl]imidazo[1,2-b]pyridazin-6-amine (7.6 g).

MS: 306 (M+Na)⁺.

### Preparation 51

To a stirred solution of 2-[trans-4-(2-methoxyethoxy)cyclohexyl]-1H-isoindole-1,3(2H)-dione (110 mg) in tetrahydrofuran/ethanol (2 mL/2 mL) was added hydrazine hydrate (0.07 mL) at ambient temperature. After stirring for 4.5 hours under reflux, the reaction mixture was poured into 1M NaOH, aqueous solution and extracted with chloroform/methanol (9:1). The organic layer was dried over magnesium sulfate, and concentrated under reduced pressure. 4M HCl in 1,4-dioxane (453 μl) was added to the solution of this residue in methanol (5 ml) under stirring at 0° C. After stirring at ambient temperature for 1 hour, the reaction mixture was evaporated in vacuo to give trans-4-(2-methoxyethoxy)cyclohexanamine hydrochloride.

MS: 174 (Mfree+H)⁺.

### Preparation 52

To a stirred solution of 2-{[trans-4-(1,3-dioxo-1,3-dihydro-2H-isoindol-2-yl)cyclohexyl]oxy}-N,N-dimethylacetamide (180 mg) in tetrahydrofuran/ethanol (3 mL/3 mL) was added hydrazine hydrate (0.106 mL) at ambient temperature. After stirring for 4.5 hours under reflux, the reaction mixture was poured into 1M NaOH aqueous solution and extracted with chloroform/methanol (8:1). The organic layer was dried over magnesium sulfate, and concentrated under reduced pressure to give 2-[(trans-4-aminocyclohexyl)oxy]-N,N-dimethylacetamide.

MS: 201 (M+H)⁺.

### Preparation 53

To a stirred solution of 3-nitro-N-[(1S)-1-phenylethyl]imidazo[1,2-b]pyridazin-6-amine (567 mg) in ethanol (30 ml) were added FeCl₃ (32.4 mg), activated carbon (280 mg) and hydrazine hydrate (0.388 mL) at 80° C. The reaction mixture was stirred at this temperature for 2 hours. After filtration, the reaction mixture was evaporated in vacuo. The residue was purified by silica gel column chromatography eluting with chloroform/methanol (9:1) to give N-6-[(1S)-1-phenylethyl]imidazo[1,2-b]pyridazine-3,6-diamine (450 mg).

MS: 254 (M+H)⁺.

The following compounds were obtained in a similar manner to that of Example 223.

### Preparation 54

### cis-4-Phenoxycyclohexanamine hydrochloride

MS: 192 (Mfree+H)⁺.

### Preparation 55

### cis-4-(Phenoxymethyl)cyclohexanamine hydrochloride

MS: 206 (Mfree+H)⁺.

### Preparation 56

### cis-4-(2,2,2-Trifluoroethoxy)cyclohexanamine hydrochloride

MS: 198 (Mfree+H)⁺.

### Preparation 57

### trans-4-Fluorocyclohexanamine hydrochloride

MS: 118 (Mfree+H)⁺.

### Preparation 58

### {[trans-4-(1,3-Dioxo-1,3-dihydro-2H-isoindol-2-yl)cyclohexyl]oxy}acetic acid

MS: 326 (M+Na)⁺.

The following compound was obtained in a similar manner to that of Preparation 21.

### Example 1

### N-(trans-4-Methoxycyclohexyl)-3-(4-pyridinyl)pyrazolo[1,5-a]pyrimidin-5-amine

¹H-NMR (DMSO-d₆) δ: 1.25-1.40 (4H, m), 1.75-2.00 (4H, m), 3.00-3.17 (1H, m), 3.22 (3H, s), 3.79-3.96 (1H, m), 6.31 (1H, d, J=7.6 Hz), 7.40-7.50 (1H, m), 7.70-7.85 (3H, m), 8.40-8.57 (3H, m).

MS: 324 (M+H)⁺.

### Example 2

To a solution of trans-4-[(3-aminoimidazo[1,2-b]pyridazin-6-yl)amino]cyclohexanol (30 mg) in pyridine (1.0 mL) was added N,O-bis(trimethylsilyl)acetamide (30 μL), which was stirred at ambient temperature for 10 minutes. To the mixture was added isonicotinoyl chloride hydrochloride (23.8 mg) at 0° C., which was stirred at ambient temperature for 24 hours. To the resultant was added water. The mixture was extracted with dichloromethane. The organic layer was washed with brine, dried over sodium sulfate, filtered, and evaporated in vacuo. The residue was purified by preparative TLC to give N-{6-[(trans-4-hydroxycyclohexyl)amino]imidazo[1,2-b]pyridazin-3-yl}isonicotinamide (14 mg) as a yellow powder.

MS: 353 (M+H)⁺.

### Example 3

To a solution of N-[2-methyl-5-(tetrahydro-2H-pyran-2-yloxy)phenyl]-3-(4-pyridinyl)imidazo[1,2-b]pyridazin-6-amine (70 mg) in methanol (1 mL) was added catalytic amount of pyridinium p-toluenesulfonate (=PPTS, 8.76 mg) at ambient temperature. The resulting mixture was stirred at 50° C. for 1 hour. Quenching the reaction with saturated sodium hydrogen carbonate and concentrated in vacuo. The residue was extracted with ethyl acetate. The combined organic layers were washed with brine, dried over magnesium sulfate, and concentrated in vacuo. The residue was purified by silica gel column chromatography eluting with chloroform/methanol (50:1 to 20:1) to give 4-methyl-3-{[3-(4-pyridinyl)imidazo[1,2-b]pyridazin-6-yl]amino}phenol (27.6 mg).

¹H-NMR (DMSO-d₆) δ: 2.16 (3H, s), 6.57 (1H, dd, J=2.5, 8.0 Hz), 7.08 (1H, dd, J=2.5, 8.0 Hz), 7.09 (1H, d, J=10.0 Hz), 7.17 (1H, d, J=2.0 Hz), 7.97 (1H, d, J=10.0 Hz), 8.11 (2H, d, J=6.0 Hz), 8.25 (1H, s), 8.50 (2H, d, J=6.0 Hz), 8.63 (1H, bs), 9.28 (1H, bs).

MS: 318 (M+H)⁺.

The following compounds were obtained in a similar manner to that of Example 3.

### Example 4

### 4-Chloro-3-{[3-(4-pyridinyl)imidazo[1,2-b]pyridazin-6-yl]amino}phenol

MS: 338 (M+H)⁺.

### Example 5

### 4-Methyl-3-{[8-methyl-3-(4-pyridinyl)imidazo[1,2-b]pyridazin-6-yl]amino}phenol

¹H-NMR (DMSO-d₆) δ: 2.08 (3H, s), 2.42 (3H, s), 6.67 (1H, dd), 6.87 (1H, d), 7.14 (1H, d), 7.85 (1H, s), 7.92 (2H, d), 8.02 (1H, s), 8.18 (1H, s), 8.36 (2H, d), 9.27 (1H, s).

MS: 332 (M+Na)⁺.

### Example 6

CF₃CO₂H (35 μL) was added to a solution of tert-butyl trans-4-{[3-(4-pyridinyl)imidazo[1,2-b]pyridazin-6-yl]amino}cyclohexanecarboxylate (9 mg) in dichloromethane (1 ml). After stirring at ambient temperature for overnight, the reaction mixture was evaporated in vacuo to give trans-4-{[3-(4-pyridinyl)imidazo[1,2-b]pyridazin-6-yl]amino}cyclohexanecarboxylic acid bis(trifluoroacetate) (9 mg).

¹H-NMR (DMSO-d₆): 1.17-2.35 (8H, m), 3.52-3.75 (2H, m), 6.96 (1H, d, J=9.8 Hz), 7.47 (1H, d, J=6.1 Hz), 7.93 (1H, d, J=9.8 Hz) 8.60 (1H, s), 8.70 (2H, d, J=6.3 Hz), 8.87 (2H, d, J=6.3 Hz).

MS: 338 (Mfree+H)⁺.

### Example 7

A mixture of 6-chloro-3-(4-pyridinyl)imidazo[1,2-b]pyridazine (80 mg) and (3-pyridinylmethyl)amine (375.1 mg) were subjected to microwave irradiation at 180° C. for 1 hour. After all starting material had been consumed, as judged by TLC plate, the reaction mixture was cooled to ambient temperature and diluted with ethyl acetate/water (10 mL:10 mL). The resulting solution was extracted with ethyl acetate three times, the organic layers were combined, dried over magnesium sulfate, and concentrated in vacuo. The residue was purified by silica gel column chromatography eluting with chloroform/methanol (40:1 to 20:1) to give 3-(4-pyridinyl)-N-(3-pyridinylmethyl)imidazo[1,2-b]pyridazin-6-amine (56.3 mg).

¹H-NMR (DMSO-d₆) δ: 4.57 (2H, d, J=5.4 Hz), 6.88 (1H, d, J=9.7 Hz), 7.39 (1H, dd, J=4.8, 7.7 Hz), 7.80-7.94 (2H, m), 7.86 (1H, d, J=9.7 Hz), 7.95 (2H, d, J=6.3 Hz), 8.14 (1H, s), 8.47 (1H, d, J=3.5 Hz), 8.53 (2H, d, J=6.3 Hz), 8.67 (1H, s).

MS: 303 (M+H)⁺.

The following compounds were obtained in a similar manner to that of Example 7.

### Example 8

(±)-N-[3-(4-Pyridinyl)imidazo[1,2-b]pyridazin-6-yl]-1,2-cyclohexanediamine.

MS: 309 (M+H)⁺.

### Example 9

### N-(2-Fluorobenzyl)-3-(4-pyridinyl)imidazo[1,2-b]pyridazin-6-amine

¹H-NMR (DMSO-d₆) δ: 4.58 (2H, d, J=5.6 Hz), 6.89 (1H, d, J=9.6 Hz), 7.14-7.18 (1H, m), 7.27-7.34 (2H, m), 7.44-7.49 (1H, m), 7.84-7.87 (2H, m), 7.96-7.98 (2H, m), 8.13 (1H, s), 8.50-8.52 (2H, m).

MS: 320 (M+H)⁺.

### Example 10

### 3-({[3-(4-Pyridinyl)imidazo[1,2-b]pyridazin-6-yl]amino}methyl)phenol

¹H-NMR (DMSO-d₆) δ: 4.44 (2H, d, J=5.6 Hz), 6.63-6.66 (1H, m), 6.83-6.89 (3H, m), 7.16 (1H, t, J=7.8 Hz), 7.78-7.82 (1H, m), 7.83 (1H, d, J=9.6 Hz), 7.98-8.00 (2H, m), 8.13 (1H, s), 8.52-8.54 (2H, m), 9.33 (1H, brs)

MS: 318 (M+H)⁺.

### Example 11

### 2-({[3-(4-Pyridinyl)imidazo[1,2-b]pyridazin-6-yl]amino}methyl)phenol

¹H-NMR (DMSO-d₆) δ: 4.47 (2H, d, J=5.4 Hz), 6.72-6.77 (1H, m), 6.89 (1H, d, J=9.6 Hz), 6.90-6.92 (1H, m), 7.05-7.09 (1H, m), 7.24-7.26 (1H, m), 7.60-6.63 (1H, m), 7.82 (1H, d, J=9.6 Hz), 8.04-8.06 (2H, m), 8.13 (1H, s), 8.52-8.54 (2H, m), 9.62 (1H, brs).

MS: 318 (M+H)⁺.

### Example 12

### (1S,2R,4R)-4-{[3-(4-Pyridinyl)imidazo[1,2-b]pyridazin-6-yl]amino}-1,2-cyclohexanediol

¹H-NMR (DMSO-d₆) δ: 1.40-2.08 (6H, m), 3.55-3.81 (3H, m), 4.29 (1H, d, J=2.6 Hz), 4.59 (1H, d, J=5.5 Hz), 6.75 (1H, d, J=9.8 Hz), 7.14 (1H, d, J=7.0 Hz), 7.79 (1H, d, J=9.8 Hz), 8.16 (1H, s), 8.21 (2H, a, J=6.3 Hz), 8.60 (2H, d, J=6.3 Hz).

MS: 326 (M+H)⁺.

### Example 13

### (1R,2S,4R)-4-{[3-(4-Pyridinyl)imidazo[1,2-b]pyridazin-6-yl]amino}-1,2-cyclohexanediol

¹H-NMR (DMSO-d₆) δ: 1.25-1.46 (2H, m), 1.56-1.85 (2H, m), 1.91-2.12 (1H, m), 2.16-2.36 (1H, m), 3.48-3.62 (1H, m), 3.80-3.92 (1H, m), 3.95-4.18 (1H, m), 4.48 (1H, d, J=2.6 Hz), 4.49 (1H, d, J=5.5 Hz), 6.75 (1H, d, J=9.7 Hz), 7.03 (1H, d, J=6.8 Hz), 7.78 (1H, d, J=9.7 Hz), 8.18 (1H, s), 8.29 (2H, d, J=6.2 Hz), 8.59 (2H, d, J=6.2 Hz).

MS: 326 (M+H)⁺.

### Example 14

### (2S)-2-Phenyl-2-{[3-(4-pyridinyl)imidazo[1,2-b]pyridazin-6-yl]amino}ethanol

MS: 332 (M+H)⁺.

### Example 15

### (2R)-2-Phenyl-2-{[3-(4-pyridinyl)imidazo[1,2-b]pyridazin-6-yl]amino}ethanol

¹H-NMR (DMSO-d₆) δ: 3.60-3.80 (2H, m), 4.82 (1H, dd, J=7.3, 13.4 Hz), 5.07 (1H, dd, J=5.7, 5.7 Hz), 7.01 (1H, d, J=9.6 Hz), 7.18-7.51 (5H, m), 7.77 (1H, d, J=6.0 Hz), 7.82 (1H, d, J=9.6 Hz), 7.85 (2H, d, J=6.1 Hz), 8.09 (1H, s), 8.52 (2H, d, J=6.1 Hz).

MS: 332 (M+H)⁺.

### Example 16

To a solution of 6-chloro-3-(4-pyridinyl)imidazo[1,2-b]pyridazine (653 mg) and tetrahydro-2H-pyran-4-amine (859.1 mg) in toluene (56.8 mL) was added tris(dibenzylidenacetone) dipalladium chloroform complex(═Pd₂ dba₃.CHCl₃, 87.9 mg), 2,2′-Bis(diphenylphosphino)-1,1′-binaphthyl (=BINAP, 158.7 mg) and sodium tert-butoxide (1.22 g). The mixture was stirred at 110° C. for 1.5 hours under nitrogen atmosphere. The resultant was poured into a mixture of water and dichloromethane, and acidified with 1 M HCl aqueous solution (pH 3). The aqueous phase was separated, adjusted to pH 8.5 by 1M NaOH aqueous solution, and extracted with dichloromethane. The organic phase was separated, washed with brine, and dried over sodium sulfate. Evaporation of the solvent gave a residue, which was purified by column chromatography on silica gel to give 3-(4-pyridinyl)-N-(tetrahydro-2H-pyran-4-yl)imidazo[1,2-b]pyridazin-6-amine (710 mg) as a yellow powder.

¹H-NMR (DMSO-d₆) δ: 1.52 (2H, ddd, J=23.6, 11.0, 4.0 Hz), 2.01 (2H, d, J=10.5 Hz), 3.52 (2H, dt, J=2.0, 11.3 Hz), 3.81-4.02 (3H, m), 6.78 (1H, d, J=9.7 Hz), 7.24 (1H, d, J=6.6 Hz), 7.83 (1H, d, J=9.7 Hz), 8.17 (1H, s), 8.19 (2H, d, J=6.2 Hz).

The following compounds were obtained in a similar manner to that of Example 16.

### Example 17

### 3-(4-Pyridinyl)-N-(tetrahydro-2-furanylmethyl)imidazo[1,2-b]pyridazin-6-amine

¹H-NMR (CDCl₃-CD₃OD (9:1)) δ: 1.63-1.81 (1H, m), 1.91-2.18 (3H, m), 3.32-3.44 (1H, m), 3.61-3.71 (1H, m), 3.79-3.99 (2H, m), 4.18-4.32 (1H, m), 5.71-5.80 (1H, m), 6.69 (1H, d, J=9.5 Hz), 7.68 (1H, d, J=9.5 Hz), 7.94 (1H, s), 8.11 (2H, d, J=6.6 Hz), 8.59 (2H, d, J=6.2 Hz).

MS: 296 (M+H)⁺.

### Example 18

### N-(cis-4-Methoxycyclohexyl)-3-(4-pyridinyl)imidazo[1,2-b]pyridazin-6-amine

¹H-NMR (DMSO-d₆) δ: 1.53-1.92 (8H, m), 3.25 (3H, s), 3.34-3.42 (1H, m), 3.70-3.83 (1H, m), 6.79 (1H, d, J=9.7 Hz), 7.12 (1H, d, J=6.7 Hz), 7.8 (1H, d, J=9.7 Hz), 8.16 (1H, s), 8.2 (2H, dd, J=1.6 Hz, 4.6 Hz), 8.6 (2H, dd, J=1.6 Hz, 4.6 Hz).

MS: 324 (M+H)⁺.

### Example 19

### 3-{[3-(4-Pyridinyl)imidazo[1,2-b]pyridazin-6-yl]amino}-1-adamantanol

¹H-NMR (DMSO-d₆) δ: 1.42-1.74 (6H, m), 1.91-2.34 (6H, m), 2.17-2.34 (2H, m), 4.62 (1H, s), 6.79 (1H, d, J=9.8 Hz), 6.87 (1H, s), 7.77 (1H, d, J=9.8 Hz), 8.13 (1H, s), 8.18 (2H, d, J=6.1 Hz), 8.60 (2H, d, J=6.1 Hz).

MS: 362 (M+H)⁺.

### Example 20

### N-(4,4-Difluorocyclohexyl)-3-(4-pyridinyl)imidazo[1,2-b]pyridazin-6-amine

MS: 330 (M+H)⁺.

### Example 21

### trans-4-{[3-(2-Chlorophenyl)imidazo[1,2-b]pyridazin-6-yl]amino}cyclohexanol

¹H-NMR (CDCl₃) δ: 1.16-1.45 (4H, m), 1.96-2.07 (2H, m), 2.14-2.24 (2H, m), 3.51-3.78 (2H, m), 4.12 (1H, d, J=6.6 Hz), 6.42 (1H, d, J=9.5 Hz), 7.31-7.39 (2H, m), 7.51-7.55 (1H, m), 7.69 (1H, d, J=9.5 Hz), 7.76-7.83 (1H, m), 7.80 (1H, s).

MS: 343 (M+H)⁺.

### Example 22

### (1S,2R)-2-{[3-(4-Pyridinyl)imidazo[1,2-b]pyridazin-6-yl]amino}cyclohexanol

¹H-NMR (DMSO-d₆) δ: 1.29-1.48 (2H, m), 1.52-1.84 (6H, m), 3.72-3.86 (1H, m), 4.03-4.11 (1H, m), 4.70 (1H, d, J=4.0 Hz), 6.94 (1H, d, J=9.9 Hz), 6.95 (1H, d, J=7.0 Hz), 7.78 (1H, d, J=9.5 Hz), 8.19 (2H, d, J=6.6 Hz), 8.60 (2H, d, J=6.2 Hz).

MS: 310 (M+H)⁺.

### Example 23

### 3-(3-Bromophenyl)-N-(2-thionylmethyl)imidazo[1,2-b]pyridazin-6-amine

¹H-NMR (CDCl₃) δ: 4.80 (2H, s), 6.5 (1H, d, J=9.5 Hz), 6.99 (1H, dd, J=1.8, 3.3 Hz), 7.09-7.12 (1H, m), 7.24 (1H, dd, J=1.1, 5.1 Hz), 7.31 (1H, t, J=7.7 Hz), 7.42-7.47 (1H, m), 7.72 (1H, d, J=9.5 Hz), 7.84 (1H, brs), 7.94-8.00 (1H, m), 8.35-8.38 (1H, m).

MS: 385 (M+H)⁺.

### Example 24

### N,N-Dimethyl-3-(4-pyridinyl)imidazo[1,2-b]pyridazin-6-amine

¹H-NMR (CDCl₃-CD₃OD (95:5)) δ: 3.20 (6H, s), 6.87 (1H, d, J=9.9 Hz), 7.78 (1H, d, J=9.9 Hz), 8.02 (1H, s), 8.10 (2H, d, J=5.9 Hz), 8.66 (2H, brs).

MS: 240 (M+H)⁺.

### Example 25

### N-(2-Methoxyethyl)-3-(4-pyridinyl)imidazo[1,2-b]pyridazin-6-amine

¹H-NMR (CDCl₃-CD₃OD (9:1)) δ: 3.44 (3H, s), 3.60-3.66 (2H, m), 3.68-3.74 (2H, m), 6.68 (1H, d, J=9.9 Hz), 7.68 (1H, d, J=9.9 Hz), 7.95 (1H, s), 8.11 (2H, d, J=6.2 Hz), 8.59 (2H, d, J=6.2 Hz).

MS: 270 (M+H)⁺.

### Example 26

### 3-(4-Pyridinyl)-N-(2-thienylmethyl)imidazo[1,2-b]pyridazin-6-amine

¹H-NMR (CDCl₃-CD₃OD (9:1)) δ: 4.79 (2H, d, J=0.73 Hz), 6.69 (1H, d, J=9.5 Hz), 6.98-7.03 (1H, m), 7.08-7.11 (1H, m), 7.23-7.27 (1H, m), 7.70 (1H, d, J=9.9 Hz), 7.94 (1H, s), 8.04 (2H, d, J=6.2 Hz), 8.56 (2H, d, J=6.2H z).

MS: 308 (M+H)⁺.

### Example 27

(cis-4-{[3-(4-Pyridinyl)imidazo[1,2-b]pyridazin-6-yl]amino}cyclohexyl)methanol.

¹H-NMR (DMSO-d₆) δ: 1.00-1.48 (5H, m), 1.78-1.90 (2H, m), 2.14-2.26 (2H, m), 3.28 (2H, d, J=6.0 Hz), 3.51-3.69 (1H, m), 4.45 (1H, t, J=6.0 Hz), 6.75 (1H, d, J=9.5 Hz), 7.13 (1H, d, J=6.5 Hz), 7.79 (1H, d, J=9.5 Hz), 8.16 (1H, s), 8.21 (2H, d, J=6.0 Hz), 8.6 (2H, d, J=6.0 Hz).

MS: 324 (M+H)⁺.

### Example 28

### N-[(1R,2R)-2-(Benzyloxy)cyclohexyl]-3-(4-pyridinyl)imidazo[1,2-b]pyridazin-6-amine

¹H-NMR (DMSO-d₆) δ: 1.21-1.58 (4H, m), 1.62-1.86 (2H, m), 1.97-2.26 (2H, m), 3.38-3.64 (1H, m), 3.75-3.98 (1H, m), 4.52 (1H, d, J=11.5 Hz), 4.62 (1H, d, J=11.5 Hz), 6.86 (1H, d, J=9.8 Hz), 7.10-7.29 (6H, m), 7.80 (1H, d, J=9.5 Hz), 8.14 (1H, s), 8.19 (2H, dd, J=1.5, 5.0 Hz), 8.56 (2H, dd, J=1.5, 4.5 Hz).

MS: 400 (M+H)⁺, 422 (M+Na)⁺.

### Example 29

### N-(3-Methoxy-1-adamantyl)-3-(4-pyridinyl)imidazo[1,2-b]pyridazin-6-amine

¹H-NMR (DMSO-d₆) δ: 1.56-1.75 (6H, m), 1.97-2.18 (6H, m), 2.26-2.35 (2H, m), 3.13 (3H, s), 6.8 (1H, d, J=9.8 Hz), 6.93 (1H, s), 7.78 (1H, d, J=9.8 Hz), 8.13 (1H, s), 8.17 (2H, d, J=6.2 Hz), 8.6 (2H, d, J=6.2 Hz).

MS: 376 (M+H)⁺.

### Example 30

### N-(cis-4-Fluorocyclohexyl)-3-(4-pyridinyl)imidazo[1,2-b]pyridazin-6-amine

¹H-NMR (DMSO-d₆) δ: 1.49-2.09 (8H, m), 3.64-3.86 (1H, m), 4.73 (0.5H, br), 4.97 (0.5H, br), 6.55 (0.5H, s), 6.79 (1H, d, J=10.0 Hz), 7.19 (1H, d, J=7.0 Hz), 7.81 (1H, d, J=9.5 Hz), 8.17 (0.5H, s), 8.2 (2H, d, J=6.0 Hz), 8.6 (2H, d, J=6.0 Hz).

MS: 312 (M+H)⁺.

### Example 31

### N-[trans-4-(Methoxymethyl)cyclohexyl]-3-(4-pyridinyl) imidazo[1,2-b]pyridazin-6-amine

¹H-NMR (DMSO-d₆) δ: 1.02-1.35 (4H, m), 1.53-1.66 (1H, m), 1.77-1.91 (2H, m), 2.14-2.27 (2H, m), 3.21 (2H, d, J=6.3 Hz), 3.26 (3H, s), 3.51-3.67 (1H, m), 6.75 (1H, d, J=9.5 Hz), 7.13 (1H, d, J=7.0 Hz), 7.79 (1H, d, J=9.5 Hz), 8.16 (1H, s), 8.2 (2H, dd, J=1.5 Hz, 4.5 Hz), 8.6 (2H, dd, J=1.5H z, 4.5 Hz).

MS: 338 (M+H)⁺.

### Example 32

### trans-4-{[3-(4-Phenoxyphenyl)imidazo[1,2-b]pyridazin-6-yl]amino}cyclohexanol

¹H-NMR (CDCl₃) δ: 1.21-1.67 (4H, m), 2.00-2.12 (2H, m), 2.22-2.34 (2H, m), 3.63-3.81 (2H, m), 4.19 (1H, d, J=7.3 Hz), 6.40 (1H, d, J=9.5 Hz), 7.05-7.17 (3H, m), 7.11 (2H, d, J=8.8 Hz), 7.33-7.41 (2H, m), 7.68 (1H, d, J=9.9 Hz), 7.77 (1H, s), 8.07 (2H, d, J=9.2 Hz).

MS: 401.2 (M+H)⁺.

### Example 33

### trans-4-({3-[4-(Trifluoromethoxy)phenyl]imidazo[1,2-b]pyridazin-6-yl}amino)cyclohexanol

¹H-NMR (CDCl₃-CD₃OD (9:1)) δ: 1.22-1.55 (4H, m), 1.99-2.13 (2H, m), 2.20-2.32 (2H, m), 3.59-3.77 (2H, m), 6.54 (1H, d, J=9.2 Hz), 7.30 (2H, d, J=9.5 Hz), 7.62 (1H, d, J=9.5 Hz), 7.73 (1H, s), 8.15 (2H, d, J=8.8 Hz).

MS: 393 (M+H)⁺.

### Example 34

### trans-4-{[3-(4-Methoxyphenyl)imidazo[1,2-b]pyridazin-6-yl]amino}cyclohexanol

¹H-NMR (CDCl₃-CD₃OD (9:1)) δ: 1.23-1.39 (2H, m), 1.39-1.56 (2H, m), 1.99-2.12 (2H, m), 2.20-2.32 (2H, m), 3.60-3.77 (2H, m), 3.87 (3H, s), 6.49 (1H, d, J=9.5 Hz), 7.00 (2H, d, J=8.8 Hz), 7.58 (1H, d, J=9.5 Hz), 7.64 (1H, s), 8.03 (2H, d, J=9.2 Hz).

MS: 339 (M+H)⁺.

### Example 35

### 3-(4-Pyridinyl)-N-(3,4,5-trimethoxyphenyl)imidazo[1,2-b]pyridazin-6-amine

¹H-NMR (CDCl₃) δ: 3.81 (6H, s), 3.88 (3H, s), 6.48 (1H, brs), 6.74 (2H, s), 6.77 (1H, d, J=9.9 Hz), 6.85 (1H, d, J=9.5 Hz), 8.02 (2H, d, J=5.5 Hz), 8.03 (1H, s), 8.66 (2H, d, J=6.6 Hz).

MS: 378 (M+H)⁺.

### Example 36

### N-(2-Chlorophenyl)-3-(4-pyridinyl)imidazo[1,2-b]pyridazin-6-amine

¹H-NMR (CDCl₃) δ: 6.84 (1H, d, J=9.5 Hz), 6.92 (1H, brs), 7.05-7.13 (1H, m), 7.31-7.38 (1H, m), 7.45-7.50 (1H, m), 7.91 (1H, d, J=9.5 Hz), 8.00 (2H, d, J=6.2 Hz), 8.05 (1H, s), 8.26-8.32 (1H, m), 8.69 (2H, d, J=6.2 Hz).

MS: 322 (M+H)⁺.

### Example 37

### N-(4-Phenoxyphenyl)-3-(4-pyridinyl)imidazo[1,2-b]pyridazin-6-amine

¹H-NMR (CDCl₃-CD₃OD (9:1)) δ: 6.91 (1H, d, J=9.5 Hz), 7.01-7.16 (5H, m), 7.62 (2H, d, J=9.1 Hz), 7.78 (1H, d, J=9.5 Hz), 7.96 (1H, s), 8.08 (2H, d, J=6.2 Hz), 8.57 (2H, d, J=6.2 Hz), 7.33-7.41 (2H, m).

MS: 380 (M+H)⁺.

### Example 38

### trans-4-{[3-(4-Pyridinyl)imidazo[1,2-b]pyridazin-6-yl]amino}cyclohexanol

¹H-NMR (CDCl₃-CD₃OD (9:1)) δ: 1.24-1.58 (4H, m), 2.03-2.14 (2H, m), 2.22-2.34 (2H, m), 3.61-3.79 (2H, m), 6.62 (1H, d, J=9.9 Hz), 7.64 (1H, d, J=9.5 Hz), 7.94 (1H, s), 8.14 (2H, d, J=6.2 Hz), 8.58 (2H, d, J=5.1 Hz).

MS: 310 (M+H)⁺.

### Example 39

### (trans-4-{[3-(4-Pyridinyl)imidazo[1,2-b]pyridazin-6-yl]amino}cyclohexyl)formamide

¹H-NMR (DMSO-d₆) δ: 1.26-1.48 (4H, m), 1.81-1.98 (2H, m), 2.09-2.24 (2H, m), 3.53-3.80 (2H, m), 6.76 (1H, d, J=9.5 Hz), 7.18 (1H, d, J=6.5 Hz), 7.8 (1H, d, J=8.1 Hz), 7.98-8.09 (2H, m), 8.16 (1H, s), 8.21 (2H, d, J=6.0 Hz), 8.60 (2H, d, J=6.0 Hz).

MS: 337 (M+H)⁺.

### Example 40

### (trans-4-{[3-(4-Pyridinyl)imidazo[1,2-b]pyridazin-6-yl]amino}cyclohexyl)methanol

¹H-NMR (DMSO-d₆) δ: 1.31-1.91 (9H, m), 3.23-3.42 (2H, m), 3.90-4.02 (1H, m), 4.44 (1H, t, J=5.5 Hz), 6.88 (1H, d, J=9.5 Hz), 7.04 (1H, d, J=6.0 Hz), 7.79 (1H, d, J=9.5 Hz), 8.16 (1H, s), 8.20 (2H, d, J=6.0 Hz), 8.60 (2H, d, J=′6.0 Hz).

MS: 324 (M+H)⁺.

### Example 41

### N-(trans-4-Ethoxycyclohexyl)-3-(4-pyridinyl)imidazo[1,2-b]pyridazin-6-amine

¹H-NMR (DMSO-d₆): 1.12 (3H, t, J=7.0 Hz), 1.24-1.48 (4H, m), 1.95-2.23 (4H, m), 3.21-3.38 (1H, m), 3.49 (2H, q, J=7.0 Hz), 3.57-3.74 (1H, m), 6.76 (1H, d, J=9.5 Hz), 7.12 (1H, d, J=6.5 Hz), 7.80 (1H, d, J=9.5 Hz), 8.16 (1H, s), 8.20 (2H, d, J=1.5 Hz, 4.5 Hz), 8.61 (2H, d, J=1.5 Hz, 4.5 Hz).

MS: 338 (M+H)⁺.

### Example 42

### N-[(1S,2S)-2-(Benzyloxy)cyclohexyl]-3-(4-pyridinyl)imidazo[1,2-b]pyridazin-6-amine

MS: 400 (M+H)⁺.

### Example 43

### N-(trans-4-Methoxycyclohexyl)-3-(4-pyridinyl)imidazo[1,2-b]pyridazin-6-amine

¹H-NMR (DMSO-d₆) δ: 1.20-1.45 (4H, m), 1.94-2.27 (4H, m), 3.12-3.42 (1H, m), 3.27 (3H, s), 3.51-3.74 (1H, m), 6.76 (1H, d, J=9.9 Hz), 7.14 (1H, d, J=6.7 Hz), 7.80 (1H, d, J=9.9 Hz), 8.16 (1H, s), 8.20 (2H, dd, J=1.5H z, 4.9 Hz), 8.60 (2H, dd, J=1.5 Hz, 4.9 Hz).

MS: 324 (M+H)⁺.

### Example 44

### N-Cyclohexyl-3-(4-pyridinyl)imidazo[1,2-b]pyridazin-6-amine

¹H-NMR (DMSO-d₆) δ: 1.16-1.51 (4H, m), 1.59-1.71 (1H, m), 1.72-1.86 (2H, m), 2.03-2.16 (2H, m), 3.57-3.73 (1H, m), 6.78 (1H, d, J=9.9 Hz), 7.12 (1H, d, J=7.0 Hz), 7.79 (1H, d, J=9.9 Hz), 8.16 (1H, s), 8.21 (2H, d, J=6.2 Hz), 8.60 (2H, d, J=6.2 Hz).

MS: 294 (M+H)⁺.

### Example 45

### cis-4-{[3-(4-Pyridinyl)imidazo[1,2-b]pyridazin-6-yl]amino}cyclohexanol

¹H-NMR (DMSO-d₆) δ: 1.54-1.84 (8H, m), 3.67-3.80 (2H, m), 4.49 (1H, d, J=2.9 Hz), 6.82 (1H, d, J=9.5 Hz), 7.12 (1H, d, J=7.3 Hz), 7.79 (1H, d, J=9.9 Hz), 8.16 (1H, s), 8.20 (2H, d, J=6.2 Hz), 8.60 (2H, d, J=5.9 Hz).

MS: 310 (M+H)⁺.

### Example 46

### 3-(4-Pyridinyl)-N-(2-pyridinylmethyl)imidazo[1,2-b]pyridazin-6-amine

¹H-NMR (DMSO-d₆) δ: 4.62 (1H, d, J=5.5 Hz), 6.95 (1H, d, J=9.5 Hz), 7.24-7.31 (1H, m), 7.45 (1H, d, J=8.1 Hz), 7.72-7.79 (1H, m), 7.86 (1H, d, J=9.5 Hz), 7.90 (2H, d, J=6.2 Hz), 7.97 (1H, t, J=6.2 Hz), 8.14 (1H, s), 8.49 (2H, d, J=6.2 Hz), 8.60-8.65 (1H, m).

MS: 303 (M+H)⁺.

### Example 47

### N-Phenyl-3-(4-pyridinyl)imidazo[1,2-b]pyridazin-6-amine

¹H-NMR (DMSO-d₆) δ: 7.04 (1H, d, J=9.9 Hz), 7.06 (1H, t, J=8.4 Hz), 7.42 (2H, t, J=7.3 Hz), 7.72 (2H, d, J=7.3 Hz), 8.01 (1H, d, J=9.9 Hz), 8.15 (2 H, d, J=6.2 Hz), 8.22 (1H, s), 8.67 (2H, d, J=9.9 Hz), 9.56 (1H, s).

MS: 288 (M+H)⁺.

### Example 48

### N-Propyl-3-(4-pyridinyl)imidazo[1,2-b]pyridazin-6-amine

¹H-NMR (DMSO-d₆) δ: 1.00 (3H, t, J=7.3 Hz), 1.61-1.77 (2H, m), 3.24-3.39 (2H, m), 6.79 (1H, d, J=9.5 Hz), 7.24 (1H, t, J=5.5 Hz), 7.80 (1H, d, J=9.9 Hz), 8.17 (1H, s), 8.21 (2H, d, J=6.2 Hz), 8.61 (2H, d, J=6.2 Hz).

MS: 254 (M+H)⁺.

### Example 49

### tert-Butyl trans-4-{[3-(4-pyridinyl)imidazo[1,2-b]pyridazin-6-yl]amino}cyclohexanecarboxylate

¹H-NMR (DMSO-d₆) δ: 1.25-1.56 (4H, m), 1.42 (9H, s), 1.92-2.05 (2H, m), 2.12-2.29 (3H, m), 3.53-3.71 (1H, m), 6.76 (1H, d, J=9.5 Hz), 7.14 (1H, d, J=7.0 Hz), 7.8 (1H, d, J=9.5 Hz), 8.16 (1H, s), 8.19 (2H, d, J=6.5 Hz), 8.59 (2H, d, J=6.5 Hz).

MS: 394 (M+H)⁺.

### Example 50

### N-Bicyclo[2.2.1]hept-2-yl-3-(4-pyridinyl)imidazo[1,2-b]pyridazin-6-amine

¹H-NMR (DMSO-d₆) δ: 0.94-1.06 (1H, m), 1.22-1.42 (3H, m), 1.46-1.70 (3H, m), 2.04-2.18 (1H, m), 2.20-2.30 (1H, m), 2.64-2.73 (1H, m), 3.93-4.07 (1H, m), 6.85 (1H, d, J=9.5 Hz), 7.27 (1H, d, J=5.5 Hz), 7.80 (1H, d, J=9.5 Hz), 8.15 (1H, s), 8.21 (2H, d, J=6.2 Hz), 8.61 (2H, d, J=6.2 Hz).

MS: 306 (M+H)⁺.

### Example 51

### N-[(2R)-Bicyclo[2.2.1]hept-2-yl]-3-(4-pyridinyl)imidazo[1,2-b]pyridazin-6-amine

¹H-NMR (DMSO-d₆) δ: 1.13-1.26 (2H, m), 1.28-1.68 (5H, m), 1.73-1.86 (1H, m), 2.25-2.33 (1H, m), 2.41-2.47 (1H, m), 3.53-3.63 (1H, m), 6.76 (1H, d, J=9.5 Hz), 7.13 (1H, d, J=5.9 Hz), 7.80 (1H, d, J=9.5 Hz), 8.18 (1H, s), 8.26 (2H, d, J=6.2 Hz), 8.60 (2H, d, J=6.2 Hz).

MS: 306 (M+H)⁺.

### Example 52

### N-(5-Fluoro-2-methylphenyl)-3-(4-pyridinyl)imidazo[1,2-b]pyridazin-6-amine

MS: 320 (M+H)⁺.

### Example 53

### N-Methoxy-4-methyl-3-{[3-(4-pyridinyl)imidazo[1,2-b]pyridazin-6-yl]amino}benzamide

MS: 375 (M+H)⁺.

### Example 54

### tert-Butyl 3-{[3-(4-pyridinyl)imidazo[1,2-b]pyridazin-6-yl]amino}-1-piperidinecarboxylate

¹H-NMR (DMSO-d₆) δ: 1.06-2.01 (14H, m), 1.39 (3H, s), 3.74 (2H, brs), 6.84 (1H, d, J=9.6 Hz), 7.13 (1H, d, J=6.6 Hz), 7.84 (1H, d, J=9.6 Hz), 8.17-8.21 (3H, m), 8.58-8.61 (2H, m).

MS: 395 (M+H)⁺, 417 (M+Na)⁺.

### Example 55

### 4-{[3-(4-Pyridinyl)imidazo[1,2-b]pyridazin-6-yl]amino}-1-adamantanol

¹H-NMR (DMSO-d₆) δ1.35-1.45 (2H, m), 1.65-2.07 (9H, m), 2.26-2.33 (2H, m), 3.90 (1H, bs), 4.51 (1H, s), 6.96 (1H, d), 7.11 (1H, d), 7.81 (1H, d), 8.16 (1H, s), 8.19 (2H, d), 8.58 (2H, d).

MS: 362 (M+H)⁺.

### Example 56

### 4-{[3-(4-Pyridinyl)imidazo[1,2-b]pyridazin-6-yl]amino}-1-adamantanol

¹H-NMR (DMSO-d₆) δ: 1.38-2.18 (11H, m), 2.35-2.42 (2H, m), 3.81 (1H, b s), 4.44 (1H, s), 6.96 (1H, d), 7.11 (1H, d), 7.80 (1H, d), 8.16 (1H, s), 8.19 (2H, d), 8.58 (2H, d).

MS: 362 (M+H)⁺.

### Example 57

### 3-(4-Pyridinyl)-N-(2,3,6-trifluorobenzyl)imidazo[1,2-b]pyridazin-6-amine

¹H-NMR (DMSO-d₆) δ: 4.66 (2H, d), 6.82 (1H, d), 7.15-7.28 (1H, m), 7.38-7.58 (1H, m), 7.70-7.81 (1H, m), 7.85 (1H, d), 8.15 (2H, d), 8.17 (1H, s), 8.61 (2H, d).

MS: 356 (M+H)⁺.

### Example 58

### 2-(3-{[3-(4-Pyridinyl)imidazo[1,2-b]pyridazin-6-yl]amino}benzyl)-1H-isoindole-1,3(2H)-dione

MS: 447 (M+H)⁺.

### Example 59

### N-(5-Methoxyadamantan-2-yl)-3-(4-pyridinyl)imidazo[1,2-b]pyridazin-6-amine

¹H-NMR (DMSO-d₆) δ: 1.38-1.59 (2H, m), 1.62-2.22 (9H, m), 2.34-2.56 (2H, m), 3.16 (3H, d), 3.80-4.00 (1H, m), 6.96 (1H, d), 7.08-7.20 (1H, m), 7.81 (1H, d), 8.16 (1H, s), 8.19 (2H, d), 8.59 (2H, d).

MS: 376 (M+H)⁺.

### Example 60

### N-(4-Methoxyadamantan-1-yl)-3-(4-pyridinyl)imidazo[1,2-b]pyridazin-6-amine

¹H-NMR (DMSO-d₆) δ: 1.40-1.48 (2H, m), 1.91-2.26 (11H, m), 3.30 (3H, s), 3.41 (1H, s), 6.79 (1H, d), 6.84 (1H, s), 7.77 (1H, d), 8.12 (1H, s), 8.17 (2H, d), 8.60 (2H, d).

MS: 376 (M+H)⁺.

### Example 61

### N-(4-Fluoroadamantan-1-yl)-3-(4-pyridinyl)imidazo[1,2-h]pyridazin-6-amine

¹H-NMR (CDCl₃) δ: 0.82-2.63 (13H, m), 4.61-4.85 (1H, m), 6.40-6.60 (1H, m), 7.74 (1H, dd), 8.02-8.60 (4H, m), 8.67 (2H, dd).

MS: 364 (M+H)⁺.

### Example 62

### 8-Methyl-N-[(1S,2R)-2-methylcyclohexyl]-3-(4-pyridinyl) imidazo[1,2-b]pyridazin-6-amine

¹H-NMR (DMSO-d₆) δ: 0.92 (3H, d), 1.19-1.98 (8H, m), 2.28 (3H, s), 2.39-2.55 (1H, m), 3.95-4.13 (1H, m), 5.77 (1H, d), 7.71 (1H, s), 8.14 (1H, s), 8.19 (2H, d), 8.59 (2H, d).

MS: 344 (M+Na)⁺.

### Example 63

### 8-Methyl-N-[2-methyl-5-(tetrahydro-2H-pyran-2-yloxy)phenyl]-3-(4-pyridinyl)imidazo[1,2-b]pyridazin-6-amine

¹H-NMR (DMSO-d₆) δ: 1.40-1.89 (6H, m), 2.14 (3H, s), 2.43 (3H, s), 3.42-3.55 (1H, m), 3.67-3.83 (1H, m), 5.42 (1H, t), 6.93 (1H, dd), 7.16 (1H, d), 7.26 (1H, d), 7.87 (1H, d), 7.89 (2H, d), 8.07 (1H, s), 8.19 (1H, s), 8.36 (2H, d).

MS: 416 (M+Na)⁺.

### Example 64

### 5-{[8-Methyl-3-(4-pyridinyl)imidazo[1,2-b]pyridazin-6-yl]amino}-2-adamantanol

¹H-NMR (CDCl₃) δ: 1.52-2.85 (13H, m), 2.25 (3H, s), 3.91 (0.37H, t), 4.05 (0.63H, t), 4.12 (0.63H, s), 4.15 (0.37H, s), 7.58 (1H, s), 7.95 (0.63H, s), 8.00 (0.37H, s), 8.09 (1.26H, d), 8.24 (0.74H, d), 8.66 (2H, d).

MS: 376 (M+H)⁺.

### Example 65

### trans-4-{[3-(2-Chloro-4-pyridinyl)-8-methylimidazo[1,2-b]pyridazin-6-yl]amino}cyclohexanol

¹H-NMR (DMSO-d₆) δ: 1.35-1.51 (4H, m), 1.86-1.97 (2H, m), 2.03-2.21 (2H, m), 2.23 (3H, s), 3.40-3.55 (1H, m), 3.62-3.79 (1H, m), 4.62 (1H, s), 6.20 (1H, d), 7.73 (1H, d), 8.06 (1H, dd), 8.25 (1H, s), 8.39 (1H, d), 8.54 (1H, s).

MS: 358 (M+H)⁺.

### Example 66

### trans-4-{[8-Methoxy-3-(4-pyridinyl)imidazo[1,2-b]pyridazin-6-yl]amino}cyclohexanol

¹H-NMR (DMSO-d₆) δ: 1.20-1.82 (8H, m), 2.10 (1H, d), 2.30 (1H, d), 3.59-3.66 (1H, m), 3.70-3.83 (1H, m), 4.03 (3H, s), 5.79 (1H, s), 7.90 (1H, s), 8.06 (2H, d), 8.64 (2H, d).

MS: 340 (M+H)⁺.

### Example 67

### N-(cis-4-Phenoxycyclohexyl)-3-(4-pyridinyl)imidazo[1,2-b]pyridazin-6-amine

MS: 386 (M+H)⁺.

### Example 68

### N-[cis-4-(Phenoxymethyl)cyclohexyl]-3-(4-pyridinyl)imidazo[1,2-b]pyridazin-6-amine

MS: 400 (M+H)⁺.

### Example 69

### 3-(4-Pyridinyl)-N-[cis-4-(2,2,2-trifluoroethoxy)cyclohexyl]imidazo[1,2-b]pyridazin-6-amine

MS: 392 (M+H)⁺.

### Example 70

### N-(4-Isopropoxycyclohexyl)-3-(4-pyridinyl)imidazo[1,2-b]pyridazin-6-amine

¹H-NMR (DMSO-d₆) δ: 1.09 (6H, d, J=6.0 Hz), 1.57-1.85 (8H, m), 3.55-3.60 (1H, m), 3.63-3.72 (1H, m), 3.72-3.81 (1H, m), 6.80 (1H, d, J=9.6 Hz), 7.12 (1H, d, J=6.8 Hz), 7.79 (1H, d, J=9.6 Hz), 8.16 (1H, s), 8.19-8.21 (2H, m), 8.59-8.61 (2H, m).

MS: 352 (M+H)⁺.

### Example 71

### N-[trans-4-(Benzyloxy)cyclohexyl]-3-(4-pyridinyl)imidazo[1,2-b]pyridazin-6-amine

MS: 400 (M+H)⁺.

### Example 72

### N-(4-Methoxy-2-methylphenyl)-3-(4-pyridinyl)imidazo[1,2-b]pyridazin-6-amine

MS: 332 (M+H)⁺.

### Example 73

### N-[trans-4-(2-Methoxyethoxy)cyclohexyl]-3-(4-pyridinyl) imidazo[1,2-b]pyridazin-6-amine

¹H-NMR (DMSO-d₆) δ: 1.23-1.45 (4H, m), 2.01-2.22 (4H, m), 3.27 (3H, s), 3.31-3.48 (3H, m), 3.53-3.72 (3H, m), 6.76 (1H, d, J=9.6 Hz), 7.12 (1H, d, J=6.4 Hz), 7.79 (1H, d, J=9.6 Hz), 8.16 (1H, s), 8.18-8.23 (2H, m), 8.59-8.63 (2H, m).

MS: 390 (M+Na)⁺.

### Example 74

### N-(1,1-Dioxidotetrahydro-2H-thiopyran-4-yl)-3-(4-pyridinyl)imidazo[1,2-b]pyridazin-6-amine

MS: 366 (M+Na)⁺.

### Example 75

### N-[3-Methoxy-5-(trifluoromethyl)phenyl]-3-(4-pyridinyl) imidazo[1,2-b]pyridazin-6-amine

MS: 386 (M+H)⁺.

### Example 76

### 3-(4-Pyridinyl)-N-(1,2,3,4-tetrahydro-1-naphthalenyl) imidazo[1,2-b]pyridazin-6-amine

MS: 342 (M+H)⁺.

### Example 77

### 3-(4-Pyridinyl)-N-[(1R,2S,4R)-1,7,7-trimethylbicyclo[2.2.1]hept-2-yl]imidazo[1,2-b]pyridazin-6-amine

MS: 348 (M+H)⁺.

### Example 78

### 3-(4-Pyridinyl)-N-[(1R,2R,4R)-1,7,7-trimethylbicyclo[2.2.1]hept-2-yl]imidazo[1,2-b]pyridazin-6-amine

MS: 348 (M+H)⁺.

### Example 79

### N-(trans-4-Ethoxycyclohexyl)-8-methyl-3-(4-pyridinyl) imidazo[1,2-b]pyridazin-6-amine

¹H-NMR (CDCl₃) δ: 1.25 (3H, t, J=7.2 Hz), 1.26-1.61 (4H, m), 2.09-2.42 (4H, m), 2.24 (3H, s), 3.27-3.43 (1H, m), 3.57 (2H, q, J=7.2 Hz), 3.79-3.96 (1H, m), 4.19 (1H, d, J=6.4 Hz), 7.56-7.58 (1H, m), 7.98 (1H, s), 8.08-8.12 (2H, m), 8.62-8.66 (2H, m).

MS: 352 (M+H)⁺.

### Example 80

### tert-Butyl[(trans-4-{[3-(4-pyridinyl)imidazo[1,2-b]pyridazin-6-yl]amino}cyclohexyl)oxy]acetate

MS: 446 (M+Na)⁺.

### Example 81

### 2-Methyl-N-[3-(4-pyridinyl)imidazo[1,2-b]pyridazin-6-yl]-1,2,3,4-tetrahydro-7-isoquinolinamine

¹H-NMR (DMSO-d₆) δ: 2.37 (3H, s), 2.62 (2H, t, J=6.0 Hz), 2.80 (2H, t, J=6.0 Hz), 3.51 (2H, s), 7.00 (1H, d, J=9.6 Hz), 7.12 (1H, d, J=8.0 Hz), 7.29 (1H, dd, J=8.4, 2.2 Hz), 7.62 (1H, d, J=2.2 Hz), 7.98 (1H, d, J=9.6 Hz), 8.13-8.15 (2H, m), 8.20 (1H, s), 8.64-8.66 (2H, m), 9.45 (1H, s).

MS: 357 (M+H)⁺.

### Example 82

### N-(trans-4-Fluorocyclohexyl)-3-(4-pyridinyl)imidazo[1,2-b]pyridazin-6-amine

¹H-NMR (DMSO-d₆) δ: 1.40-1.51 (2H, m), 1.69-1.82 (2H, m), 2.12-2.23 (2H, m), 2.28-2.36 (2H, m), 3.81-3.90 (1H, m), 4.52-4.74 (2H, m), 6.59 (1H, d, J=9.6 Hz), 7.77 (1H, d, J=9.6 Hz), 8.08 (1H, s), 8.21-8.23 (2H, m), 8.64-8.68 (2H, m).

MS: 312 (M+H)⁺.

### Example 83

### N,N-Dimethyl-2-[(trans-4-{[3-(4-pyridinyl)imidazo[1,2-b]pyridazin-6-yl]amino}cyclohexyl)oxy]acetamide

MS: 417 (M+Na)⁺.

### Example 84

### 6-tert-Butoxy-3-(4-pyridinyl)imidazo[1,2-b]pyridazine

MS: 269 (M+H)⁺.

### Example 85

To a solution of trans-N-[3-(4-pyridinyl)imidazo[1,2-b]pyridazin-6-yl]-1,4-cyclohexanediamine trihydrochloride (7 mg) in pyridine (0.5 ml) was added acetic anhydride (2 μl). After stirring for 1 hour, the mixture was evaporated in vacuo. The residue was purified by column chromatography on silica gel eluting with chloroform/methanol (100:2 to 10:1) to give N-(trans-4-{[3-(4-pyridinyl)imidazo[1,2-b]pyridazin-6-yl]amino}cyclohexyl)acetamide as an yellow solid (2 mg).

¹H-NMR (DMSO-d₆) δ: 1.07-1.49 (4H, m), 1.81 (3H, s), 1.85-2.00 (2H, m), 2.12-2.22 (2H, m), 3.51-3.70 (2H, m), 6.77 (1H, d, J=9.5 Hz), 7.20 (1H, d, J=6.5 Hz), 7.78-7.83 (2H, m), 8.16 (1H, s), 8.21 (2H, d, J=6.0 Hz), 8.60 (2H, d, J=6.0 Hz).

MS: 351 (M+H)⁺.

### Example 86

To a stirred mixture of trans-4-[(3-iodoimidazo[1,2-b]pyridazin-6-yl)amino]cyclohexanol (40 mg) and phenylboronic acid (54.5 mg) in 1,2-dimethoxyethane (0.45 ml) was added 2 M sodium carbonate aqueous solution (0.448 mL) at ambient temperature. Bis(triphenylphosphine) palladium (II) chloride (4.7 mg) was then added to the mixture at ambient temperature. After addition, the resulting mixture was stirred at 85° C. for 1 hour. The reaction mixture was cooled to ambient temperature and diluted with ethyl acetate/water (20 mL:20 mL). The resulting mixture was acidified with 1M HCl aqueous solution to pH 2 and extracted with ethyl acetate. The aqueous phase was then neutralized by the addition of 2M NaOH aqueous solution to pH 8. The resulting solution was extracted with ethyl acetate three times, the organic layers were combined, dried over magnesium sulfate, and concentrated in vacuo. The residue was purified by silica gel column chromatography eluting with chloroform/methanol (20:1) to give trans-4-[(3-phenylimidazo[1,2-b]pyridazin-6-yl)amino]cyclohexanol.

¹H-NMR (DMSO-d₆) δ: 1.17-1.41 (4H, m), 1.83-1.96 (2H, m), 2.04-2.18 (2H, m), 3.39-3.65 (2H, m), 4.61 (1H, d, J=4.4 Hz), 6.67 (1H, d, J=9.5 Hz), 6.96 (1H, d, J=6.6 Hz), 7.32 (1H, t, J=7.2 Hz), 7.45 (2H, t, J=7.9 Hz), 7.74 (1H, d, J=9.9 Hz), 7.88 (1H, s), 8.20 (2H, d, J=7.7 Hz).

MS: 309 (M+H)⁺.

The following compounds were obtained in a similar manner to that of Example 86.

### Example 87

### trans-4-({3-[3-(Trifluoromethoxy)phenyl]imidazo[1,2-b]pyridazin-6-yl}amino)cyclohexanol

¹H-NMR (DMSO-d₆) δ: 1.22-1.37 (4H, m), 1.83-1.95 (2H, m), 2.04-2.16 (2H, m), 3.42-3.66 (2H, m), 4.62 (1H, d, J=4.0 Hz), 6.71 (1H, d, J=9.5 Hz), 7.04 (1H, d, J=7.0 Hz), 7.30 (1H, d, J=9.5 Hz), 7.58 (1H, d, J=8.1 Hz), 7.77 (1H, d, J=9.5 Hz), 8.04 (1H, s), 8.09 (1H, d, J=7.7 Hz), 8.48 (1H, s).

MS: 393 (M+H)⁺.

### Example 88

### trans-4-({3-[3-(Benzyloxy)phenyl]imidazo[1,2-b]pyridazin-6-yl}amino)cyclohexanol

¹H-NMR (DMSO-d₆) δ: 1.18-1.41 (4H, m), 1.83-1.94 (2H, m), 2.05-2.17 (2H, m), 3.41-3.68 (2H, m), 4.58 (1H, d, J=4.0 Hz), 5.19 (2H, s), 6.67 (1H, d, J=9.9 Hz), 6.92-7.01 (2H, m), 7.34 (1H, d, J=8.1 Hz), 7.42 (3H, t, J=7.7 Hz), 7.50 (2H, d, J=7.0 Hz), 7.71-7.80 (2H, m), 7.89-7.94 (2H, m).

MS: 415 (M+H)⁺.

### Example 89

### trans-4-{[3-(3-Biphenylyl)imidazo[1,2-b]pyridazin-6-yl]amino}cyclohexanol

¹H-NMR (DMSO-d₆) δ: 1.10-1.34 (4H, m), 1.74-1.85 (2H, m), 2.02-2.13 (2H, m), 3.37-3.71 (2H, m), 4.54 (1H, d, J=3.7 Hz), 6.69 (1H, d, J=9.9 Hz), 6.93 (1H, d, J=7.3 Hz), 7.37-7.45 (5H, m), 7.48-7.64 (4H, m), 7.75 (1H, d, J=9.2 Hz), 7.97 (1H, s), 8.11 (1H, d, J=7.7 Hz), 8.51 (1H, s).

MS (ESI): 385 (M+H)⁺.

### Example 90

### 4-{6-[(trans-4-Hydroxycyclohexyl)amino]imidazo[1,2-b]pyridazin-3-yl}phenol

MS: 325 (M+H)⁺.

### Example 91

### trans-4-{[3-(3-Methoxyphenyl)imidazo[1,2-b]pyridazin-6-yl]amino}cyclohexanol

¹H-NMR (DMSO-d₆) δ: 1.19-1.36 (4H, m), 1.82-1.94 (2H, m), 2.05-2.16 (2H, m), 3.40-3.67 (2H, m), 3.84 (3H, s), 4.60 (1H, d, J=4.4 Hz), 6.67 (1H, d, J=9.5 Hz), 6.86-6.97 (2H, m), 7.35 (1H, t, J=8.1 Hz), 7.68-7.73 (1H, m), 7.73 (1H, d, J=9.5 Hz), 7.85-7.93 (1H, m), 7.89 (1H, s).

MS: 339 (M+H)⁺.

### Example 92

### trans-4-({3-[3-(Trifluoromethyl)phenyl]imidazo[1,2-h]pyridazin-6-yl}amino)cyclohexanol

¹H-NMR (DMSO-d₆) δ: 1.18-1.37 (4H, m), 1.79-1.95 (2H, m), 2.01-2.14 (2H, m), 3.04-3.67 (2H, m), 4.62 (1H, d, J=4.0 Hz), 6.72 (1H, d, J=9.5 Hz), 7.02 (1H, d, J=7.3 Hz), 7.64-7.72 (2H, m), 7.77 (1H, d, J=9.5 Hz), 8.05 (1H, s), 8.28-8.35 (1H, m), 8.83 (1H, brs).

MS: 377 (M+H)⁺.

### Example 93

### 3-{6-[(trans-4-Hydroxycyclohexyl)amino]imidazo[1,2-b]pyridazin-3-yl}phenol

¹H-NMR (DMSO-d₆) δ: 1.15-1.43 (4H, m), 1.81-1.94 (2H, m), 2.06-2.18 (2H, m), 3.41-3.65 (2H, m), 4.60 (1H, d, J=4.0 Hz), 6.65 (1H, d, J=9.5 Hz), 6.73 (1H, d, J=8.1 Hz), 6.91 (1H, d, J=7.0 Hz), 7.22 (1H, t, J=7.3 Hz), 7.59 (1H, d, J=7.7 Hz), 7.64 (1H, s), 7.71 (1H, d, J=9.5 Hz), 7.79 (1H, s), 9.45 (1H, s).

MS: 325 (M+H)⁺.

### Example 94

### trans-4-{[3-(3-Pyridinyl)imidazo[1,2-b]pyridazin-6-yl]amino}cyclohexanol

¹H-NMR (DMSO-d₆) δ: 1.18-1.40 (4H, m), 1.84-1.95 (2H, m), 2.04-2.15 (2H, m), 3.41-3.66 (2H, m), 4.61 (1H, d, J=4.4 Hz), 6.71 (1H, d, J=9.5 Hz), 7.04 (1H, d, J=7.0 Hz), 7.44-7.52 (1H, m), 7.77 (1H, d, J=9.5 Hz), 8.01 (1H, s), 8.48-8.59 (2H, m), 9.38 (1H, d, J=1.8 Hz).

MS: 310 (M+H)⁺.

### Example 95

### 4-{6-[(trans-4-Hydroxycyclohexyl)amino]imidazo[1,2-b]pyridazin-3-yl}-2-methoxyphenol

¹H-NMR (CDCl₃-CD₃OD (9:1)) δ: 1.17-1.56 (4H, m), 1.96-2.10 (2H, m), 2.13-2.29 (2H, m), 3.57-3.82 (2H, m), 3.97 (3H, s), 6.48 (1H, d), 6.97 (1H, d), 7.53-7.67 (4H, m).

MS: 355 (M+H)⁺.

### Example 96

### trans-4-{[3-(3-Bromophenyl)imidazo[1,2-b]pyridazin-6-yl]amino}cyclohexanol

¹H-NMR (CDCl₃-CD₃OD (9:1)) δ: 1.23-1.40 (2H m), 1.47-1.64 (2H, m), 1.99-2.11 (2H, m), 2.23-2.34 (2H, m), 3.60-3.81 (2H, m), 6.54 (1H, d, J=9.5 Hz), 7.31 (1H, d, J=7.7 Hz), 7.42-7.49 (1H, m), 7.61 (1H, d, J=9.9 Hz), 7.75 (1H, s), 7.86-7.92 (1H, m), 8.52-8.56 (1H, m).

MS: 387 (M+H)⁺.

### Example 97

### trans-4-{[3-(2-Pyridinyl)imidazo[1,2-b]pyridazin-6-yl]amino}cyclohexanol

¹H-NMR (CDCl₃-CD₃OD (95:5)) δ: 1.07-1.65 (4H, m), 2.03-2.16 (2H, m), 2.25-2.40 (2H, m), 3.64-3.88 (2H, m), 6.47 (1H, d), 7.17-7.36 (1H, m), 7.68-7.84 (2H, m), 8.29 (1H, s), 8.60-8.73 (2H, m).

MS: 310 (M+H)⁺.

### Example 98

A mixture of 6-chloro-3-(4-pyridinyl)imidazo[1,2-b]pyridazine (20 mg) and morpholine (1.0 mL) was stirred at 110° C. for 2 hours. Evaporation of the volatile components gave a residue, which was purified by silica gel column chromatography eluting with chloroform/methanol (20:1) to give 6-(4-morpholinyl)-3-(4-pyridinyl)imidazo[1,2-b]pyridazine (6.63 mg).

¹H-NMR (CDCl₃) δ: 3.50-3.61 (4H, m), 3.85-3.96 (4H, m), 6.92 (1H, d, J=9.9 Hz), 7.85 (1H, d, J=9.9 Hz), 8.02 (2H, d, J=6.2 Hz), 8.05 (1H, s), 8.69 (2H, d, J=6.2 Hz), 2.99-3.09 (1H, m), 3.13-3.25 (1H, m), 3.51-3.61 (5H, m), 3.62-3.80 (2H, m), 3.82-3.99 (5H, m), 4.18-4.29 (1H, m), 4.60-4.71 (1H, m), 7.09 (1H, s), 7.97-8.04 (3H, m), 8.67 (2H, d, J=6.2 Hz).

MS: 282.1 (M+H)⁺.) as a yellow powder and further elution to give 6,7-di-4-morpholinyl-3-(4-pyridinyl)imidazo[1,2-b]pyridazine (16.7 mg. MS: 367 (M+H)⁺.) as a yellow powder.

### Example 99

To a stirred mixture of 6-chloro-3-(4-pyridinyl)imidazo[1,2-b]pyridazine (100 mg) and [(1R)-1-phenylethyl]amine (157 mg) in toluene (5 ml) were added sodium tert-butoxyde (187 mg), (R)-2,2′-Bis(diphenylphosphino)-1,1′-binaphthyl (=(R)-BINAP, 24.3 mg), and tris(dibenzylideneacetone)dipalladium chloroform complex (═Pd₂(dba)₃.CHCl₃, 13.5 mg) at ambient temperature. The reaction was stirred at 110° C. for 3 hours. After all starting material had been consumed, as judged by TLC plate, the reaction mixture was cooled to ambient temperature and diluted with ethyl acetate/water (10 mL:10 mL). The resulting mixture was acidified with 1 M HCl aqueous solution to pH 2 and extracted with ethyl acetate. The aqueous phase was then adjusted to pH 8 with 2M NaOH aqueous solution. The resulting solution was extracted with ethyl acetate three times, the organic layers were combined, dried over magnesium sulfate, and concentrated in vacuo. The residue was purified by silica gel column chromatography eluting with chloroform/methanol (40:1 to 20:1) to give N-[(1R)-1-phenylethyl]-3-(4-pyridinyl) imidazo[1,2-b]pyridazin-6-amine (101.3 mg).

¹H-NMR (DMSO-d₆) δ: 1.51 (3H, d, J=7.0 Hz), 4.81-4.93 (1H, m), 6.90 (1H, d, J=9.9 Hz), 7.20 (1H, t, J=7.3 Hz), 7.37 (2H, t, J=7.3 Hz), 7.46 (2H, d, J=6.6 Hz), 7.78 (1H, d, J=5.9 Hz), 7.82 (1H, d, J=9.5 Hz), 7.84 (2H, d, J=6.2 Hz), 8.09 (1H, s), 8.51 (2H, d, J=6.2 Hz).

MS: 316 (M+H)⁺.

[α]_{D}=+412° (c=0.50, methanol, 24° C.).

The following compounds were obtained in a similar manner to that of Example 99.

### Example 100

### N-[2-(Benzyloxy)phenyl]-3-(4-pyridinyl)imidazo[1,2-b]pyridazin-6-amine

MS: 394 (M+H)⁺.

### Example 101

### N-3-Pyridinyl-3-(4-pyridinyl)imidazo[1,2-b]pyridazin-6-amine

¹H-NMR (DMSO-d₆) δ: 7.06 (1H, d, J=9.5 Hz), 7.45 (1H, dd, J=5.0, 8.0 Hz), 8.06 (1H, d, J=9.5 Hz), 8.09 (2H, d, J=6.0 Hz), 8.22 (1H, s), 8.16-8.30 (2H, m), 8.66 (2H, d, J=6.0 Hz), 8.84 (1H, d, J=2.5 Hz), 9.77 (1H, bs).

MS: 289 (M+H)⁺.

### Example 102

### N-Benzyl-3-(4-pyridinyl)imidazo[1,2-b]pyridazin-6-amine

¹H-NMR (DMSO-d₆) δ: 4.53 (2H, d, J=5.5 Hz), 6.88 (1H, d, J=9.5 Hz), 7.20-7.48 (5H, m), 7.84 (1H, d, J=9.5 Hz), 7.85 (1H, s), 7.98 (2H, d, J=6.5 Hz), 8.13 (1H, s), 8.52 (2H, d, J=6.5 Hz).

MS: 302 (M+H)⁺.

### Example 103

### trans-4-[(2,3-Diphenylimidazo[1,2-b]pyridazin-6-yl)amino]cyclohexanol

MS: 385 (M+H)⁺.

### Example 104

### Ethyl 4-{[3-(4-Pyridinyl)imidazo[1,2-b]pyridazin-6-yl]amino}benzoate

MS: 360 (M+H)⁺.

### Example 105

### N-[2-Methyl-5-(tetrahydro-2H-pyran-2-yloxy)phenyl]-3-(4-pyridinyl)imidazo[1,2-b]pyridazin-6-amine

¹H-NMR (DMSO-d₆) δ: 1.40-1.96 (6H, m), 2.24 (3H, s), 3.40-3.52 (1H, m), 3.68-3.81 (1H, m), 5.38 (1H, t, J=4.0 Hz), 6.81 (1H, dd, J=2.5, 8.5 Hz), 7.15 (1H, d, J=9.5 Hz), 7.19 (1H, d, J=7.0 Hz), 7.50 (1H, d, J=2.5 Hz), 8.00 (1H, d, J=9.5 Hz), 8.09 (2H, d, J=6.0 Hz), 8.25 (1H, s), 8.51 (2H, d, J=6.0 Hz), 8.65 (1H, bs).

MS: 402 (M+H)⁺.

### Example 106

### N,3-Di-4-pyridinylimidazo[1,2-b]pyridazin-6-amine

¹H-NMR (DMSO-d₆) δ: 7.08 (1H, d, J=9.5 Hz), 7.66 (2H, d, J=6.0 Hz), 8.11 (1H, d, J=9.5 Hz), 8.12 (2H, d, J=6.0 Hz), 8.27 (1H, s), 8.47 (2H, d, J=6.0 Hz), 8.73 (2H, d, J=6.0 Hz), 10.04 (1H, bs).

MS: 289 (M+H)⁺.

### Example 107

### N,N-Dimethyl-N′-[3-(4-pyridinyl)imidazo[1,2-b]pyridazin-6-yl]-1,4-benzenediamine

¹H-NMR (DMSO-d₆) δ: 2.90 (6H, s), 6.82 (2H, d, J=9.0 Hz), 6.95 (1H, d, J=9.5 Hz), 7.52 (2H, d, J=9.0 Hz), 7.92 (1H, d, J=9.5 Hz), 8.16 (2H, d, J=6.5 Hz), 8.18 (1H, s), 8.64 (2H, d, J=6.5 Hz), 9.20 (1H, bs).

MS: 331 (M+H)⁺.

### Example 108

### N-[(1S)-1-Phenylethyl]-3-(4-pyridinyl)imidazo[1,2-b]pyridazin-6-amine

¹H-NMR (DMSO-d₆) δ: 1.51 (3H, d, J=7.0 Hz), 4.81-4.93 (1H, m), 6.90 (1H, d, J=9.9 Hz), 7.20 (1H, t, J=7.3 Hz), 7.37 (2H, t, J=7.3 Hz), 7.46 (2H, d, J=6.6 Hz), 7.78 (1H, d, J=5.9 Hz), 7.82 (1H, d, J=9.5 Hz), 7.84 (2H, d, J=6.2 Hz), 8.09 (1H, s), 8.51 (2H, d, J=6.2 Hz).

MS: 316 (M+H)⁺.

[α](D)=−389° (c=0.50, methanol, 24° C.).

### Example 109

### N-[4-(Benzyloxy)phenyl]-3-(4-pyridinyl)imidazo[1,2-b]pyridazin-6-amine

¹H-NMR (DMSO-d₆) δ: 5.15 (2H, s), 6.97 (1H, d), 7.09 (2H, d, J=9.0 Hz), 7.32-7.53 (5H, m), 7.61 (2H, d, J=9.0 Hz), 7.96 (1H, d, J=9.5 Hz), 8.14 (2H, d, J=6.0 Hz), 8.19 (1H, s), 8.64 (2H, d, J=6.0 Hz), 9.38 (1H, bs).

MS: 394 (M+H)⁺.

### Example 110

### N-[3-(Benzyloxy)phenyl]-3-(4-pyridinyl)imidazo[1,2-b]pyridazin-6-amine

¹H-NMR (DMSO-d₆) δ: 5.09 (2H, s), 6.72 (1H, dd, J=8.0, 8.0 Hz), 7.03 (1H, d, J=9.5 Hz), 7.17 (1H, d, J=8.0 Hz), 7.29 (1H, d, J=8.0 Hz), 7.32-7.50 (5H, m), 7.56 (1H, t, J=2.6 Hz), 8.02 (1H, d, J=9.5 Hz), 8.15 (2H, d, J=6.0 Hz), 8.21 (1H, s), 8.59 (2H, d, J=6.0 Hz), 9.57 (1H, bs).

MS: 394 (M+H)⁺.

### Example 111

To a suspension of trans-4-{[3-(4-pyridinyl)imidazo[1,2-b]pyridazin-6-yl]amino}cyclohexanol (60 mg) in dichloromethane (1.2 ml) was added diethylaminosulfur trifluoride (=DAST, 51 μl). After stirring for 1 hour, the mixture was poured into saturated NaHCO₃ aqueous solution and extracted with 10% methanol in chloroform, dried over sodium sulfate and evaporated in vacuo. The residue was purified by column chromatography on silica gel eluting with chloroform/methanol (100:2 to 10:1) to give N-3-cyclohexen-1-yl-3-(4-pyridinyl)imidazo[1,2-b]pyridazin-6-amine as an pale yellow solid (35 mg).

¹H-NMR (DMSO-d₆) δ: 1.48-1.69 (1H, m), 1.94-2.20 (4H, m), 2.46-2.53 (1H, m), 3.87-3.95 (1H, m), 5.67-5.79 (2H, m), 6.81 (1H, d, J=9.5 Hz), 7.18 (1H, d, J=7.0 Hz), 7.81 (1H, d, J=9.5 Hz), 8.16 (1H, s), 8.18-8.21 (2H, m), 8.58-8.61 (2H, m).

MS: 292 (M+H)⁺.

### Example 112

To a mixture of 6-chloro-3-(4-pyridinyl)imidazo[1,2-b]pyridazine (80 mg) and cyclopropylamine (72 μL) in toluene (6.9 mL) was added tris(dibenzylidenacetone) dipalladium chloroform complex (═Pd₂ dba₃.CHCl₃, 10.8 mg), 2,2′-Bis(diphenylphosphino)-1,1′-binaphthyl (=BINAP, 19.4 mg), and sodium tert-butoxide (150.0 mg), which was subjected to microwave irradiation at 110° C. for 2 hours. The resultant was partitioned between dichloromethane and water. Organic phase was separated, washed with brine and dried over sodium sulfate. Evaporation of the solvent gave a residue, which was purified by column chromatography on silica gel to give N-cyclopropyl-3-(4-pyridinyl)imidazo[1,2-b]pyridazin-6-amine (26.1 mg) as a yellow powder.

¹H-NMR (DMSO-d₆) δ: 0.48-0.59 (2H, m), 0.78-0.91 (2H, m), 2.60-2.79 (1H, m), 6.75 (1H, d, J=9.7 Hz), 7.52 (1H, brs), 7.83 (1H, d, J=10.0 Hz), 8.22 (1H, s), 8.34 (2H, dd, J=1.5, 4.5 Hz), 8.62 (21-1, dd, J=1.5, 4.5 Hz).

MS: 252 (M+H)⁺.

### Example 113

To a suspension of trans-N-[3-(4-pyridinyl)imidazo[1,2-b]pyridazin-6-yl]-1,4-cyclohexanediamine trihydrochloride (20 mg) in dichloromethane (0.4 ml) was added methanesulfonyl chloride (10 μl) and triethylamine (26 μl). After stirring at ambient temperature for 2 hours, the mixture was poured into saturated NaHCO₃ aqueous solution and extracted with 10% methanol in chloroform. The organic layer was washed with brine, dried over sodium sulfate, and evaporated in vacuo. The resulting precipitates were collected by filtration to give N-(trans-4-{[3-(4-pyridinyl)imidazo[1,2-b]pyridazin-6-yl]amino}cyclohexyl)methanesulfonamide as an oil (5 mg).

¹H-NMR (DMSO-d₆) δ: 1.23-1.43 (5H, m), 1.89-2.19 (4H, m), 2.95 (3H, s), 3.31-3.37 (1H, m), 6.77 (1H, d, J=10.0 Hz), 7.06 (1H, d, J=7.5 Hz), 7.22 (1H, d, J=6.5 Hz), 7.8 (1H, d, J=9.5 Hz), 8.17 (1H, s), 8.22 (2H, d, J=6.0 Hz), 8.61 (2H, d, J=6.0 Hz).

MS: 387 (M+H)⁺.

### Example 114

### N-[1-(methylsulfonyl)-3-piperidinyl]-3-(4-pyridinyl) imidazo[1,2-b]pyridazin-6-amine

¹H-NMR (DMSO-d₆) δ: 1.55-2.18 (5H, m), 2.62-2.72 (1H, m), 2.80-3.00 (1H, m), 2.89 (3H, s), 3.32-3.50 (2H, m), 3.88 (1H, brs), 3.98-4.04 (1H, m), 6.83 (1H, d, J=9.6 Hz), 7.28 (1H, d, J=6.6 Hz), 7.85 (1H, d, J=9.6 Hz), 8.13-8.16 (3H, m), 8.57-8.60 (2H, m).

MS: 373 (M+H)⁺, 395 (M+Na)⁺.

### Example 115

To a solution of 6-chloro-3-(4-pyridinyl)imidazo[1,2-b]pyridazine (80 mg) and ethyl 4-amino-1-piperidinecarboxylate (179.2 mg) in toluene (6.9 mL) was added tris (dibenzylidenacetone) dipalladium chloroform complex (═Pd₂ dba₃-CHCl₃, 10.8 mg), 2,2′-Bis(diphenylphosphino)-1,1′-binaphthyl (=BINAP, 19.5 mg), and sodium tert-butoxide (150.0 mg), which was stirred at 110° C. for 2.5 hours under nitrogen atmosphere. The solvent was evaporated, the residue was dissolved in dimethylsulfoxide and the resulting solution was desalted using solid-phase extraction cartridge. Evaporation of the solvent gave a residue, which was purified by column chromatography on silica gel to give 4-{[3-(4-pyridinyl)imidazo[1,2-b]pyridazin-6-yl]amino}-1-piperidinecarboxylate (39.1 mg) as a brown powder.

¹H-NMR (DMSO-d₆) δ: 1.20 (3H, t, J=4.5 Hz), 1.42 (2H, ddd, J=3.5, 10.5, 20.6 Hz), 1.99-2.20 (2H, m), 3.11 (2H, dd, J=10.5, 10.5 Hz), 3.78-4.02 (3H, m), 4.06 (2H, q, J=7.0 Hz), 6.78 (1H, d, J=9.7 Hz), 7.24 (1H, d, J=6.7 Hz), 7.83 (1H, d, J=9.6 Hz), 8.17 (1H, s), 8.19 (2H, d, J=5.0 Hz), 8.62 (2H, d, J=6.5 Hz).

MS: 367 (M+H)⁺.

The following compounds were obtained in a similar manner to that of Example 115.

### Example 116

### N-[(3R)-1-Benzyl-3-pyrrolidinyl]-3-(4-pyridinyl)imidazo[1,2-b]pyridazin-6-amine

¹H-NMR (DMSO-d₆) δ: 1.64-1.86 (1H, m), 2.21-2.80 (4H, m), 2.81-3.05 (1H, m), 3.59 (1H, d, J=13.0 Hz), 3.68 (1H, d, J=13.0 Hz), 4.17-4.37 (1H, m), 6.79 (1H, d, J=9.5 Hz), 7.14-7.49 (6H, m), 7.81 (1H, d, J=10.0 Hz), 8.15 (1H, s), 8.17 (2H, d, J=6.0 Hz), 8.59 (2H, d, J=6.0 Hz).

MS: 371 (M+H)⁺.

### Example 117

### N-Cyclopentyl-3-(4-pyridinyl)imidazo[1,2-b]pyridazin-6-amine

¹H-NMR (DMSO-d₆) δ: 1.47-1.83 (6H, m), 1.91-2.15 (2H, m), 4.00-4.20 (1H, m), 6.76 (1H, d, J=9.7 Hz), 7.21 (1H, d, J=5.8 Hz), 7.79 (1H, d, J=9.8 Hz), 8.16 (1H, s), 8.23 (2H, d, J=6.0 Hz), 8.61 (2H, d, J=6.0 Hz).

MS: 280 (M+H)⁺.

### Example 118

### N-[(3S)-1-Benzyl-3-pyrrolidinyl]-3-(4-pyridinyl)imidazo[1,2b]pyridazin-6-amine

¹H-NMR (DMSO-d₆) δ: 1.64-1.86 (1H, m), 2.21-2.80 (4H, m), 2.81-3.05 (1H, m), 3.59 (1H, d, J=13.0 Hz), 3.68 (1H, d, J=13.0 Hz), 4.17-4.37 (1H, m), 6.79 (1H, d, J=9.5 Hz), 7.14-7.49 (6H, m), 7.81 (1H, d, J=10.0 Hz), 8.15 (1H, s), 8.17 (2H, d, J=6.0 Hz), 8.59 (2H, d, J=6.0 Hz).

MS: 371 (M+H)⁺.

### Example 119

### N-(1-Benzyl-4-piperidinyl)-3-(4-pyridinyl)imidazo[1,2-b]pyridazin-6-amine

¹H-NMR (DMSO-d₆) δ: 1.52 (2H, dd, J=10.0, 20.6 Hz), 2.00-2.28 (4H, m), 2.68-2.97 (2H, m), 3.52 (2H, s), 3.56-3.78 (1H, m), 6.78 (1H, d, J=9.8H z), 7.15-7.40 (6H, m), 7.81 (1H, d, J=9.8 Hz), 8.16 (1H, s), 8.18 (2H, dd, J=1.5, 5.0 Hz), 8.60 (2H, dd, J=1.0, 5.0 Hz).

MS: 385 (M+H)⁺.

### Example 120

### tert-butyl (3R)-3-{[3-(4-Pyridinyl)imidazo[1,2-b]pyridazin-6-yl]amino}-1-pyrrolidinecarboxylate

¹H-NMR (DMSO-d₆) δ: 1.23-1.50 (10H, m), 1.89-2.11 (1H, m), 2.12-2.35 (1H, m), 3.27-3.51 (2H, m), 3.59-3.81 (1H, m), 4.22-4.41 (1H, m), 6.79 (1H, d, J=9.7 Hz), 7.48 (1H, d, J=5.1 Hz), 7.85 (1H, d, J=9.7 Hz), 8.19 (1H, s), 8.19 (2H, d, J=6.0 Hz), 8.61 (2H, d, J=6.0 Hz).

MS: 381 (M+H)⁺, 403 (M+Na)⁺.

### Example 121

### tert-Butyl 4-{[3-(4-pyridinyl)imidazo[1,2-b]pyridazin-6-yl]amino}-1-piperidinecarboxylate

¹H-NMR (DMSO-d₆) δ: 1.20-1.56 (2H, m), 1.42 (9H, s), 2.00-2.15 (2H, m), 2.90-3.20 (2H, m), 3.73-4.14 (3H, m), 6.77 (1H, d, J=9.7 Hz), 7.22 (1H, d, J=6.6 Hz), 7.82 (1H, d, J=9.7 Hz), 8.17 (1H, s), 8.18 (2H, d, J=5.5 Hz), 8.62 (2H, d, J=6.0 Hz).

MS: 395 (M+H)⁺, 417 (M+Na)⁺.

### Example 122

To a stirred mixture of N-[4-(benzyloxy)phenyl]-3-(4-pyridinyl)imidazo[1,2-b]pyridazin-6-amine (80 mg) and cyclohexene (648 mg) in ethanol/tetrahydrofuran (1 mL/1 mL) was added palladium hydroxide (═Pd(OH)₂, 16 mg) at ambient temperature. The resulting mixture was stirred at 80° C. for 5 hours under nitrogen. The mixture was filtered through Celite and washed with ethanol and tetrahydrofuran, successively. The filtrate was concentrated in vacuo. The residue was purified by silica gel column chromatography eluting with chloroform/methanol (50:1 to 20:1) to give 4-{[3-(4-pyridinyl)imidazo[1,2-b]pyridazin-6-yl]amino}phenol (26.3 mg).

¹H-NMR (DMSO-d₆) δ: 6.81 (2H, d, J=9.0 Hz), 6.95 (1H, d, J=9.5 Hz), 7.48 (2H, d, J=9.0 Hz), 7.93 (1H, d, J=9.5 Hz), 8.13 (2H, d, J=6.0 Hz), 8.18 (1H, s), 8.61 (2H, d, J=6.0 Hz), 9.23 (1H, bs), 9.24 (1H, bs).

MS: 304 (M+H)⁺.

The following compounds were obtained in a similar manner to that of Example 122.

### Example 123

### 3-{[3-(4-Pyridinyl)imidazo[1,2-b]pyridazin-6-yl]amino}phenol

¹H-NMR (DMSO-d₆) δ: 6.45-6.54 (1H, m), 7.02 (1H, d, J=9.5 Hz), 7.11-7.26 (3H, m), 7.99 (1H, d, J=9.5 Hz), 8.18 (2H, d, J=6.5 Hz), 8.22 (1H, s), 8.64 (2H, d, J=6.5 Hz), 9.44 (1H, bs), 9.48 (1H, bs).

MS: 304 (M+H)⁺.

### Example 124

### 2-{[3-(4-Pyridinyl)imidazo[1,2-b]pyridazin-6-yl]amino}phenol

MS: 304 (M+H)⁺.

### Example 125

### 2-Chloro-4-{6-[(trans-4-hydroxycyclohexyl)amino]imidazo[1,2-b]pyridazin-3-yl}phenol

¹H-NMR (DMSO-d₆) δ: 1.21-1.42 (4H, m), 1.86-1.94 (2H, m), 2.10-2.17 (2H, m), 3.35-3.61 (2H, m), 4.60 (1H, brs), 6.64 (1H, d, J=9.6 Hz), 6.95 (1H, d, J=7.2 Hz), 7.05 (1H, d, J=8.4 Hz), 7.70 (1H, d, J=9.6 Hz), 7.81-7.83 (2H, m), 8.44 (1H, d, J=2.0 Hz), 10.36 (1H, brs).

MS: 359 (M+H)⁺.

### Example 126

### 2-Hydroxy-5-{6-[(trans-4-hydroxycyclohexyl)amino]imidazo[1,2-b]pyridazin-3-yl}benzonitrile

¹H-NMR (DMSO-d₆) δ: 1.22-1.40 (4H, m), 1.86-1.93 (2H, m), 2.09-2.16 (2H, m), 3.35-3.58 (2H, m), 4.61 (1H, brs), 6.66 (1H, d, J=9.6 Hz), 6.96 (1H, d, J=7.2 Hz), 7.06 (1H, d, J=8.8 Hz), 7.71 (1H, d, J=9.6 Hz), 7.82 (1H, brs), 8.16 (1H, m), 8.49 (1H, d, J=2.4 Hz).

MS: 350 (M+H)⁺.

### Example 127

### 2,6-dichloro-4-{6-[(trans-4-hydroxycyclohexyl)amino]imidazo[1,2-b]pyridazin-3-yl}phenol

¹H-NMR (DMSO-d₆) δ: 1.15-1.47 (4H, m), 1.85-2.17 (4H, m), 3.32-3.61 (2H, m), 4.61 (1H, brs), 6.67 (1H, d, J=9.6 Hz), 7.00 (1H, d, J=6.4 Hz), 7.72 (1H, d, J=9.6 Hz), 7.95 (1H, s), 8.29 (2H, s), 10.28 (1H, brs).

MS: 393 (M+H)⁺.

### Example 128

To a suspension of N-4-piperidinyl-3-(4-pyridinyl)imidazo[1,2-b]pyridazin-6-amine trihydrochloride (100 mg) in N,N-dimethylformamide (2.0 ml) was added K₂CO₃ (171.1 mg), 1,1′-(bromomethylene)dibenzene (67.3 mg), the mixture was stirred at 60° C. for 2 hours. The reaction mixture was poured into water and extracted with ethyl acetate. The organic extract was washed with water twice and brine, dried over magnesium sulfate and concentrated in vacuo. The residue was purified by silica gel column chromatography eluted with 5%-10% methanol in chloroform to give N-[1-(diphenylmethyl)-4-piperidinyl]-3-(4-pyridinyl)imidazo[1,2-b]pyridazin-6-amine (32 mg).

¹H-NMR (DMSO-d₆) δ: 1.60-1.70 (2H, m), 2.03-2.14 (4H, m), 2.78-2.84 (2H, m), 3.63 (1H, m), 4.38 (1H, s), 6.78 (1H, d, J=9.7 Hz), 7.15-7.48 (12H, m), 7.79 (1H, d, J=9.7 Hz), 8.14-8.17 (2H, m), 8.55-8.65 (2H, m).

MS: 461 (M+H)⁺.

The following compound was obtained in a similar manner to that of Example 128.

### Example 129

### N-(1-Benzyl-3-piperidinyl)-3-(4-pyridinyl)imidazo[1,2-b]pyridazin-6-amine

¹H-NMR (DMSO-d₆) δ: 1.22-1.36 (1H, m), 1.40-1.91 (2H, m), 1.95-2.14 (3H, m), 2.68-2.73 (1H, m), 3.06-3.11 (1H, m), 3.53 (2H, dd, J=13.4 Hz), 3.87 (1H, brs), 6.78 (1H, d, J=9.6 Hz), 7.08-7.35 (6H, m), 7.79 (1H, d, J=9.6 Hz), 8.15-8.18 (3H, m), 8.59-8.62 (2H, m).

MS: 385.

### Example 130

To a solution of N-4-piperidinyl-3-(4-pyridinyl)imidazo[1,2-b]pyridazin-6-amine trihydrochloride (56 mg) and triethylamine (81.2 μl), in N,N-dimethylformamide (1.12 ml) was added benzoyl chloride (16.9 μl), the mixture was stirred at 23° C. for 1 hour. The reaction mixture was poured into water and extracted with ethyl acetate. The organic extract was washed with water twice and brine, dried over magnesium sulfate and concentrated in vacuo. The residue was purified by silica gel column chromatography eluted with chloroform/methanol (95:5 to 90:10) to give N-(1-benzoyl-4-piperidinyl)-3-(4-pyridinyl)imidazo[1,2-b]pyridazin-6-amine (41 mg).

¹H-NMR (DMSO-d₆) δ: 1.42-1.60 (2H, brs), 2.13 (2H, brs), 3.34 (4H, brs), 3.53-4.36 (1H, m), 3.96 (1H, brs), 6.79 (1H, d, J=9.7 Hz), 7.28 (1H, d, J=6.6 Hz), 7.39-7.49 (5H, m), 7.83 (1H, d, J=9.7 Hz), 8.17 (1H, s), 8.19-8.20 (2H, m), 8.62 (2H, dd, J=1.4, 4.9 Hz).

MS: 399 (M+H)⁺.

### Example 131

To a solution of N-(trans-4-methoxycyclohexyl)-3-(4-pyridinyl)imidazo[1,2-b]pyridazin-6-amine (50 mg) was added m-chloroperoxybenzoic acid (33.4 mg) and the mixture was stirred at ambient temperature for 2 hours. The reaction mixture was washed with a mixture of saturated NaHCO₃ aqueous solution and brine, 5% sodium sulfite aqueous solution, water and brine, successively. The organic phase was dried over magnesium sulfate, and concentrated in vacuo. The residue was purified by silica gel column chromatography eluting with chloroform/methanol (40:1 to 20:1) to give N-(trans-4-methoxycyclohexyl)-3-(1-oxido-4-pyridinyl)imidazo[1,2-b]pyridazin-6-amine (34.5 mg).

¹H-NMR (DMSO-d₆) δ: 1.20-1.43 (4H, m), 1.98-2.19 (4H, m), 3.14-3.30 (1H, m), 3.33 (3H, s), 3.55-3.78 (1H, m), 6.74 (1H, d, J=9.8 Hz), 7.13 (1H, d, J=6.7 Hz), 7.78 (1H, d, J=9.8 Hz), 8.10 (1H, s), 8.20-8.32 (4H, m).

MS: 340 (M+H)⁺.

### Example 132

trans-4-[(3-Iodoimidazo[1,2-b]pyridazin-6-yl)amino]cyclohexanol (100 mg) and vinylbenzene (291 mg) were dissolved in N,N-dimethylformamide (2.5 ml). To this solution were added triethylamine (0.40 mL), water (0.40 mL) and dichlorobis (triphenylphosphine)palladium(II) (═PdCl₂(PPh₃)₂, 39.2 mg) at ambient temperature. The resulting mixture was subjected to microwave irradiation at 100° C. for 1 hour. After all starting material had been consumed, as judged by TLC plate, the reaction mixture was cooled to ambient temperature and diluted with ethyl acetate/water (10 mL/10 mL). The resulting solution was extracted with ethyl acetate three times, the organic layers were combined, dried over magnesium sulfate, and concentrated in vacuo. The residue was purified by silica gel column chromatography eluting with chloroform/methanol (40:1 to 20:1) to give a mixture of trans-4-({3-[(E)-2-phenylvinyl]imidazo[1,2-b]pyridazin-6-yl}amino)cyclohexanol (35.8 mg).

¹H-NMR (DMSO-d₆) δ: 1.18-1.48 (4H, m), 1.88-2.01 (2H, m), 2.08-2.22 (2H, m), 3.41-3.77 (2H, m), 4.64 (1H, d, J=4.1 Hz), 6.67 (1H, d, J=9.6 Hz), 6.97 (1H, d, J=6.6 Hz), 7.20-7.35 (2H, m), 7.42 (2H, d, J=7.2 Hz), 7.54 (2H, d, J=7.2 Hz), 7.63 (1H, s), 7.65-7.88 (2H, m).) and trans-4-{[3-(1-phenylvinyl)imidazo[1,2-b]pyridazin-6-yl]amino}cyclohexanol (5.6 mg).

¹H-NMR (DMSO-d₆) δ: 1.03-1.42 (4H, m), 1.88-2.10 (4H, m), 3.35-3.43 (1H, m), 3.55-3.70 (1H, m), 5.32 (1H, s), 5.56 (1H, d, J=1.6 Hz), 6.25 (1H, d, J=1.6 Hz), 6.48 (1H, d, J=9.6 Hz), 7.32-7.41 (7H, m), 7.58 (1H, d, J=9.6 Hz).

The following compounds were obtained in a similar manner to that of Example 132.

### Example 133

### trans-4-({3-[(E)-2-(4-Pyridinyl)vinyl]imidazo[1,2-b]pyridazin-6-yl}amino)cyclohexanol

MS: 336 (M+H)⁺.

### Example 134

### trans-4-({3-[(E)-2-(3-Pyridinyl)vinyl]imidazo[1,2-b]pyridazin-6-yl}amino)cyclohexanol

MS: 336 (M+H)⁺.

### Example 135

### trans-4-({3-[(E)-2-(2-Pyridinyl)vinyl]imidazo[1,2-b]pyridazin-6-yl}amino)cyclohexanol

MS: 336 (M+H)⁺.

### Example 136

trans-4-[(3-Iodoimidazo[1,2-b]pyridazin-6-yl)amino]cyclohexanol (100 mg), dichlorobis(triphenylphosphine) palladium(II) (PdCl₂(PPh₃)₂, 9.80 mg) and CuI (5.32 mg) were dissolved in N,N-dimethylformamide (2.5 mL) and triethylamine (2.5 ml). The mixture was stirred at ambient temperature for 10 minutes. Then ethynylbenzene (34.22 mg) was added. The resulting mixture was subjected to microwave irradiation at 80° C. for 1 hour. After all starting material had been consumed, as judged by TLC plate, the reaction mixture was cooled to ambient temperature and diluted with ethyl acetate/water (10 mL:10 mL). The resulting solution was extracted with ethyl acetate three times, the organic layers were combined, dried over magnesium sulfate, and concentrated in vacuo. The residue was purified by silica gel column chromatography eluting with chloroform/methanol (40:1 to 20:1) to give trans-4-{[3-(phenylethynyl)imidazo[1,2-b]pyridazin-6-yl]amino}cyclohexanol (68.6 mg).

¹H-NMR (DMSO-d₆) δ: 1.15-1.41 (4H, m), 1.80-1.99 (2H, m), 2.02-2.24 (2H, m), 3.40-3.70 (2H, m), 4.59 (1H, d, J=4.5 Hz), 6.72 (1H, d, J=9.6 Hz), 7.00 (1H, d, J=6.6 Hz) 7.40-7.59 (5H, m), 7.72 (1H, d, J=3.9 Hz), 7.75 (1H, d, J=5.7 Hz).

### Example 137

To a stirred mixture of trans-4-({3-[4-(benzyloxy)-3-fluorophenyl]imidazo[1,2-b]pyridazin-6-yl}amino) cyclohexanol (150 mg) and cyclohexene (1.5 mL) in ethanol/tetrahydrofuran (3 mL/3 mL) was added palladium hydroxide (═Pd(OH)₂, 60 mg) at ambient temperature. The resulting mixture was stirred at 80° C. for 5 hours under nitrogen. The mixture was filtered through Celite and washed with methanol and tetrahydrofuran, successively. The filtrate was concentrated in vacuo. The residue was purified by silica gel column chromatography eluting with chloroform/methanol (50:1 to 10:1) to give 2-fluoro-4-{6-[(trans-4-hydroxycyclohexyl)amino]imidazo[1,2-b]pyridazin-3-yl}phenol (35.6 mg).

¹H-NMR (DMSO-d₆) δ: 1.13-1.42 (4H, m), 1.82-2.00 (2H, m), 2.03-2.19 (2H, m), 3.34-3.62 (2H, m), 4.62 (1H, d, J=4.3 Hz), 6.64 (1H, d, J=9.6 Hz), 6.96 (1H, d, J=5.4 Hz), 7.04 (1H, d, J=9.2 Hz), 7.71 (1H, d, J=9.6 Hz), 7.75 (1H, d, J=9.2 Hz), 7.81 (1H, s), 8.17 (1H, dd, J=2.0, 13.7 Hz), 10.0 (1H, bs).

The following compound was obtained in a similar manner to that of Example 137.

### Example 138

### 5-{6-[(trans-4-Hydroxycyclohexyl)amino]imidazo[1,2-b]pyridazin-3-yl}-2-methoxyphenol

¹H-NMR (DMSO-d₆) δ: 1.20-1.32 (4H, m), 1.84-1.93 (2H, m), 2.05-2.13 (2H, m), 3.42-3.51 (1H, m), 3.56-3.69 (1H, m), 3.87 (3H, s), 4.60 (1H, d, J=4.4 Hz), 6.61 (1H, d, J=9.7 Hz), 6.84 (2H, d, J=8.2 Hz), 7.54 (1H, dd, J=8.2, 1.8 Hz), 7.68 (1H, d, J=9.7 Hz), 7.73 (1H, s), 7.77 (1H, d, J=1.8 Hz), 9.18 (1H, s).

MS: 355 (M+H)⁺.

### Example 139

To a stirred mixture of 3-(4-pyridinyl)imidazo[1,2-b]pyridazin-6-amine (30.0 mg) and acetic anhydride (43.5 mg) in pyridine (1.15 mL) was added 4-dimethylaminopyridine (3.47 mg) at ice-bath temperature. The reaction was stirred at 100° C. for 3 hours. After all starting material had been consumed, as judged by TLC plate, the reaction mixture was poured into 1M HCl aqueous solution (10 mL). The resulting solution was extracted with ethyl acetate three times, the organic layers were combined, dried over magnesium sulfate, and concentrated in vacuo. The residue was purified by silica gel column chromatography eluting with chloroform/methanol (40:1 to 20:1) to give N-[3-(4-pyridinyl)imidazo[1,2-b]pyridazin-6-yl]acetamide (12.6 mg).

¹H-NMR (DMSO-d₆) δ: 3.16 (3H, s), 7.95 (2H, d, J=2.1 Hz), 8.04 (1H, d, J=4.9 Hz), 8.14 (1H, s), 8.34 (1H, d, J=4.9 Hz), 8.73 (2H, d, J=2.1 Hz), 9.69 (1H, bs).

MS: 254 (M+H)⁺.

### Example 140

Diethylaminosulfur trifluoride (=DAST, 31 μl) was added dropwise to a solution of (trans-4-{[3-(4-pyridinyl) imidazo[1,2-b]pyridazin-6-yl]amino}cyclohexyl)methanol (23 mg) in dichloromethane (1 ml). After stirring at 0° C. for 4 hours, the reaction mixture was poured into saturated aqueous NaHCO₃, and extracted with dichloromethane 10% methanol. The organic layer was dried over magnesium sulfate, and evaporated in vacuo. The residue was purified by preparative TLC eluting with dichloromethane/methanol (10:1) to give N-[trans-4-(fluoromethyl)cyclohexyl]-3-(4-pyridinyl)imidazo[1,2-b]pyridazin-6-amine (1 mg).

¹H-NMR (DMSO-d₆) δ: 1.17-1.31 (5H, m), 1.77-1.88 (2H, m), 2.15-2.29 (2H, m), 3.50-3.70 (1H, m), 4.21 (1H, d, J=5.9 Hz), 4.45 (1H, d, J=5.9H), 6.76 (1H, d, J=9.7 Hz), 7.16 (1H, d, J=6.1 Hz), 7.80 (1H, d, J=9.7 Hz), 8.15 (1H, s), 8.20 (2H, d, J=6.3 Hz), 8.60 (2H, d, J=6.3 Hz).

MS: 326 (M+H)⁺.

The following compounds were obtained in a similar manner to that of Example 140.

### Example 141

### N-[cis-4-(Fluoromethyl)cyclohexyl]-3-(4-pyridinyl)imidazo[1,2-b]pyridazin-6-amine

¹H-NMR (DMSO-d₆): 1.40-2.00 (9H, m), 3.93-4.05 (1H, m), 4.22 (0.5H, d, J=5.2 Hz), 4.46 (0.5H, d, J=6.0 Hz), 5.76 (0.5H, s), 6.55 (0.5H, s), 6.89 (1H, d, J=9.5 Hz), 7.07 (1H, d, J=5.8 Hz), 7.8 (1H, d, J=9.6 Hz), 8.16 (1H, s), 8.19 (2H, d, J=5.3 Hz), 8.6 (2H, d, J=5.3 Hz).

MS: 326 (M+H)⁺.

### Example 142

### N-(3-Fluoro-1-adamantyl)-3-(4-pyridinyl)imidazo[1,2-b]pyridazin-6-amine

¹H-NMR (DMSO-d₆): 1.57-1.63 (2H, m), 1.85-1.93 (4H, m), 2.04-2.18 (4H, m), 2.27-2.42 (4H, m), 6.80 (1H, d, J=10.0 Hz), 7.01 (1H, s), 7.80 (1H, d, J=10.0 Hz), 8.11-8.17 (3H, m), 8.61 (2H, d, J=6.0 Hz).

MS: 364 (M+H)⁺.

### Example 143

### N-(5-fluoroadamantan-2-yl)-3-(4-pyridinyl)imidazo[1,2-b]pyridazin-6-amine

¹H-NMR (DMSO-d₆) δ: 1.60-1.95 (8H, m), 2.12-2.36 (3H, m), 2.46-2.59 (2H, m), 3.80-3.94 (1H, m), 6.94 (1H, d), 7.19 (1H, d), 7.83 (1H, d), 8.17 (1H, s), 8.20 (2H, d), 8.59 (2H, d).

MS: 364 (M+H)⁺.

### Example 144

### N-(5-fluoroadamantan-2-yl)-3-(4-pyridinyl)imidazo[1,2-b]pyridazin-6-amine

¹H-NMR (DMSO-d₆) δ: 1.62-1.96 (8H, m), 2.13-2.37 (3H, m), 2.48-2.58 (2H, m), 3.78-3.93 (1H, m), 6.94 (1H, d), 7.19 (1H, d), 7.83 (1H, d), 8.17 (1H, s), 8.20 (2H, d), 8.59 (2H, d).

MS: 364 (M+H)⁺.

### Example 145

To a mixture of trans-4-[(3-iodoimidazo[1,2-b]pyridazin-6-yl)amino]cyclohexanol (80 mg) and 2-(N,N-dimethylamino) pyridine-5-boronic acid hydrate (74.1 mg) in 1,2-dimethoxyethane (400 uL) was added tetrakis (triphenylphosphine)palladium(0) (25.8 mg), sodium hydroxide (35.7 mg), and water (200 μL), which was subjected to microwave irradiation at 135° C. for 30 minutes. To the resultant was added water. The mixture was extracted with dichloromethane. The organic layer was washed with brine, dried over sodium sulfate, filtered, and evaporated in vacuo. The residue was purified by column chromatography on silica gel to give trans-4-({3-[6-(dimethylamino)-3-pyridinyl]imidazo[1,2-b]pyridazin-6-yl}amino)cyclohexanol (49.7 mg) as a yellow powder.

MS (ES+): 353 (M+H)⁺.

The following compounds were obtained in a similar manner to that of Example 145.

### Example 146

### trans-4-{[3-(1,3-Benzodioxol-5-yl)imidazo[1,2-b]pyridazin-6-yl]amino}cyclohexanol

MS: 353 (M+H)⁺.

### Example 147

### trans-4-{[3-(2-Methoxy-5-pyrimidinyl)imidazo[1,2-b]pyridazin-6-yl]amino}cyclohexanol

MS: 341 (M+H)⁺.

### Example 148

### trans-4-{[3-(6-Methoxy-3-pyridinyl)imidazo[1,2-b]pyridazin-6-yl]amino}cyclohexanol

MS: 340 (M+H)⁺.

### Example 149

### trans-4-{[3-(3-Chloro-4-fluorophenyl)imidazo[1,2-b]pyridazin-6-yl]amino}cyclohexanol

¹H-NMR (DMSO-d₆) δ: 1.22-1.38 (4H, m), 1.86-1.93 (2 Hm), 2.11-2.15 (2H, m), 3.35-3.55 (2H, m), 4.61 (1H, brs), 6.70 (1H, d, J=9.8 Hz), 7.04 (1H, d, J=6.7 Hz), 7.76 (1H, d, J=9.8 Hz), 7.71 (1H, d, J=9.6 Hz), 7.98 (1H, brs), 8.06 (1H, m), 8.68 (1H, m).

MS: 361 (M+H)⁺.

### Example 150

### trans-4-{[3-(4-Chloro-3-fluorophenyl)imidazo[1,2-b]pyridazin-6-yl]amino}cyclohexanol

MS: 361 (M+H)⁺.

### Example 151

### trans-4-{[3-(1-Methyl-1H-pyrazol-4-yl)imidazo[1,2-b]pyridazin-6-yl]amino}cyclohexanol

¹H-NMR (DMSO-d₆) δ: 1.24-1.47 (4H, m), 1.89-1.94 (2H, m), 2.14-2.17 (2H, m), 3.48-3.61 (2H, brs), 3.92 (3H, s), 4.61 (1H, d, J=4.4 Hz), 6.55-6.62 (1H, m), 6.89 (1H, d, J=6.6 Hz), 6.61-6.96 (2H, m), 8.08 (1H, s), 8.29 (1H, s).

MS: 313 (M+H)⁺, 335 (M+Na)⁺.

### Example 152

### trans-4-{[3-(2,6-Dichloro-4-pyridinyl)imidazo[1,2-b]pyridazin-6-yl]amino}cyclohexanol

¹H-NMR (DMSO-d₆) δ: 1.25-1.91 (4H, m), 1.91-1.99 (2H, m), 2.11-2.23 (2H, m), 3.54 (2H, brs), 4.63 (1H, brs), 3.79 (1H, d, J=9.6 Hz), 7.23 (1H, d, J=6.6 Hz), 7.81 (1H, d, J=9.6 Hz), 8.37 (1H, s), 8.42 (1H, s).

MS: 401 (M+Na)⁺.

### Example 153

### trans-4-{[3-(2-Fluoro-4-pyridinyl)imidazo[1,2-b]pyridazin-6-yl]amino}cyclohexanol

¹H-NMR (DMSO-d₆) δ: 1.25-1.39 (4H, m), 1.90 (2H, brs), 2.17 (2H, m), 3.51 (2H, brs), 4.64 (1H, d, J=4.5 Hz), 6.79 (1H, d, J=9.6 Hz), 7.20 (1H, d, J=6.6 Hz), 7.82 (1H, d, J=9.6 Hz), 8.04 (1H, d, J=5.5 Hz), 8.17 (1H, s), 8.25 (1H, d, J=5.5 Hz), 8.27 (1H, s).

MS: 328 (M+H)⁺, 350 (M+Na)⁺.

### Example 154

### trans-4-{[3-(2-Bromo-4-pyridinyl)imidazo[1,2-b]pyridazin-6-yl]amino}cyclohexanol

¹H-NMR (DMSO-d₆) δ: 1.25-1.46 (4H, m), 1.90-1.92 (2H, m), 2.12-2.15 (2H, m), 3.46-3.50 (2H, m), 3.57-3.61 (1H, m), 6.82 (1H, d, J=9.6 Hz), 7.24 (1H, d, J=6.6 Hz), 7.83 (1H, d, J=9.6 Hz), 8.09 (1H, dd, J=1.4, 5.3 Hz), 8.31 (1H, s), 8.39 (1H, d, J=5.3 Hz), 8.71 (1H, d, J=1.4 Hz).

MS: 389 (M+H)⁺.

### Example 155

### 6-Chloro-3-(2-chloro-4-pyridinyl)-8-methylimidazo[1,2-b]pyridazine

¹H-NMR (DMSO-d₆) δ: 2.46 (3H, s), 8.17 (1H, dd), 8.26 (1H, d), 8.33 (1H, d), 8.52 (1H, d), 8.63 (1H, s).

MS: 301 (M)⁺, 303 (M+2)⁺.

### Example 156

### 6-[(E)-2-Phenylvinyl]-3-(4-pyridinyl)imidazo[1,2-b]pyridazine

¹H-NMR (DMSO-d₆) δ: 7.30-7.53 (4H, m), 7.72-7.92 (4H, m), 8.20-8.35 (3H, m), 8.54 (1H, s), 8.71 (2H, d).

MS: 299 (M+H)⁺.

### Example 157

### 6-Chloro-8-methoxy-3-(4-pyridinyl)imidazo[1,2-b]pyridazine

MS: 261 (M+H)⁺.

### Example 158

### trans-4-({3-[2-(4-Methyl-1-piperazinyl)-4-pyridinyl]imidazo[1,2-b]pyridazin-6-yl}amino)cyclohexanol

MS: 408 (M+H)⁺.

### Example 159

### 2-(Benzyloxy)-5-{6-[(trans-4-hydroxycyclohexyl)amino]imidazo[1,2-b]pyridazin-3-yl}benzonitrile

MS: 462 (M+Na)⁺.

### Example 160

### trans-4-({3-[4-(Benzyloxy)-3-chlorophenyl]imidazo[1,2-b]pyridazin-6-yl}amino)cyclohexanol

MS: 449 (M+H)⁺.

### Example 161

### 1-(3-{6-[(trans-4-Hydroxycyclohexyl)amino]imidazo[1,2-h]pyridazin-3-yl}phenyl)ethanone

¹H-NMR (DMSO-d₆) δ: 1.14-1.43 (4H, m), 1.80-1.94 (2H, m), 2.03-2.15 (2H, m), 2.67 (3H, s), 3.36-3.76 (2H, m), 4.58 (1H, d, 4.0 Hz), 6.70 (1H, d, J=9.6 Hz), 6.95 (1H, d, J=7.6 Hz), 7.60 (1H, t, J=7.6 Hz), 7.75 (1H, d, J=9.6 Hz), 7.87-7.90 (1H, m), 7.97 (1H, s), 8.32-8.39 (1H, m), 8.80-8.83 (1H, m).

MS: 351 (M+H)⁺.

### Example 162

### 4-{6-[(trans-4-Hydroxycyclohexyl)amino]imidazo[1,2-h]pyridazin-3-yl}-2,6-dimethylphenol

MS: 353 (M+H)⁺.

### Example 163

### trans-4-({3-[4-(Benzyloxy)-3,5-dichlorophenyl]imidazo[1,2-b]pyridazin-6-yl}amino)cyclohexanol

¹H-NMR (DMSO-d₆) δ: 1.24-1.44 (4H, m), 1.88-1.94 (2H, m), 2.12-2.17 (2H, m), 3.43-3.61 (2H, m), 4.61 (1H, d, J=4.2 Hz), 5.08 (2H, s), 6.71 (1H, d, J=9.6 Hz), 7.08 (1H, d, J=6.8 Hz), 7.39-7.47 (3H, m), 7.56-7.59 (2H, m), 7.76 (1H, d, J=9.6 Hz), 8.09 (1H, s), 8.46 (2H, s).

MS: 483 (M+H)⁺.

### Example 164

### N-(3-{6-[(trans-4-Hydroxycyclohexyl)amino]imidazo[1,2-b]pyridazin-3-yl}phenyl)acetamide

MS: 388 (M+Na)⁺.

### Example 165

### N-(4-{6-[(trans-4-Hydroxycyclohexyl)amino]imidazo[1,2-b]pyridazin-3-yl}-2-methoxyphenyl)-1-methyl-1H-indole-2-carboxamide

¹H-NMR (DMSO-d₆) δ: 1.23-1.34 (4H, m), 1.86-1.93 (2H, m), 2.07-2.15 (2H, m), 3.42-3.51 (1H, m), 3.63-3.71 (1H, m), 3.99 (3H, s), 4.04 (3H, s), 4.61 (1H, d, J=4.4 Hz), 6.68 (1H, d, J=9.6 Hz), 6.94 (1H, d, J=7.2 Hz), 7.13-7.17 (1H, m), 7.31-7.35 (2H, m), 7.57-7.59 (1H, m), 7.69-7.71 (1H, m), 7.74 (1H, d, J=9.6 Hz), 7.81-7.84 (1H, m), 7.93-7.97 (3H, m), 9.43 (1H, s).

MS: 511 (M+H)⁺.

### Example 166

AcOH (300 μl) was added to a suspension of trans-4-([3-(4-pyridinyl)imidazo[1,2-b]pyridazin-6-yl]amino)cyclohexanol (50 mg) in dichloromethane (3.3 ml). Then, methylisocyanate (30 μl) was added to the mixture. After stirring at ambient temperature for overnight, the reaction mixture was evaporated in vacuo. The residue was purified by preparative TLC eluting with dichloromethane/methanol (10:1) to give trans-4-{[3-(4-pyridinyl)imidazo[1,2-b]pyridazin-6-yl]amino}cyclohexyl methylcarbamate (27 mg).

¹H-NMR (DMSO-d₆) δ: 1.25-1.63 (4H, m), 1.95-2.26 (4H, m), 2.57 (3H, d, J=4.5 Hz), 3.57-3.76 (1H, m), 4.49-4.67 (1H, m), 6.77 (1H, d, J=10.0 Hz), 6.95 (1H, q, J=4.5 Hz), 7.16 (1H, d, J=6.5 Hz), 7.8 (1H, d, J=9.5 Hz), 8.16 (1H, s), 8.2 (2H, d, J=6.5 Hz), 8.6 (2H, d, J=6.5 Hz).

MS: 367 (M+H)⁺.

The following compounds were obtained in a similar manner to that of Example 166.

### Example 167

### trans-4-{[3-(4-Pyridinyl)imidazo[1,2-b]pyridazin-6-yl]amino}cyclohexyl ethylcarbamate

MS: 381 (M+H)⁺.

### Example 168

### N-Methyl-N′-(trans-4-{[3-(4-pyridinyl)imidazo[1,2-b]pyridazin-6-yl]amino}cyclohexyl)urea

¹H-NMR (DMSO-d₆) δ: 1.24-1.38 (4H, m), 1.86-1.96 (2H, m), 2.10-2.23 (2H, m), 2.54 (3H, d, J=5.0 Hz), 3.52-3.71 (1H, m), 5.57-5.76 (2H, m), 5.82 (1H, d, J=8.0 Hz), 6.76 (1H, d, J=9.6 Hz), 7.15 (1H, d, J=6.8 Hz), 7.80 (1H, d, J=9.8 Hz), 8.16 (1H, s), 8.21 (2H, d, J=6.2 Hz), 8.60 (2H, d, J=6.2 Hz).

MS: 366 (M+H)⁺.

### Example 169

To a stirred mixture of trans-4-[(3-iodoimidazo[1,2-b]pyridazin-6-yl)amino]cyclohexanol (100 mg) and (4-methylphenyl)boronic acid (76.0 mg) in dioxane (3 ml) was added 2M sodium carbonate aqueous solution (0.447 mL) at ambient temperature. The suspension turned to a clear yellow solution. palladium acetate (II) (═Pd(OAc)₂, 3.13 mg) and triphenylphosphine (14.6 mg) were then added to the mixture at ambient temperature. After addition, the resulting mixture was subjected to microwave irradiation at 100° C. for 1 hour. The reaction mixture was cooled to ambient temperature and diluted with ethyl acetate/water (20 mL/20 mL). The resulting solution was extracted with ethyl acetate three times. The organic layers were combined, dried over magnesium sulfate, and concentrated in vacuo. The residue was purified by silica gel column chromatography eluting with chloroform/methanol (50:1 to 20:1) to give trans-4-{[3-(4-methylphenyl)imidazo[1,2-b]pyridazin-6-yl]amino}cyclohexanol (63.7 mg).

¹H-NMR (DMSO-d₆) δ: 1.15-1.42 (4H, m), 1.82-2.00 (2H, m), 2.01-2.21 (2H, m), 2.35 (3H, s), 3.35-3.64 (2H, m), 4.61 (1H, d, J=4.3 Hz), 6.64 (1H, d, J=9.6 Hz), 6.93 (1H, d, J=6.6 Hz), 7.26 (2H, d, J=8.2 Hz), 7.72 (1H, d, J=9.6 Hz), 7.83 (1H, s), 8.10 (2H, d, J=8.2 Hz).

MS: 323 (M+H)⁺.

The following compounds were obtained in a similar manner to that of Example 169.

### Example 170

### 3-{6-[(trans-4-Hydroxycyclohexyl)amino]imidazo[1,2-b]pyridazin-3-yl}benzonitrile

¹H-NMR (DMSO-d₆) δ: 1.16-1.48 (4H, m), 1.78-1.99 (2H, m) 2.02-2.28 (2H, m), 3.40-3.65 (2H, m), 4.60 (1H, d, J=4.0 Hz), 6.73 (1H, d, J=9.8 Hz), 7.05 (1H, d, J=6.7 Hz), 7.65 (1H, dd, J=6.2, 8.0 Hz), 7.77 (1H, d, J=6.2 Hz), 7.78 (1H, d, J=9.8 Hz), 8.05 (1H, s), 8.45 (1H, d, J=8.0 Hz), 8.75 (1H, s).

MS: 334 (M+H)⁺.

### Example 171

### trans-4-{[3-(4-Fluorophenyl)imidazo[1,2-b]pyridazin-6-yl]amino}cyclohexanol

¹H-NMR (DMSO-d₆) δ: 1.18-1.43 (4H, m), 1.79-2.00 (2H, m), 2.01-2.21 (2H, m), 3.39-3.63 (2H, m), 4.59 (1H, d, J=4.4 Hz), 6.67 (1H, d, J=9.6 Hz), 6.96 (1H, d, J=6.8 Hz), 7.29 (2H, dd, J=9.0, 9.0 Hz), 7.73 (1H, d, J=9.6 Hz), 7.86 (1H, s), 8.23 (2H, dd, J=5.5, 9.0 Hz).

MS: 338 (M+H)⁺.

### Example 172

### trans-4-({3-[4-(Dimethylamino)phenyl]imidazo[1,2-b]pyridazin-6-yl}amino)cyclohexanol

MS: 352 (M+H)⁺.

### Example 173

### trans-4-{[3-(4-Biphenylyl)imidazo[1,2-b]pyridazin-6-yl]amino}cyclohexanol

MS: 385 (M+H)⁺.

### Example 174

### trans-4-{[3-(3-Fluorophenyl)imidazo[1,2-b]pyridazin-6-yl]amino}cyclohexanol

¹H-NMR (DMSO-d₆) δ: 1.15-1.46 (4H, m), 1.80-2.01 (2H, m), 2.02-2.28 (2H, m), 3.39-3.62 (2H, m), 4.61 (1H, d, J=4.5 Hz), 6.70 (1H, d, J=9.6 Hz), 7.04 (1H, d, J=6.5 Hz), 7.13 (1H, dt, J=2.6, 8.3 Hz), 7.48 (1H, dd, J=8.1, 14.5 Hz), 7.76 (1H, d, J=9.6 Hz), 7.95 (1H, d, J=8.0 Hz), 8.00 (1H, s), 8.26 (1H, d, J=11.0 Hz).

MS: 327 (M+H)⁺.

### Example 175

### 3-{6-[(trans-4-Hydroxycyclohexyl)amino]imidazo[1,2-b]pyridazin-3-yl}benzamide

MS: 352 (M+H)⁺.

### Example 176

### Methyl 3-{6-[(trans-4-Hydroxycyclohexyl)amino]imidazo[1,2-b]pyridazin-3-yl}benzoate

¹H-NMR (DMSO-d₆) δ: 1.15-1.40 (4H, m), 1.84-1.90 (2H, m), 2.05-2.18 (2H, m), 3.39-3.55 (1H, m), 3.35-3.75 (1H, m), 3.91 (3H, s), 4.59 (1H, d, J=3.9 Hz), 6.71 (1H, d, J=9.6 Hz), 6.98 (1H, d, J=6.9 Hz), 7.60 (1H, dd, J=7.8, 7.8 Hz), 7.76 (1H, d, J=9.6 Hz), 7.90 (1H, d, J=7.8 Hz), 7.95 (1H, s), 8.31 (1H, d, J=7.8 Hz), 8.93 (1H, s).

MS: 367 (M+H)⁺.

### Example 177

### trans-4-{[3-(5-Methoxy-3-pyridinyl)imidazo[1,2-b]pyridazin-6-yl]amino}cyclohexanol

MS: 362 (M+Na)⁺.

### Example 178

### trans-4-{[3-(3,4-Dimethoxyphenyl)imidazo[1,2-b]pyridazin-6-yl]amino}cyclohexanol

MS: 369 (M+H)⁺.

### Example 179

### trans-4-{[3-(2-Chloro-4-pyridinyl)imidazo[1,2-b]pyridazin-6-yl]amino}cyclohexanol

¹H-NMR (DMSO-d₆) δ: 1.02-1.50 (4H, m), 1.83-2.18 (4H, m), 3.39-3.60 (2H, m), 4.63 (1H, d, J=4.0 Hz), 6.77 (1H, d, J=9.7 Hz), 7.18 (1H, d, J=6.6 Hz), 7.80 (1H, d, J=9.7 Hz), 8.05 (1H, dd, J=1.4, 5.5 Hz), 8.27 (1H, s), 8.40 (1H, d, J=5.5 Hz), 8.57 (1H, d, J=1.4 Hz).

MS: 342 (M−H)⁻.

### Example 180

### 5-{6-[(trans-4-Hydroxycyclohexyl)amino]imidazo[1,2-b]pyridazin-3-yl}-2-pyridinecarbonitrile

MS: 333 (M−H)⁻.

### Example 181

### trans-4-{[3-(3-Quinolinyl)imidazo[1,2-b]pyridazin-6-yl]amino}cyclohexanol

MS: 360 (M+H)⁺.

### Example 182

### trans-4-{[3-(1H-Indol-5-yl)imidazo[1,2-b]pyridazin-6-yl]amino}cyclohexanol

¹H-NMR (DMSO-d₆) δ: 1.20-1.49 (4H, m), 1.88-2.01 (2H, m), 2.08-2.32 (2H, m), 3.41-3.68 (2H, m), 4.63 (1H, d, J=4.6 Hz), 6.44 (1H, s), 6.62 (1H, d, J=9.6 Hz), 6.88 (1H, d, J=6.5 Hz), 7.39 (1H, dd, J=2.7, 2.7 Hz), 7.45 (1H, d, J=8.6 Hz), 7.71 (1H, d, J=9.6 Hz), 7.77 (1H, dd, J=1.6, 8.7 Hz), 7.80 (1H, s), 8.61 (1H, s), 11.17 (1H, bs).

MS: 348 (M+H)⁺.

### Example 183

### trans-4-{[3-(3,4,5-Trimethoxyphenyl)imidazo[1,2-b]pyridazin-6-yl]amino}cyclohexanol

¹H-NMR (DMSO-d₆) δ: 1.14-1.31 (4H, m), 1.80-1.90 (2H, m), 2.03-2.10 (2H, m), 3.35-3.50 (1H, m), 3.71 (3H, d), 4.59 (1H, d, J=4.5 Hz), 6.65 (1H, d, J=9.6 Hz), 6.88 (1H, d, J=7.5 Hz), 7.47 (2H, s), 7.71 (1H, d, J=9.6 Hz), 7.88 (1H, s).

MS: 399 (M+H)⁺.

### Example 184

### trans-4-{[3-(5-Pyrimidinyl)imidazo[1,2-b]pyridazin-6-yl]amino}cyclohexanol

¹H-NMR (DMSO-d₆) δ: 1.23-1.30 (4H, m), 1.88 (2H, m), 2.12 (2H, m), 3.51 (2H, m), 4.60 (1H, d, J=4.1 Hz), 6.75 (1H, d, J=9.7 Hz), 7.13 (1H, d, J=6.6 Hz), 7.80 (1H, d, J=9.7 Hz), 8.13 (1H, s), 9.09 (1H, s), 9.58 (1H, s).

MS: 310 (M+H)⁺.

### Example 185

### trans-4-{[3-(6-Fluoro-3-pyridinyl)imidazo[1,2-b]pyridazin-6-yl]amino}cyclohexanol

¹H-NMR (DMSO-d₆) δ: 1.23-1.43 (4H, m), 1.88 (2H, m), 2.12 (2H, m), 3.51 (2H, m), 4.61 (1H, d, J=4.0 Hz), 6.71 (1H, d, J=9.7 Hz), 7.05 (1H, d, J=6.6 Hz), 7.31 (1H, dd, J=3.0, 8.6 Hz), 7.77 (1H, d, J=9.7 Hz), 7.99 (1H, s), 8.64-8.74 (1H, m), 9.09 (1H, s).

MS: 328 (M+H)⁺.

### Example 186

### trans-4-{[3-(2-Fluorophenyl)imidazo[1,2-b]pyridazin-6-yl]amino}cyclohexanol

MS: 327 (M+H)⁺.

### Example 187

### trans-4-([3-(3-Fluoro-4-methoxyphenyl)imidazo[1,2-b]pyridazin-6-yl]amino)cyclohexanol

¹H-NMR (DMSO-d₆) δ: 1.05-1.43 (4H, m), 1.76-1.83 (2H, m), 2.12-2.23 (2H, m), 3.51 (2H, m), 4.63 (1H, brs), 6.74 (1H, d, J=9.6 Hz), 7.02 (1H, d, J=6.6 Hz), 7.24 (1H, t, J=9.0 Hz), 7.74 (1H, d, J=9.6 Hz), 7.88-7.91 (2H, m), 8.23 (1H, dd, J=2.0, 13.9 Hz).

MS: 357 (M+H)⁺.

### Example 188

### trans-4-{[3-(2-Naphthyl)imidazo[1,2-b]pyridazin-6-yl]amino}cyclohexanol

¹H-NMR (DMSO-d₆) δ: 1.06-1.49 (4H, m), 1.94-1.98 (2H, m), 2.20-2.25 (2H, m), 3.45-3.83 (2H, m), 4.69 (1H, d, J=4.1 Hz), 6.73 (1H, d, J=9.7 Hz), 7.02 (1H, d, J=6.6 Hz), 7.48-7.58 (2H, m), 7.78 (1H, d, J=9.7 Hz), 7.86-7.96 (2H, m), 7.98 (1H, d, J=8.7 Hz), 8.06 (1H, s), 8.18 (1H, dd, J=1.6, 8.7 Hz), 8.99 (1H, s).

MS: 359 (M+H)⁺.

### Example 189

### trans-4-{[3-(2,3-Dihydro-1-benzofuran-5-yl)imidazo[1,2-b]pyridazin-6-yl]amino}cyclohexanol

MS: 351 (M+H)⁺.

### Example 190

### trans-4-{[3-(3,5-Dimethoxyphenyl)imidazo[1,2-b]pyridazin-6-yl]amino}cyclohexanol

¹H-NMR (DMSO-d₆) δ: 1.18-1.37 (4H, m), 1.79-1.92 (2H, m), 2.01-2.19 (2H, m), 3.39-3.54 (1H, m), 3.54-3.69 (1H, m), 3.82 (6H, s), 4.58 (1H, d, J=4.4 Hz), 6.47 (1H, t, J=2.2 Hz), 6.66 (1H, d, J=9.7 Hz), 6.91 (1H, d, J=7.1 Hz), 7.40 (2H, d, J=2.2 Hz), 7.72 (1H, d, J=9.7 Hz), 7.91 (1H, s).

MS: 369 (M+H)⁺.

### Example 191

### trans-4-{[3-(6-Quinolinyl)imidazo[1,2-b]pyridazin-6-yl]amino}cyclohexanol

¹H-NMR (DMSO-d₆): 1.18-1.48 (4H, m), 1.92-1.97 (2H, m), 2.18-2.23 (2 H, m), 3.47-3.66 (2H, m), 4.64 (1H, d, J=4.1 Hz), 6.74 (1H, d, J=9.7 Hz), 7.04 (1H, d, J=6.6 Hz), 7.46-7.65 (1H, m), 7.78 (1H, d, J=9.7 Hz), 8.06 (1H, d, J=9.0 Hz), 8.10 (1H, s), 8.28 (1H, d, J=7.8 Hz), 8.44 (1H, dd, J=1.8, 9.0 Hz), 8.89 (1H, dd, J=1.4, 4.2 Hz), 9.00 (1H, d, J=1.4 Hz).

### Example 192

### trans-4-({3-[3-(Benzyloxy)-4-methoxyphenyl]imidazo[1,2-b]pyridazin-6-yl}amino)cyclohexanol

¹H-NMR (DMSO-d₆) δ: 1.1-1.35 (4H, m), 1.78-1.95 (2H, m), 1.95-2.19 (2 H, m), 3.36-3.53 (1H, m), 3.53-3.71 (1H, m), 3.88 (3H, s), 4.60 (1H, d, J=4.3 Hz), 5.14 (2H, s), 6.63 (1H, d, J=9.7 Hz), 6.90 (1H, d, J=7.1 Hz), 7.12 (1H, d, J=8.5 Hz), 7.34-7.51 (5H, m), 7.62-7.83 (4H, m).

MS: 445 (M+H)⁺.

### Example 193

### trans-4-{[3-(3,4-Difluorophenyl)imidazo[1,2-b]pyridazin-6-yl]amino}cyclohexanol

¹H-NMR (DMSO-d₆) δ: 1.17-1.44 (4H, m), 1.79-2.03 (2H, m), 2.04-2.28 (2H, m), 3.39-3.65 (2H, m), 4.62 (1H, d, J=4.2 Hz), 6.70 (1H, d, J=9.6 Hz), 7.05 (1H, d, J=6.6 Hz), 7.52 (1H, dt, J=10.7, 8.8 Hz), 7.76 (1H, d, J=9.6 Hz), 7.80-8.03 (1H, m), 7.98 (1H, s), 8.48-8.52 (1H, m).

MS: 345 (M+H)⁺.

### Example 194

### trans-4-({3-[4-(Benzyloxy)phenyl]imidazo[1,2-b]pyridazin-6-yl}amino)cyclohexanol

MS: 415 (M+H)⁺.

### Example 195

### N-(4-{6-[(trans-4-Hydroxycyclohexyl)amino]imidazo[1,2-b]pyridazin-3-yl}phenyl)acetamide

MS: 366 (M+H)⁺.

### Example 196

### trans-4-{[3-(4-Bromophenyl)imidazo[1,2-b]pyridazin-6-yl]amino}cyclohexanol

MS: 389 (M+H)⁺.

### Example 197

### trans-4-({3-[4-(Benzyloxy)-3-fluorophenyl]imidazo[1,2-b]pyridazin-6-yl}amino)cyclohexanol

MS: 360 (M+H)⁺.

### Example 198

To a solution of 6-chloro-3-(4-pyridinyl)imidazo[1,2-b]pyridazine (50 mg) and trans-4-amino-4-methylcyclohexanol hydrochloride (27 mg) in toluene (5 mL) was added tris (dibenzylidenacetone) dipalladium chloroform complex (═Pd₂ dba₃.CHCl₃, 7 mg), 2,2′-Bis(diphenylphosphino)-1,1′-binaphthyl (=BINAP, 12 mg) and sodium tert-butoxide (87 mg). The mixture was stirred at 110° C. for 90 minutes under nitrogen atmosphere. The resultant was poured into water-dichloromethane (10% methanol), and extracted with dichloromethane. The organic phase was separated, washed with brine, and dried over sodium sulfate. Evaporation of the solvent gave a residue, which was purified by preparative TLC eluting with dichloromethane/methanol (10:1) to give trans-1-methyl-4-{[3-(4-pyridinyl)imidazo[1,2-b]pyridazin-6-yl]oxy}cyclohexanamine (3 mg).

¹H-NMR (DMSO-d₆) δ: 1.41 (3H, s), 1.37-1.86 (6H, m), 2.03-2.23 (2H, m), 5.00-5.11 (1H, m), 7.00 (1H, d, J=9.8 Hz), 8.11-8.17 (3H, m), 8.38 (1H, s), 8.67 (2H, d, J=6.1 Hz).

MS: 324 (M+H)⁺.

### Example 199

To a stirred mixture of 3-(4-pyridinyl)imidazo[1,2-b]pyridazin-6-amine (15.0 mg) and benzoyl chloride (15.0 mg) in dichloromethane (0.15 mL) was added N,N-diethylethanamine (14.4 mg) at ice-water bath temperature. The mixture was stirred for 1 hour at ambient temperature. The reaction mixture was poured into 1 M HCl and extracted with ethyl acetate. The combined organic layer was washed with brine, dried over magnesium sulfate, and concentrated in vacuo. The residue was purified by silica gel column chromatography eluting with chloroform/methanol (50:1 to 20:1) to give N-[3-(4-pyridinyl) imidazo[1,2-b]pyridazin-6-yl]benzamide (9.60 mg).

¹H-NMR (CDCl₃) δ: 7.30 (18, d, J=9.5 Hz), 7.38-7.53 (6H, m), 8.22 (1H, s), 8.23 (2H, d, J=8.5 Hz), 8.13 (2H, d, J=8.5 Hz), 8.59 (1H, bs).

MS: 316 (M+H)⁺.

### Example 200

To a solution of N-4-piperidinyl-3-(4-pyridinyl)imidazo-[1,2-b]pyridazin-6-amine trihydrochloride (70 mg) in methanol (560 μL) was added acetic anhydride (25 μL), which was stirred at 60° C. for 2 hours. To the mixture was added excess of triethylamine and acetic anhydride, which was stirred at 60° C. for 3 hours. The resultant was quenched by saturated NaHCO₃ aqueous solution, and extracted with dichloromethane. The organic layer was washed with brine, dried over sodium sulfate, filtered, and evaporated in vacuo. The residue was purified by column chromatography on silica gel to give N-(1-acetyl-4-piperidinyl)-3-(4-pyridinyl)imidazo[1,2-b]pyridazin-6-amine (57.7 mg) as a yellow powder.

MS: 337 (M+H)⁺.

### Example 201

To a stirred solution of ethyl 4-{[3-(4-pyridinyl)imidazo[1,2-b]pyridazin-6-yl]amino}benzoate (60 mg) in methanol (1.8 ml) was added 1M NaOH aqueous solution (0.5 mL) at ambient temperature. The reaction mixture was stirred at 25° C. for 15 hours. After all starting material had been consumed, as judged by TLC plate, the reaction mixture was diluted with ethyl acetate/water (10 mL:10 mL). The resulting mixture was poured into water and then neutralized by the addition of aqueous 1M HCl to pH 7. The resulting solution was extracted with ethyl acetate three times, the organic layers were combined, dried over magnesium sulfate, and concentrated in vacuo. The residue was purified by silica gel column chromatography eluting with chloroform/methanol (50:1 to 20:1) to give 4-{[3-(4-pyridinyl) imidazo[1,2-b]pyridazin-6-yl]amino}benzoic acid (23.5 mg).

¹H-NMR (DMSO-d₆) δ: 7.09 (1H, d, j=9.6 Hz), 7.62 (2H, d, J=8.5 Hz), 7.88 (2H, d, J=8.5 Hz), 7.99 (1H, d, J=9.6 Hz), 8.16 (2H, d, J=6.2 Hz), 8.20 (1H, s), 8.66 (2H, d, J=6.2 Hz).

### Example 202

To a solution of 6-chloro-3-(4-pyridinyl)imidazo[1,2-b]pyridazine (653 mg) and tetrahydro-2H-pyran-4-amine (100 mg) in toluene (8.7 mL) was added tris(dibenzylidenacetone) dipalladium chloroform complex (═Pd₂ dba₃.CHCl₃, 13 mg), 2,2′-Bis(diphenylphosphino)-1,1′-binaphthyl (=BINAP, 24 mg) and sodium tert-butoxide (125 mg). The mixture was stirred at 110° C. for 1.5 hours under nitrogen atmosphere. The reaction mixture was poured into water and extracted with 10% methanol in dichloromethane. The organic layer was washed with brine, dried over sodium sulfate. and evaporated in vacuo. The residue was purified by column chromatography on silica gel elution with chloroform/methanol (100:0 to 100:10). The fraction was concentrated, and dissolved into HCO₂NH₂ (514 μl) and sodium methoxide (117 mg) was added. After stirring at 100° C. for 2.5 hours, the reaction mixture was poured into water/dichloromethane. The resulting precipitates were collected by filtration to give trans-4-{[3-(4-pyridinyl)imidazo[1,2-b]pyridazin-6-yl]amino}cyclohexanecarboxamide (6 mg).

¹H-NMR (DMSO-d₆) δ: 1.08-2.04 (8H, m), 2.15-2.35 (1H, m), 3.80-3.99 (1H, m), 6.73 (1H, s), 6.90 (1H, d, J=9.7 Hz), 7.12 (1H, d, J=6.2 Hz), 7.24 (1H, s), 7.80 (1H, d, J=9.6 Hz), 8.16 (1H, s), 8.19 (2H, d, J=6.0 Hz), 8.60 (2H, d, J=6.0 Hz).

MS: 337 (M+H)⁺.

### Example 203

Triethylamine (113 μl) and ethyl chloroformate (18 μl) was added to a suspension of trans-N-[3-(4-pyridinyl)imidazo[1,2-b]pyridazin-6-yl]-1,4-cyclohexanediamine trihydrochloride (65 mg) in dichloromethane (1.3 ml). After stirring at ambient temperature for 1 hour, the reaction mixture was evaporated in vacuo. The residue was purified by preparative TLC eluting with dichloromethane/methanol (10:1) to give ethyl(trans-4-{[3-(4-pyridinyl)imidazo[1,2-b]pyridazin-6-yl]amino}cyclohexyl) carbamate (59 mg).

¹H-NMR (DMSO-d₆) δ: 1.29-1.56 (4H, m), 1.85-2.21 (4H, m), 3.26-3.64 (5H, m), 3.99 (2H, q, J=7.0 Hz), 6.79 (1H, d, J=9.7 Hz), 7.08 (1H, d, J=7.8 Hz), 7.25 (1H, d, J=6.5 Hz), 7.81 (1H, d, J=9.7 Hz), 8.17 (1H, s), 8.22 (2 H, d, J=5.8 Hz), 8.61 (2H, d, J=5.8 Hz).

MS: 381 (M+H)⁺.

### Example 204

A mixture of 6-chloro-3-(4-pyridinyl)imidazo[1,2-b]pyridazine (80 mg), 4-methylcylcohexylamine (231 μL), and 1,8-diazabicyclo[5.4.0]undec-7-ene (233 μL) in n-butyl alcohol (400 μL) was subjected to microwave irradiation at 150° C. for 2 hours. The resultant was dissolved in dimethylsulfoxide, which was purified by HPLC. The fractions containing desired compound were combined, evaporated and dried under reduced pressure to give N-(4-methylcyclohexyl)-3-(4-pyridinyl) imidazo[1,2-b]pyridazin-6-amine (18.7 mg) as a red powder.

MS: 308 (M+H)⁺.

In addition, fractions containing by-product obtained from this and additional example were combined, evaporated and dried under reduced pressure to give 6-butoxy-3-(4-pyridinyl) imidazo[1,2-b]pyridazine as a red powder.

¹H-NMR (DMSO-d₆) δ: 0.97 (3H, t, J=7.5 Hz), 1.50 (2H, sixtet, J=7.5 Hz), 1.8.3 (2H, quintet, J=8.5 Hz), 4.43 (2H, t, J=6.4 Hz), 7.03 (1H, d, J=9.6 Hz), 8.14 (1H₂O, J=9.5 Hz), 8.18 (2H, dd, J=1.5, 4.5 Hz), 8.38 (1H, s), 8.67 (2H, dd, J=1.5, 5.0 Hz).

### Example 205

To a solution of 6-chloro-3-(4-pyridinyl)imidazo[1,2-b]pyridazine (80 mg) and 1-methyl-4-piperidinamine (118.8 mg) in toluene (6.9 mL) was added tris(dibenzylidenacetone) dipalladium chloroform complex (Pd₂ dba₃-CHCl₃, 10.8 mg), 2,2′-Bis(diphenylphosphino)-1,1′-binaphthyl (=BINAP, 19.5 mg), and sodium tert-butoxide (150.0 mg), which was stirred at 110° C. for 2.5 hours under nitrogen atmosphere. The solvent was evaporated, the residue was dissolved in dimethylsulfoxide, and the resulting solution was desalted using solid-phase extraction cartridge. The solvent was evaporated and dissolved in dimethylsulfoxide (=DMSO), which was purified by HPLC. The fractions containing desired compound were combined, evaporated and dried under reduced pressure to give N-(1-methyl-4-piperidinyl)-3-(4-pyridinyl)imidazo[1,2-b]pyridazin-6-amine (19.0 mg) as a yellow powder.

¹H-NMR (DMSO-d₆) δ: 1.51 (2H, dd, J=11.3, 21.8 Hz), 1.95-2.18 (4H, m), 2.21 (3H, s), 2.70-2.84 (2H, m), 3.50-3.76 (1H, m), 6.77 (1H, d, J=9.8H z), 7.48 (1H, d, J=6.7 Hz), 7.81 (1H, d, J=9.8 Hz), 8.16 (1H, s), 8.19 (2H, 2.0, 5.0, J=2.0, 5.0 Hz), 8.60 (2H, dd, J=1.5, 5.0 Hz).

The following compound was obtained in a similar manner to that of Example 205.

### Example 206

### 3-{[3-(4-Pyridinyl)imidazo[1,2-b]pyridazin-6-yl]amino}-1-propanol

¹H-NMR (DMSO-d₆) δ: 1.82 (2H, quintet, J=6.5 Hz), 3.39 (2H, q, J=6.0 Hz), 3.56 (2H, q, J=6.0 Hz), 4.57 (1H, t, J=5.0 Hz), 6.78 (1H, d, J=10.0 Hz), 7.21 (1H, t, J=5.5 Hz), 7.80 (1H, d, J=9.5 Hz), 8.17 (1H, s), 8.23 (2H, d d, J=2.0, 5.0 Hz), 8.60 (2H, dd, J=1.5, 4.5 Hz).

MS: 270 (M+H)⁺.

### Example 207

A mixture of 6-chloro-3-(4-pyridinyl)imidazo[1,2-b]pyridazine (50 mg) and (2-phenylethyl)amine (164 μL) in cyclopentyl methyl ether (150 μL) was subjected to microwave irradiation at 180° C. for 1 hour. Evaporation of the volatile components gave a residue, which was purified by column chromatography on silica gel to give N-(2-phenylethyl)-3-(4-pyridinyl)imidazo[1,2-b]pyridazin-6-amine (68.1 mg) as a brown powder.

¹H-NMR (DMSO-d₆) δ: 2.97 (2H, t, J=6.5 Hz), 3.57 (2H, dt, J=6.5, 8.0 Hz), 6.79 (1H, d, J=9.6 Hz), 7.15-7.48 (6H, m), 7.81 (1H, d, J=9.7 Hz), 8.17 (1H, s), 8.21 (2H, dd, J=1.5, 4.5 Hz), 8.60 (2H, dd, J=1.5, 4.5 Hz).

MS: 316 (M+H)⁺.

The following compounds were obtained in a similar manner to that of Example 207.

### Example 208

### N-[2-(4-Morpholinyl)ethyl]-3-(4-pyridinyl)imidazo[1,2-b]pyridazin-6-amine

MS: 325 (M+H)⁺.

### Example 209

### N-[2-(1-Benzyl-4-piperidinyl)ethyl]-3-(4-pyridinyl)imidazo[1,2-b]pyridazin-6-amine

MS: 413 (M+H)⁺.

### Example 210

### N-(4-Piperidinylmethyl)₇₃-(4-pyridinyl)imidazo[1,2-b]pyridazin-6-amine

MS: 309 (M+H)⁺.

### Example 211

### 4-(2-{[3-(4-Pyridinyl)imidazo[1,2-b]pyridazin-6-yl]amino}ethyl)phenol

¹H-NMR (DMSO-d₆) δ: 2.84 (2H, t), 3.50 (2H, t), 6.72 (2H, d), 6.78 (1H, d), 7.11 (2H, d), 7.34 (1H, t), 7.81 (1H, d), 8.16 (1H, s), 8.22 (2H, d), 8.59 (2H, d), 9.23 (1H, bs).

MS: 332 (M+H)⁺.

### Example 212

### 2,6-Dichloro-4-(2-{[3-(4-pyridinyl)imidazo[1,2-b]pyridazin-6-yl]amino}ethyl)phenol

MS: 400 (M)⁺, 402 (M+2)⁺.

### Example 213

A mixture of trans-4-{[3-(2-chloro-4-pyridinyl)imidazo[1,2-b]pyridazin-6-yl]amino}cyclohexanol (30 mg), Zinc cyanide (20 mg), tetrakis(triphenylphosphine)palladium(0) (20 mg) in N,N-dimethylformamide (0.9 ml) was stirred at 180° C. for 2 hours. The reaction mixture was poured into water and extracted with ethyl acetate. The organic extract was washed with water twice and brine, dried over magnesium sulfate and concentrated in vacuo. The residue was purified by preparative TLC eluting with chloroform/methanol (10:1) to give 4-{6-[(trans-4-hydroxycyclohexyl)amino]imidazo[1,2-b]pyridazin-3-yl}-2-pyridinecarbonitrile (16 mg).

¹H-NMR (DMSO-d₆) δ: 1.02-1.49 (4H, m), 1.82-1.94 (2H, m), 2.12-2.17 (2H, m), 3.43-3.62 (2H, m), 4.63 (1H, d, J=3.9 Hz), 6.80 (1H, d, J=9.7 Hz), 7.21 (1H, d, J=6.6 Hz), 7.82 (1H, d, J=9.7 Hz), 7.27 (1H, s), 7.46 (1H, d d, J=1.4, 5.5 Hz), 7.74 (1H, d, J=5.5 Hz), 8.86 (1H, d, J=1.4 Hz).

MS: 335 (M+H)⁺.

### Example 214

To a solution of trans-4-(3-[2-(methylsulfinyl)-4-pyridinyl]imidazo[1,2-b]pyridazin-6-yl)amino)cyclohexanol (66 mg) in dichloromethane (5 ml) was added 3-chloroperoxybenzoic acid (95 mg), the mixture was stirred at 23° C. for 4 hours. The reaction mixture was quenched with NaHCO₃ aqueous solution and purified by preparative TLC eluting with chloroform/methanol (10:1) to give trans-4-({3-[2-(methylsulfonyl)-4-pyridinyl]imidazo[1,2-b]pyridazin-6-yl}amino)cyclohexanol (42 mg).

¹H-NMR (DMSO-d₆) δ: 1.18-1.55 (4H, m), 1.81-1.91 (2H, m), 2.06-2.11 (2H, m), 3.31 (3H, s), 3.31-3.78 (2H, m), 4.55 (1H, brs), 6.80 (1H, d, J=9.7 Hz), 7.12 (1H, d, J=6.6 Hz), 7.82 (1H, d, J=9.7 Hz), 7.90 (1H, dd, J=1.4, 5.5 Hz), 8.36 (1H, s), 8.76 (dH, d, J=5.5 Hz), 9.14 (1H, m).

MS: 388 (M+H)⁺.

### Example 215

To a solution of trans-4-({3-[2-(methylthio)-4-pyridinyl]imidazo[1,2-b]pyridazin-6-yl}amino)cyclohexanol (69 mg) in dichloromethane (5 ml) was added 3-chloroperoxybenzoic acid (49 mg), the mixture was stirred at 23° C. for 3 hours. The reaction mixture was quenched with NaHCO₃ aqueous solution and purified by preparative TLC eluting with chloroform/methanol (10:1) to give trans-4-({3-[2-(methylsulfinyl)-4-pyridinyl]imidazo[1,2-b]pyridazin-6-yl}amino)cyclohexanol (83.5 mg).

¹H-NMR (DMSO-d₆) δ: 1.05-1.57 (4H, m), 1.80-1.85 (2H, m), 2.06-2.10 (2H, m), 2.83 (3H, s), 3.31-3.80 (2H, m), 4.54 (1H, brs), 6.78 (1H, d, J=9.7 Hz), 7.07 (1H, d, J=6.6 Hz), 7.80 (1H, d, J=9.7 Hz), 8.20 (1H, dd, J=1.4, 5.5 Hz), 8.34 (1H, s), 8.67 (1H, d, J=5.5 Hz), 8.95 (1H, d, J=1.4 Hz).

MS: 372 (M+H)⁺.

### Example 216

To a solution of trans-4-{[3-(2-chloro-4-pyridinyl)imidazo[1,2-b]pyridazin-6-yl]amino}cyclohexanol (100 mg) in toluene (4 ml), N,N-dimethylformamide (1 ml) was added sodium methanethiolate (203 mg), the mixture was stirred at 150° C. for 4 hours. The reaction mixture was quenched by 1M HCl aqueous solution, neutralized with saturated NaHCO₃ aqueous solution, poured into water and extracted with ethyl acetate. The organic extract was washed with water twice and brine, dried over magnesium sulfate and concentrated in vacuo. The residue was purified by preparative TLC eluted with 15% methanol in chloroform to give trans-4-({3-[2-(methylthio)-4-pyridinyl]imidazo[1,2-b]pyridazin-6-yl}amino)cyclohexanol.

¹H-NMR (DMSO-d₆) δ: 1.20-1.50 (4H, m), 1.89-1.99 (2H, m), 2.10-2.16 (2H, m), 2.57 (3H, s), 3.44-3.63 (3H, m), 6.83 (1H, d, J=9.8 Hz), 7.22 (1H, d, J=6.8 Hz), 7.76 (1H, dd, J=1.4, 5.5 Hz), 7.83 (1H, d, J=9.8 Hz), 8.27 (1H, s), 8.33 (1H, d, J=1.4 Hz), 8.46 (1H, d, J=5.5 Hz).

MS: 356 (M+H)⁺.

The following compound was obtained in a similar manner to that of Example 216.

### Example 217

### trans-4-({3-[2-(Phenylthio)-4-pyridinyl]imidazo[1,2-b]pyridazin-6-yl}amino)cyclohexanol

¹H-NMR (DMSO-d₆) δ: 1.31-1.50 (4H, m), 1.88-1.94 (2H, m), 2.09-2.15 (2H, m), 6.78 (1H, d, J=9.6 Hz), 7.13 (1H, d, J=6.6 Hz), 7.42-7.59 (5H, m), 7.78-7.88 (2H, m), 8.12 (1H, s), 8.29 (1H, s), 8.38-8.41 (1H, m).

MS: 418.

### Example 218

To a solution of trans-4-{[3-(2-chloro-4-pyridinyl)imidazo[1,2-b]pyridazin-6-yl]amino}cyclohexanol (150 mg) in toluene (7.5 ml) and N,N-dimethylformamide (1.5 ml) was added 28% sodium methoxide in methanol (0.025 ml). The mixture was stirred at 150° C. for 13 hours. The reaction mixture was quenched by 1 M HCl aqueous solution, neutralized with saturated NaHCO₃ aqueous solution; poured into water and extracted with ethyl acetate. The organic extract was washed with water twice and brine, dried over magnesium sulfate and concentrated in vacuo. The residue was purified by preparative TLC eluted with 15% methanol in chloroform to give trans-4-{[3-(2-methoxy-4-pyridinyl)imidazo[1,2-b]pyridazin-6-yl]amino}cyclohexanol.

¹H-NMR (DMSO-d₆) δ: 1.25-1.39 (4H, m), 1.88-1.90 (2H, m), 2.05-2.15 (2H, m), 3.40-3.60 (2H, m), 3.89 (3H, s), 4.62 (1H, d, J=4.1 Hz), 6.74 (1H, d, J=9.7 Hz), 7.09 (1H, d, J=6.6 Hz), 7.68 (1H, dd, J=1.4, 5.6 Hz), 7.77 (1H, d, J=9.7 Hz), 7.86 (1H, s), 8.15 (1H, s), 8.17 (1H, d, J=5.6 Hz).

MS: 340 (M+H)⁺.

The following compound was obtained in a similar manner to that of Example 218.

### Example 219

### trans-4-{[3-(2-Phenoxy-4-pyridinyl)imidazo[1,2-b]pyridazin-6-yl]amino}cyclohexanol

¹H-NMR (DMSO-d₆) δ: 1.17-1.38 (4H, m), 1.86-1.99 (2H, m), 2.05-2.17 (2H, m), 3.48 (1H, brs), 4.58 (1H, d, J=4.1 Hz), 6.77 (1H, d, J=9.6 Hz), 7.10-7.38 (4H, m), 7.41-7.46 (2H, m), 7.78-7.86 (2H, m), 8.31-8.21 (3H, m).

MS: 402 (M+H)⁺, 424 (M+Na)⁺.

### Example 220

To a stirred solution of methyl 3-{6-[(trans-4-hydroxycyclohexyl)amino]imidazo[1,2-b]pyridazin-3-yl}benzoate (30 mg) in methanol (0.5 ml) was added 1 M NaOH aqueous solution (123 μL) at ambient temperature. The reaction mixture was stirred at 25° C. for 27 hours. After all starting material had been consumed, as judged by TLC plate, the reaction mixture was neutralized by the addition of aqueous 1M HCl aqueous solution to pH 7. The resulting solution was extracted with dichloromethane. The organic layer was dried over magnesium sulfate, and concentrated in vacuo to give 3-{6-[(trans-4-hydroxycyclohexyl)amino]imidazo[1,2-b]pyridazin-3-yl}benzoic acid (10 mg).

MS: 353 (M+H)⁺.

### Example 221

To a suspension of trans-N-[3-(4-pyridinyl)imidazo[1,2-b]pyridazin-6-yl]-1,4-cyclohexanediamine trihydrochloride (50 mg) in CH₃CN (1 ml) was added triethylamine (50 μl) and 35% aqueous HCHO (21 μl) under stirring at ambient temperature. After stirring at ambient temperature for 10 minutes, sodium acetoxy borohydride (25 mg) was added to the mixture. After stirring at ambient temperature. for overnight, the reaction mixture was poured into water and extracted with 10% methanol in dichloromethane, dried over sodium sulfate and evaporated in vacuo. The residue was purified by preparative TLC eluting with dichloromethane/methanol (10:1) to give trans-N,N-dimethyl-N′-[3-(4-pyridinyl)imidazo[1,2-b]pyridazin-6-yl]-1,4-cyclohexanediamine (15 mg).

¹H-NMR (DMSO-d₆) δ: 1.05-1.49 (5H, m), 1.83-2.00 (2H, m), 2.10-2.29 (2H, m), 2.21 (6H, s), 3.46-3.63 (1H, m), 6.75 (1H, d, J=9.8 Hz), 7.14 (1H, d, J=6.7 Hz), 7.79 (1H, d, J=9.7 Hz), 8.16 (1H, s), 8.21 (2H, d, J=6.1 Hz), 8.61 (2H, d, J=6.1 Hz).

MS: 337 (M+H)⁺.

### Example 222

To a suspension of trans-N-[3-(4-pyridinyl)imidazo[1,2-b]pyridazin-6-yl]-1,4-cyclohexanediamine trihydrochloride (50 mg) in dichloromethane (1 ml) was added triethylamine (70 μl) and phenylacetylchloride (19 μl) under stirring at 0° C. After stirring at ambient temperature for 2 hours, 10% methanol in dichloromethane was added to the reaction mixture and filtrated. The filtrate was evaporated in vacuo. The residue was purified by column chromatography on silica gel eluting with chloroform/methanol (100:2 to 100:25) to give 2-phenyl-N-(trans-4-{[3-(4-pyridinyl)imidazo[1,2-b]pyridazin-6-yl]amino}cyclohexyl)acetamide (30 mg).

¹H-NMR (DMSO-d₆) δ: 1.19-1.50 (4H, m), 1.79-1.99 (2H, m), 2.07-2.29 (2H, m), 3.22-3.45 (1H, m), 3.41 (2H, s), 3.2-3.69 (1H, m), 6.76 (1H, d, J=9.9 Hz), 7.17 (1H, d, J=6.6 Hz), 721-7.35 (5H, m), 7.80 (1H, d, J=9.9H z), 8.05 (1H, d, J=7.7 Hz), 8.16 (1H, s), 8.21 (2H, d, J=6.2 Hz), 8.61 (2H, d, J=6.2 Hz).

MS: 427 (M+H)⁺.

The following compound was obtained in a similar manner to that of Example 222.

### Example 223

### N-(trans-4-{[3-(4-Pyridinyl)imidazo[1,2-b]pyridazin-6-yl]amino}cyclohexyl)benzamide

MS: 413 (M+H)⁺.

### Example 224

To a solution of tert-butyl 4-{[3-(4-pyridinyl)imidazo[1,2-b]pyridazin-6-yl]amino}-1-piperidinecarboxylate (40 mg) in dichloromethane (1.2 mL) was added 4 M HCl in dioxane (507 μL) at 0° C., which was stirred at ambient temperature for 2 hours. The precipitate was filtered, and washed by diisopropyl ether to give N-4-piperidinyl-3-(4-pyridinyl)imidazo[1,2-b]pyridazin-6-amine trihydrochloride (38 mg) as a yellow powder.

MS: 295 (M+H)⁺.

The following compound was obtained in a similar manner to that of Example 224.

### Example 225

### N-3-Piperidinyl-3-(4-pyridinyl)imidazo[1,2-b]pyridazin-6-amine trihydrochloride

¹H-NMR (DMSO-d₆) δ: 1.45-2.10 (8H, m), 2.63-3.61 (4H, m), 7.14 (1H, d, J=9.8 Hz), 8.05 (1H, d, 8 Hz), 8.83-8.90 (2H, m), 9.12-9.15 (2H, m).

MS: 295.

### Example 226

To a solution of trans-N-[3-(4-pyridinyl)imidazo[1,2-b]pyridazin-6-yl]-1,4-cyclohexanediamine trihydrochloride (23 mg) in dichloroformate (0.46 ml) was added triethylamine (40 μl) and methyl chloroformate (5.1 μl) at 0° C. After stirring at 0° C. for 40 minutes, the mixture was quenched with water and extracted with ethyl acetate. The organic layer was washed with water and brine, dried over magnesium sulfate, and evaporated in vacuo. The residue was purified by preparative TLC eluting with dichloromethane/methanol (10:1) to give methyl(trans-4-{[3-(4-pyridinyl)imidazo[1,2-b]pyridazin-6-yl]amino}cyclohexyl)carbamate (20 mg) as an pale yellow solid.

¹H-NMR (CDCl₃-CD₃OD (5:1)) δ: 1.33-1.55 (4H, m), 1.73-1.92 (2H, m), 2.13-2.38 (2H, m), 3.75-4.04 (2H, m), 3.93 (3H, s), 6.92 (1H, d, J=8.4 Hz), 7.74 (1H, d, J=8.1 Hz), 8.28 (1H, s), 8.67-8.75 (2H, m), 8.80-8.91 (2H, m).

MS: 367 (M+H)⁺.

### Example 227

The suspension of trans-N-[3-(4-pyridinyl)imidazo[1,2-b]pyridazin-6-yl]-1,4-cyclohexanediamine trihydrochloride (20 mg), KOCN (8 mg) and sodium acetate (8 mg) in N,N-dimethylformamide (0.4 ml) was stirred at 50° C. for 4 hours. After cooling to ambient temperature, the reaction mixture was purified by preparative TLC eluting with (dichloromethane:methanol=10:1) to give N-(trans-4-{[3-(4-pyridinyl)imidazo[1,2-b]pyridazin-6-yl]amino}cyclohexyl)urea (6 mg).

¹H-NMR (DMSO-d₆) δ: 1.16-1.42 (4H, m), 1.82-1.99 (2H, m), 2.11-2.24 (2H, m), 3.49-3.69 (1H, m), 3.29-3.46 (3H, m), 5.93 (1H, d, J=7.8 Hz), 6.76 (1H, d, J=9.7 Hz), 7.17 (1H, d, J=6.7 Hz), 7.80 (1H, d, J=9.7 Hz), 8.16 (1H, s), 8.21 (2H, d, J=6.1 Hz), 8.60 (2H, d, J=6.1 Hz).

MS: 352 (M+H)⁺.

The following compounds were obtained in a similar manner to that of Example 227

### Example 228

### N-Phenyl-3-{[3-(4-pyridinyl)imidazo[1,2-b]pyridazin-6-yl]amino}-1-piperidinecarboxamide

¹H-NMR (DMSO-d₆) δ: 1.47-1.75 (2H, m), 1.82 (1H, brs), 2.11 (1H, brs), 2.84-2.94 (1H, m), 3.00-3.18 (1H, m), 4.78 (1H, brs), 3.95 (1H, d, J=12.8 Hz), 4.31 (1H, d, J=12.8 Hz), 6.81 (1H, c, J=9.8 Hz), 6.92 (1H, t, J=7.3 Hz), 7.47 (1H, d, J=7.9 Hz), 7.82 (1H, d, J=9.8 Hz), 8.14-8.19 (3H, m), 8.48-8.51 (3H, m).

MS: 414 (M+H)⁺, 436 (M+Na)⁺.

### Example 229

### N-Methyl-3-{[3-(4-pyridinyl)imidazo[1,2-b]pyridazin-6-yl]amino}-1-piperidinecarboxamide

¹H-NMR (DMSO-d₆) δ: 1.23 (1H, m), 1.49 (2H, m), 1.78 (1H, m), 2.08 (1H, m), 2.66-2.72 (1H, m), 2.88 (1H, m), 3.71 (2H, m), 4.25 (1H, d, J=4.4 Hz), 6.43 (1H, d, J=4.2 Hz), 6.80 (1H, d, J=9.8 Hz), 7.15 (1H, d, J=6.6 Hz), 7.80-7.83 (1H, m), 8.16-8.22 (3H, m), 8.57-8.59 (2H, m).

MS: 352 (M+H)⁺, 374 (M+Na)⁺.

### Example 230

To a suspension of trans-4-{[3-(4-pyridinyl)imidazo[1,2-b]pyridazin-6-yl]amino}cyclohexanol (100 mg) in dichloromethane (2 ml) was added Dess-Martin periodinane (206 mg). After stirring for overnight, the mixture was poured into saturated aqueous NaHCO₃ aqueous solution and extracted with 10% methanol in chloroform, dried over sodium sulfate and evaporated in vacuo. The residue was purified by column chromatography on silica gel eluting with chloroform/methanol (100:2 to 10:1) to give 4-{[3-(4-pyridinyl)imidazo[1,2-b]pyridazin-6-yl]amino}cyclohexanone as an pale yellow solid (46 mg).

¹H-NMR (DMSO-d₆) δ: 1.71-1.90 (2H, m), 2.23-2.62 (6H, m), 4.09-4.24 (1H, m), 6.81 (1H, d, J=10.0 Hz), 7.31 (1H, d, J=6.0 Hz), 7.84 (1H, d, J=9.5 Hz), 8.19 (1H, s), 8.23 (2H, d, J=6.0 Hz), 8.63 (2H, d, J=6.0 Hz).

MS: 308 (M+H)⁺.

### Example 231

4 M HCl aqueous solution in 1,4-dioxane (428 μl) was added to a solution of tert-butyl, (trans-4-{[3-(4-pyridinyl)imidazo[1,2-b]pyridazin-6-yl]amino}cyclohexyl)carbamate (70 mg) in ethyl acetate (0.7 ml) under stirring at 0° C. After stirring at ambient temperature for 4 hours, the reaction mixture was evaporated in vacuo. Resulting precipitates were collected by filtration and washed with diisopropylether to give trans-N-[3-(4-pyridinyl)imidazo[1,2-b]pyridazin-6-yl]-1,4-cyclohexanediamine trihydrochloride (40 mg) as an yellow solid.

¹H-NMR (DMSO-d₆) δ: 1.26-1.47 (2H, m), 1.61-1.83 (2H, m), 2.00-2.22 (4H, m), 3.03-3.22 (1H, m), 3.58-3.78 (1H, m), 4.70 (3H, br), 7.03 (1H, d, J=9.5 Hz), 7.65-7.76 (1H, m), 7.98 (1H, d, J=9.5 Hz), 8.18 (2H, br), 8.69 (1H, s), 8.76 (2H, d, J=7.0 Hz), 8.93 (2H, d, J=7.0 Hz).

MS: 309 (Mfree+H)⁺.

### Example 232

A mixture of 6-chloro-3-(4-pyridinyl)imidazo[1,2-b]pyridazine (50 mg) and [(1-ethyl-2-pyrrolidinyl)methyl]amine (94.8 μL) in ethylene glycol dimethyl ether (=DME, 150 μL) was subjected to microwave irradiation at 150° C. for 90 minutes. Evaporation of the volatile components gave a residue, which was purified by column chromatography on silica gel to give N-[(1-ethyl-2-pyrrolidinyl)methyl]-3-(4-pyridinyl)imidazo[1,2-b]pyridazin-6-amine (63.1 mg) as a yellow powder.

¹H-NMR (DMSO-d₆) δ: 1.05 (3H, t, J=7.0 Hz), 1.51-1.98 (4H, m), 2.02-2.37 (2H, m), 2.61-2.79 (1H, m), 2.82-3.22 (3H, m), 3.51-3.69 (1H, m), 6.85 (1H, d, J=9.7 Hz), 7.18 (1H, t, J=5.6 Hz), 7.79 (1H, d, J=9.7 Hz), 8.15 (1H, s), 8.20 (2H, dd, J=1.5, 4.5 Hz), 8.59 (2H, dd, J=1.5, 4.5 Hz).

MS: 323 (M+H)⁴″.

The following compounds were obtained in a similar manner to that of Example 232.

### Example 233

### N-[2-(1-Piperidinyl)ethyl]-3-(4-pyridinyl)imidazo[1,2-b]pyridazin-6-amine

MS: 323 (M+H)⁺.

### Example 234

### N,N-Dimethyl-N′-[3-(4-pyridinyl)imidazo[1,2-b]pyridazin-6-yl]-1,2-ethanediamine

MS: 283 (M+H)⁺.

### Example 235

To a stirred mixture of 6-chloro-3-(4-pyridinyl)imidazo[1,2-b]pyridazine (80 mg) and (4-pyridinylmethyl)amine (113 mg) in toluene (4 ml) were added sodium tert-butoxide (150 mg), (R)-2,2′-Bis(diphenylphosphino)-1,1′-binaphthyl (=(R)-BINAP, 19.4 mg), and tris(dibenzylideneacetone) dipalladium chloroform complex (═Pd₂(dba)₃.CHCl₃, 10.8 mg) at ambient temperature. The reaction mixture was subjected to microwave irradiation at 100° C. for 1 hour. After all starting material had been consumed, as judged by TLC plate, the reaction mixture was cooled to ambient temperature and diluted with ethyl acetate/water (10 mL/10 mL). The resulting mixture was acidified with 1 M HCl aqueous solution to pH 2 and extracted with Ethyl acetate. The aqueous phase was then adjusted to pH 8 with 2 M NaOH aqueous solution. The resulting solution was extracted with ethyl acetate three times, the organic layers were combined, dried over magnesium sulfate, and concentrated in vacuo. The residue was purified by silica gel column chromatography eluting with chloroform/methanol (40:1 to 20:1) to give 3-(4-pyridinyl)-N-(4-pyridinylmethyl)imidazo[1,2-b]pyridazin-6-amine.

¹H-NMR (DMSO-d₆) δ: 4.57 (2H, d, J=5.7 Hz), 6.92 (1H, d, J=9.6 Hz), 7.44 (2H, d, J=5.8 Hz), 7.86 (2H, d, J=5.8 Hz), 7.88 (1H, d, J=9.6 Hz), 7.98 (1H, t, J=5.7 Hz), 8.13 (1H, s), 8.48 (2H, d, J=4.8 Hz), 8.55 (2H, d, J=4.8H z).

MS: 303 (M+H)⁺.

The following compounds were obtained in a similar manner to that of Example 235.

### Example 236

### N-(2,4-Dichloro-5-methoxyphenyl)-3-(4-pyridinyl)imidazo[1,2-b]pyridazin-6-amine

MS: 388.

### Example 237

### N-[(1S,2R)-2-Methylcyclohexyl]-3-(4-pyridinyl)imidazo[1,2-b]pyridazin-6-amine

¹H-NMR (DMSO-d₆) δ: 0.92 (3H, d, J=7.0 Hz), 1.31-1.73 (7H, m), 1.78-1.95 (1H, m), 2.03-2.21 (1H, m), 3.92-4.10 (1H, m), 6.91 (1H, d, J=7.5 Hz), 6.94 (1H, d, J=9.8 Hz), 7.78 (1H, d, J=9.8 Hz), 8.15 (1H, s), 8.19 (2H, d, J=6.4 Hz), 8.60 (2H, d, J=6.4 Hz).

MS: 308 (M+H)⁺.

### Example 238

### N-[2-Chloro-5-(tetrahydro-2H-pyran-2-yloxy)phenyl]-3-(4-pyridinyl)imidazo[1,2-b]pyridazin-6-amine

MS: 422 (M+H)⁺.

### Example 239

### (3-{[3-(4-Pyridinyl)imidazo[1,2-b]pyridazin-6-yl]amino}phenyl)methanol

MS: 318 (M+H)⁺.

### Example 240

### N-(5-Methoxy-2-methylphenyl)-3-(4-pyridinyl)imidazo[1,2-b]pyridazin-6-amine

MS: 332 (M+H)⁺.

### Example 241

### N-(2-Methylphenyl)-3-(4-pyridinyl)imidazo[1,2-b]pyridazin-6-amine

MS: 302 (M+H)⁺.

### Example 242

### (1R,2R,3S,5s)-5-{[3-(4-Pyridinyl)imidazo[1,2-b]pyridazin-6-yl]amino}-2-adamantanol

¹H-NMR (DMSO-d₆) δ: 1.45-2.28 (13H, m), 4.02 (1H, s), 4.24 (1H, s), 6.42 (1H, d, J=9.6 Hz), 7.68 (1H, d, J=9.6 Hz), 7.96 (1H, s), 8.07 (2H, d, J=6.0 Hz), 8.67 (2H, d, J=6.0 Hz).

MS: 364 (M+H)⁺.

### (1R,2S,3S,5s)-5-{[3-(4-Pyridinyl)imidazo[1,2-b]pyridazin-6-yl]amino}-2-adamantanol

¹H-NMR (DMSO-d₆) δ: 1.47-2.62 (13H, m), 3.88 (1H, s), 4.24 (1H, s), 6.41 (1H, d, J=9.6 Hz), 7.66 (1H, d, J=9.6 Hz), 7.96 (1H, s), 8.12 (2H, d, J=6.2 Hz), 8.64 (2H, d, J=6.2 Hz).

MS: 364 (M+H)⁺.

### Example 243

### trans-4-{[7-Methyl-3-(4-pyridinyl)imidazo[1,2-b]pyridazin-6-yl]amino}cyclohexanol

MS: 324 (M+H)⁺.

### Example 244

### trans-4-{[8-Methyl-3-(4-pyridinyl)imidazo[1,2-b]pyridazin-6-yl]amino}cyclohexanol

¹H-NMR (DMSO-d₆) δ: 1.28-1.56 (4H, m), 1.82-1.99 (2H, m), 2.07-2.21 (2H, m), 2.23 (3H, s), 3.38-3.59 (1H, m), 3.62-3.83 (1H, m), 4.62 (1H, d, J=4.6 Hz), 6.17 (1H, d, J=7.0 Hz), 7.69 (1H, s), 8.13 (1H, s), 8.20 (2H, d, J=6.2 Hz), 8.58 (2H, d, J=6.2 Hz).

MS: 324 (M+H)⁺.

### Example 245

### N-(3-Methylcyclohexyl)-3-(4-pyridinyl)imidazo[1,2-b]pyridazin-6-amine

MS: 308 (M+H)⁺.

### Example 246

### N-[1-(2-Pyrazinyl)-4-piperidinyl]-3-(4-pyridinyl)imidazo[1,2-b]pyridazin-6-amine

¹H-NMR (DMSO-d₆) δ: 1.48-1.58 (2H, m), 2.16-2.20 (2H, m), 3.21-3.28 (2H, m), 3.93-4.05 (1H, m), 4.29-4.33 (2H, m), 6.78 (1H, d, J=9.8 Hz), 7.26 (1H, d, J=6.6 Hz), 7.82-7.84 (2H, m), 8.09-8.10 (1H, m), 8.19 (1H, s), 8.21-8.22 (2H, m), 8.40 (1H, d, J=1.4 Hz), 8.62-8.63 (2H, m).

MS: 373 (M+H)⁺.

### Example 247

### N-(1-Phenyl-4-piperidinyl)-3-(4-pyridinyl)imidazo[1,2-b]pyridazin-6-amine

MS: 371 (M+H)⁺.

### Example 248

### N-2-Adamantyl-3-(4-pyridinyl)imidazo[1,2-b]pyridazin-6-amine

¹H-NMR (DMSO-d₆) δ: 1.48-2.25 (14H, m), 3.92-4.02 (1H, m), 6.97 (1H, d, J=9.6 Hz), 7.14 (1H, d, J=6.1 Hz), 7.80 (1H, d, J=9.6 Hz), 8.16 (1H, s), 8.19 (2H, d, J=6.1 Hz), 8.58 (2H, d, J=6.1 Hz).

MS: 346 (M+H)⁺.

### Example 249

### N-1-Adamantyl-3-(4-pyridinyl)imidazo[1,2-b]pyridazin-6-amine

¹H-NMR (DMSO-d₆) δ: 1.73 (6H, s), 2.15 (9H, s), 6.80 (1H, d, J=9.6 Hz), 6.81 (1H, d, J=4.5 Hz), 7.86 (1H, d, J=9.6 Hz), 8.12 (1H, s), 8.19 (2H, d, J=4.8 Hz), 8.60 (2H, d, J=4.8 Hz).

MS: 346 (M+H)⁺.

### Example 250

### N-[(1S,2R)-2-Ethylcyclohexyl]-3-(4-pyridinyl)imidazo[1,2-b]pyridazin-6-amine

MS: 322 (M+H)⁺.

### Example 251

### 3-(4-Pyridinyl)-N-[2-(2-pyridinyl)ethyl]imidazo[1,2-b]pyridazin-6-amine

MS: 317 (M+H)⁺.

### Example 252

To a mixture of N-4-piperidinyl-3-(4-pyridinyl)imidazo[1,2-b]Pyridazin-6-amine trihydrochloride (60 mg) and K₂CO₃ (123.6 mg) in acetone (900 μL) was added methyl chloroformate (69 μL), which was stirred under reflux for 8 hours. To the resultant was added water. The mixture was extracted with dichloromethane. The organic layer was washed with brine, dried over sodium sulfate, filtered, and evaporated in vacuo. The residue was purified by column chromatography on silica gel to give methyl 4-{[3-(4-pyridinyl)imidazo[1,2-b]pyridazin-6-yl]amino}-1-piperidinecarboxylate (35.3 mg) as a white powder.

MS: 353 (M+H)⁺.

### Example 253

To a mixture of N-4-piperidinyl-3-(4-pyridinyl)imidazo[1,2-b]pyridazin-6-amine trihydrochloride (70 mg) and formic acid (196 μL) was added acetic anhydride (19.6 μL), which was stirred at 60° C. for 4 hours. To the mixture was added excess of ethyl formate and triethylamine, which was stirred at 60° C. for 4 hours. To the resultant was added water. The mixture was extracted with dichloromethane. The organic layer was washed with brine, dried over sodium sulfate, filtered and evaporated in vacuo. The residue was purified by column chromatography on silica gel to give 4-{[3-(4-pyridinyl)imidazo[1,2-b]pyridazin-6-yl]amino}-1-piperidinecarbaldehyde (5.1 mg) as a yellow powder.

¹H-NMR (DMSO-d₆) δ: 1.17-1.61 (2H, m), 2.00-2.26 (2H, m), 2.83-3.09 (1H, m), 3.14-3.48 (1H, m), 3.64-4.24 (3H, m), 6.78 (1H, d, J=9.7 Hz), 7.28 (1H, d, J=6.6 Hz), 7.84 (1H, d, J=9.7 Hz), 8.04 (1H, s), 8.14-8.24 (3H, m), 8.63 (2H, dd, J=1.4, 4.7 Hz).

MS: 323 (M+H)⁺.

### Example 254

trans-4-{[3-(4-Pyridinyl)imidazo[1,2-b]pyridazin-6-yl]amino}cyclohexanecarboxylic acid bis(trifluoroacetate) (61 mg) was dissolved into N,N-dimethylformamide (1.2 ml), and 1-hydroxybenzotriazole (═HOBT, 16 mg), 1-ethyl-3-(3-dimethylaminopropyl)carbodiimide (=EDCI, 23 mg) and triethylamine (47 μl) was added to the solution.

Then, methylamine hydrochloride (8 mg) was added to the mixture. After stirring at ambient temperature for overnight, the reaction mixture was poured into water, and extracted with 10% methanol in dichloromethane. The organic layer was dried over magnesium sulfate and evaporated in vacuo. The residue was purified by preparative TLC eluting with dichloromethane/methanol (10:1) to give trans-N-methyl-4-([3-(4-pyridinyl) imidazo[1,2-b]pyridazin-6-yl]amino)cyclohexanecarboxamide (3 mg).

¹H-NMR (DMSO-d₆) δ: 1.50-2.00 (8H, m), 2.12-2.31 (1H, m), 2.57 (3H, d, J=4.5 Hz), 3.88-4.01 (1H, m), 6.91 (1H, d, J=9.7 Hz), 7.13 (1H, d, J=6.3 Hz), 7.69 (1H, d, J=4.5 Hz), 7.80 (1H, d, J=9.7 Hz), 8.16 (1H, s), 8.19 (2H, d, J=6.3 Hz), 8.60 (2H, d, J=6.3 Hz).

MS: 351 (M+H)⁺.

The following compound was obtained in a similar manner to that of Example 254

### Example 255

### trans-N,N-Dimethyl-4-{[3-(4-pyridinyl)imidazo[1,2-b]pyridazin-6-yl]amino}cyclohexanecarboxamide

MS: 365 (M+H)⁺.

### Example 256

A mixture of 6-chloro-3-(4-pyridinyl)imidazo[1,2-b]pyridazine (80 mg), 2M methylamine in tetrahydrofuran (867 μL), and methylamine hydrochloride (234 mg) was subjected to microwave irradiation at 160° C. for 6 hours. To the resultant was added saturated NaHCO₃ aqueous solution The mixture was extracted with dichloromethane. The organic layer was washed with brine, dried over sodium sulfate, filtered, and evaporated in vacuo. The residue was purified by column chromatography on silica gel to give N-methyl-3-(4-pyridinyl)imidazo[1,2-b]pyridazin-6-amine (14 mg) as a white powder.

MS: 226 (M+H)⁺.

### Example 257

A mixture of 6-chloro-3-(4-pyridinyl)imidazo[1,2-b]pyridazine (50 mg) and (1S,2S)-2-amino-1-phenyl-1,3-propanediol (362.5 mg) was subjected to microwave irradiation at 180° C. for 30 minutes. The resultant mixture was dissolved in dimethylsulfoxide, which was purified by HPLC to give (1S,2S)-1-phenyl-2-{[3-(4-pyridinyl)imidazo[1,2-b]pyridazin-6-yl]amino}-1,3-propanediol (25.1 mg) as a yellow powder.

MS: 362 (M+H)⁺.

The following compound was obtained in a similar manner to that of Example 257.

### Example 258

### (1R,2S,4R)-4-([3-(4-Pyridinyl)imidazo[1,2-b]pyridazin-6-yl]-amino)-1,2-cyclopentanediol

¹H-NMR (DMSO-d₆) δ: 1.55-1.71 (2H, m), 2.26-2.41 (2H, m), 3.84-4.11 (3H, m), 4.48-4.53 (2H, m), 6.82 (1H, d, J=9.7 Hz), 7.25 (1H, d, J=6.5 Hz), 7.78 (1H, d, J=9.7 Hz), 8.14 (1H, s), 8.21 (2H, d, J=6.3 Hz), 8.61 (2H, d, J=6.3 Hz).

MS: 312 (M+H)⁺.

### Example 259

### N-[(3-exo)-8-Benzyl-8-azabicyolo[3.2.1]oct-3-yl]-3-(4-pyridinyl)imidazo[1,2-b]pyridazin-6-amine

MS: 411 (M+H)⁺.

### Example 260

### (1R,2R)-1-Phenyl-2-{[3-(4-pyridinyl)imidazo[1,2-b]pyridazin-6-yl]amino}-1,3-propanediol

MS: 382 (M+H)⁺.

### Example 261

1 M methyl magnesium bromide in tetrahydrofuran (0.14 ml) was added dropwise to a solution of 4-{[3-(4-pyridinyl)imidazo[1,2-b]pyridazin-6-yl]amino}cyclohexanone (22 mg) in tetrahydrofuran (1 ml). After stirring at ambient temperature for 2 hours, additional 1 M methyl magnesium bromide in tetrahydrofuran (0.14 ml) was added dropwise, then stirred at ambient temperature for 2 hours. 1M HCl aqueous solution was added to the reaction mixture, and saturated NaHCO₃ aqueous solution was added until pH 8. Then, extracted with 10% methanol in chloroform, dried over sodium sulfate and evaporated in vacuo. The residue was purified by preparative TLC developing with dichloromethane/methanol (10:1) to give 1-methyl-4-{[3-(4-pyridinyl)imidazo[1,2-b]pyridazin-6-yl]amino}cyclohexanol (2 mg).

¹H-NMR (DMSO-d₆) δ: 0.97-1.31 (2H, m), 1.17 (3H, s), 1.41-1.71 (3H, m), 1.81-2.01 (2H, m), 2.73-2.97 (1H, m), 3.48-3.88 (1H, m), 6.81 (1H, d, J=9.9 Hz), 7.09 (1H, d, J=5.5 Hz), 7.79 (1H, d, J=9.9 Hz), 8.16 (1H, s), 8.20 (2H, d, J=6.2 Hz), 8.60 (2H, d, J=6.2 Hz), 8.61 (1H, s).

MS: 324 (M+H)⁺.

### Example 262

To a mixture of 6-chloro-3-(4-pyridinyl)imidazo[1,2-b]pyridazine (80 mg) and 1,8-diazabicyclo[5.4.0]undec-7-ene (233 μL) in N-methyl-2-pyrrolidinone (240 μL) was added 4-tert-butylcyclohexylamine (310 μL). The mixture was subjected to microwave irradiation at 150° C. for 2.5 hours. To the resultant was added water. The mixture was extracted with ethyl acetate/n-hexane (4:1). The organic layer was washed brine, dried over sodium sulfate, filtered, and evaporated in vacuo. The residue was purified by preparative TLC to give N-(4-tert-butylcyclohexyl)-3-(4-pyridinyl)imidazo[1,2-b]pyridazin-6-amine (34.1 mg) as a yellow amorphous.

MS: 350 (M+H)⁺.

### Example 263

To a stirred solution of trans-4-{[3-(3,4-dimethoxyphenyl) imidazo[1,2-b]pyridazin-6-yl]amino}cyclohexanol (37 mg) in dichloromethane (1 ml) was added dropwise 1.0 M BBr₃ in dichloromethane (1.004 mL) at 0° C. The reaction mixture was stirred at this temperature for 1 hour. The reaction mixture was neutralized with 1 M NaOH aqueous solution and extracted with chloroform/methanol (5:1). The organic layers was dried over magnesium sulfate, and concentrated in vacuo. The residue was purified by silica gel column chromatography eluting with chloroform/methanol (5:1) to give 4-{6-[(trans-4-hydroxycyclohexyl)amino]imidazo[1,2-b]pyridazin-3-yl}-1,2-benzenediol (10 mg).

¹H-NMR (DMSO-d₆) δ: 1.20-1.38 (4H, m), 1.85-1.91 (2H, m), 2.09-2.15 (2H, m), 3.41-3.62 (2H, m), 4.60 (1H, brs), 6.59 (1H, d, J=9.6 Hz), 6.79-6.85 (2H, m), 7.46-7.50 (1H, m), 7.55 (1H, d, J=2.4 Hz), 7.63 (1H, s), 7.67 (1H, d, J=9.6 Hz), 8.87 (1H, brs), 9.14 (1H, brs).

MS: 341 (M+H)⁺.

The following compounds were obtained in a similar manner to that of Example 263.

### Example 264

### 3-methyl-4-{[3-(4-pyridinyl)imidazo[1,2-b]pyridazin-6-yl]amino}phenol

¹H-NMR (DMSO-d₆) δ: 2.17 (3H, s), 6.68-6.75 (2H, m), 6.96 (1H, d, J=9.6 Hz), 7.31 (1H, d, J=8.4 Hz), 7.92 (1H, d, J=9.6 Hz), 8.02-8.04 (2H, m), 8.20 (1H, s), 8.44-8.46 (2H, m), 8.58 (1H, s), 9.30 (1H, s).

MS: 318 (M+H)⁺.

### Example 265

### 3-{[3-(4-Pyridinyl)imidazo[1,2-b]pyridazin-6-yl]amino}-5-(trifluoromethyl)phenol

MS: 372 (M+H)⁺.

### Example 266

A mixture of trans-4-{[3-(2-chloropyridin-4-yl)imidazo[1,2-b]pyridazin-6-yl]amino}cyclohexanol (100 mg), palladium acetate (═Pd(OAc)₂, 6.5 mg), 1,1′-bis(diphenylphosphino) ferrocene (=DPPF, 16.1 mg) NaHCO₃ (29 mg) in methanol (1 ml) was stirred at reflux under CO for 4 hours. The reaction mixture was poured into water and extracted with ethyl acetate. The organic extract was washed with water twice and brine, dried over magnesium sulfate and concentrated in vacuo. The residue was purified by silica gel column chromatography eluted with chloroform/methanol (100:0 to 95:5) to give methyl 4-{6-[(trans-4-hydroxycyclohexyl)amino]imidazo[1,2-b]pyridazin-3-yl}-2-Pyridinecarboxylate (40 mg).

¹H-NMR (DMSO-d₆) δ: 1.23-1.41 (6H, m), 1.87-1.89 (2H, m), 2.12-2.13 (2H, m), 3.48-3.50 (1H, m), 3.66-3.70 (1H, m), 3.94 (3H, s), 4.60-4.63 (1H, m).

MS: 390 (M+Na)⁺.

### Example 267

A mixture of trans-4-{[3-(2-chloropyridin-4-yl)imidazo[1,2-b]pyridazin-6-yl]amino}cyclohexanol (200 mg), phenylboronic acid (121 mg), tetrakis(triphenylphosphine) palladium(0) (20 mg), sodium carbonate (98 mg) in toluene (1.6 ml), ethanol (0.4 ml) and water (0.8 ml) was stirred at 100° C. for 1 hours. The reaction mixture was poured into water and extracted with ethyl acetate. The organic extract was washed with water twice and brine, dried over magnesium sulfate and concentrated in vacuo. The residue was purified by silica gel column chromatography eluted with chloroform/methanol (100:0 to 95:5) to give trans-4-{[3-(2-phenyl-4-pyridinyl)imidazo[1,2-b]pyridazin-6-yl]amino}cyclohexanol (67 mg).

¹H-NMR (DMSO-d₆) δ: 1.25-1.34 (4H, m), 1.86 (2H, brs), 2.12 (2H, brs), 3.47 (1H, brs), 3.75 (1H, brs), 4.60 (1H, d, J=3.8 Hz), 7.77 (1H, d, J=9.6 Hz), 7.08 (1H, d, J=6.6 Hz), 7.47-7.58 (3H, m), 7.80 (1H, d, J=9.6 Hz), 8.09-8.18 (2H, m), 8.28 (1H, s), 8.69 (1H, d, J=5.3 Hz), 8.79 (1H, s).

MS: 386.

### Example 268

A mixture of trans-4-{[3-(2-chloropyridin-4-yl)imidazo[1,2-b]pyridazin-6-yl]amino}cyclohexanol (30 mg), aniline (24 mg), palladium acetate (═Pd(OAc)₂, 1.9 mg), 2,2′-bis (diphenylphosphino)1,1′-binaphthyl (=BINAP, 5.4 mg) sodium tert-butoxide (33 mg) in N,N-dimethylformamide (1.5 ml) was stirred at 130° C. for 1.5 hours. The reaction mixture was poured into water and extracted with ethyl acetate. The organic extract was washed with water twice and brine, dried over magnesium sulfate and concentrated in vacuo. The residue was purified by silica gel column chromatography eluted with chloroform/methanol (100:0 to 95:5) to give trans-4-{[3-(2-anilino-4-pyridinyl)imidazo[1,2-b]pyridazin-6-yl]amino}cyclohexanol (15.2 mg).

¹H-NMR (DMSO-d₆) δ: 1.24-2.30 (4H, m), 1.82-1.90 (2H, m), 2.05-2.12 (2H, m), 3.65 (2H, brs), 4.54 (1H, d, J=4.0 Hz), 6.73 (1H, d, J=9.6 Hz), 6.89-7.00 (2H, m), 7.22-7.30 (2H, m), 7.51-7.79 (5H, m), 7.93 (1H, s), 8.19 (1H, d, J=5.5 Hz), 8.99 (1H, m).

MS: 401.

### Example 269

A solution of trans-4-{[3-(2-chloropyridin-4-yl)imidazo[1,2-b]pyridazin-6-yl]amino}cyclohexanol (39 mg) in 6M HCl/dioxane (3 ml) was stirred at 100° C. for 48 hours. The reaction mixture was concentrated in vacuo. The residue, was purified by silica gel column chromatography eluting with chloroform:methanol=20:1 to 10:1) to give 4-{6-[(trans-4-hydroxycyclohexyl)amino]imidazo[1,2-b]pyridazin-3-yl}-2 (1H)-pyridinone (7.3 mg).

¹H-NMR (DMSO-d₆) δ: 1.15-1.46 (4H, m), 1.86-1.91 (2H, m), 2.09-2.14 (2H, m), 3.50 (2H, brs), 6.74-6.79 (3H, m), 7.13 (1H, d, J=6.7 Hz), 7.35 (1H, d, J=7.0 hz), 7.55 (1H, s), 7.77 (1H, d, J=9.6 Hz), 8.08 (1H, s).

MS: 401.

The following compound was obtained in a similar manner to that of Example 269

### Example 270

### 5-{6-[(trans-4-Hydroxycyclohexyl)amino]imidazo[1,2-b]pyridazin-3-yl}-2 (1H)-pyridinone

¹H-NMR (DMSO-d₆) δ: 1.20-1.46 (4H, m), 1.83-1.90 (2H, m), 2.03-2.09 (2H, m), 3.51 (2H, brs), 4.62 (1H, s), 6.45 (1H, d, J=9.6 Hz), 6.62 (1H, d, J=9.6 Hz), 6.92 (1H, d, J=7.2 Hz), 7.70 (1H, d, J=9.8 Hz), 7.75 (1H, s), 8.03 (1H, dd, J=2.5, 9.6 Hz), 8.44 (1H, d, J=2.5 Hz), 12.00 (1H, brs).

MS: 326 (M+H)⁺, 348 (M+Na)⁺.

### Example 271

To a mixture of benzenthiol (238 mg) and sodium hydride (78 mg) in 1,3-dimethyl-2-imidazolidinone (=DMI, 8.75 ml) was added 6-chloro-3-(4-pyridinyl)-imidazo[1,2-b]pyridazine (100 mg), the mixture was stirred at rt. The reaction mixture was poured into water and extracted with ethyl acetate. The organic extract was washed with water twice and brine, dried over magnesium sulfate and concentrated in vacuo. The residue was purified by silica gel column chromatography eluted with chloroform/methanol (9:1) to 6-(phenylthio)-3-(4-pyridinyl) imidazo[1,2-b]pyridazine (81 mg).

¹H-NMR (DMSO-d₆) δ: 7.35 (1H, d, J=9.6 Hz), 7.58-7.78 (7H, m), 8.15 (1H, d, J=9.6 Hz), 8.39-8.41 (2H, m), 8.45 (1H, s).

MS: 305 (M+H)⁺.

The following compounds were obtained in a similar manner to that of Example 271

### Example 272

### 6-(Cyclohexyloxy)-3-(4-pyridinyl)imidazo[1,2-b]pyridazine

¹H-NMR (DMSO-d₆) δ: 1.21-1.62 (7H, m), 1.78 (2H, brs), 2.12 (2H, m), 1.17 (1H, brs), 6.99 (1H, d, J=9.6 Hz), 8.10-8.17 (3H, m), 8.37 (1H, s), 8.65-8.68 (2H, m).

MS: 295 (M+H)⁺, 317 (M+Na)⁺.

### Example 273

### 6-Phenoxy-3-(4-pyridinyl)imidazo[1,2-b]pyridazine

¹H-NMR (DMSO-d₆) δ: 7.32 (1H, d, J=9.6 Hz), 7.39-7.43 (3H, m), 7.52-7.60 (1H, m), 7.55 (1H, d, J=6.6 Hz), 7.82-7.85 (2H, m), 8.32 (1H, d, J=9.6 Hz), 8.41-8.45 (3H, m).

MS: 289.

### Example 274

To a solution of methyl 4-{6-[(trans-4-hydroxycyclohexyl)amino]imidazo[1,2-b]pyridazin-3-yl}pyridine-2-carboxylate (17 mg) in ethanol (1 mL) was added sodium borohydride (5.2 mg) at ambient temperature. The resulting mixture was stirred at ambient temperature. The reaction mixture was poured into water and extracted with ethyl acetate. The organic extract was washed with water twice and brine, dried over magnesium sulfate and concentrated in vacuo. The residue was purified by silica gel column chromatography eluted with chloroform/methanol (9:1) to trans-4-({3-[2-(hydroxymethyl)-4-pyridinyl]imidazo[1,2-b]pyridazin-6-yl}amino)cyclohexanol (15 mg).

¹H-NMR (DMSO-d₆) δ: 1.23-1.43 (4H, m), 1.84-1.90 (2H, m), 2.08-2.15 (2H, m), 3.67 (2H, brs), 4.58 (1H, brs), 4.62 (2H, s), 5.42 (1H, brs), 6.75 (1H, d, J=9.6 Hz), 7.05 (1H, d, J=6.6 Hz), 7.78 (1H, d, J=9.6 Hz), 8.04 (1H, d, J=4.0 Hz), 8.14 (1H, s), 8.40 (1H, s), 8.51 (1H, d, J=5.4 Hz).

MS: 340 (M+H)⁺, 362 (M+Na)⁺.

### Example 275

Aniline (96.2 mg) and lithium 1, 1, 1, 3,3,3-hexamethyldisilazan-2-ide (94.2 μl) were mixed in tetrahydrofuran (1.5 mL) under argon atmosphere and were stirred at 0° C. for 30 minutes. To the reaction mixture, methyl 6-[(trans-4-hydroxycyclohexyl)amino]imidazo[1,2-b]pyridazine-3-carboxylate was added at 0° C. The temperature was raised to ambient temperature, and the whole mixture was stirred for additional 3 hours at 25° C. The mixture was poured into saturated NH₄Cl and stirred for 30 minutes. The resulting solution was extracted with ethyl acetate three times, the organic layers were combined, dried over magnesium sulfate, and concentrated in vacuo. The residue was purified by silica gel column chromatography eluting with chloroform/methanol (50:1 to 10:1) to give 6-[(trans-4-hydroxycyclohexyl)amino]-N-phenylimidazo[1,2-b]pyridazine-3-carboxamide (17.5 mg).

¹H-NMR (DMSO-d₆) δ: 1.15-1.42 (4H, m), 1.76-2.23 (4H, m), 3.37-3.59 (1H, m), 3.60-3.83 (1H, m), 4.62 (1H, d), 6.87 (1H, d), 7.16 (1H, dd), 7.33-7.53 (3H, m), 7.70 (2H, d), 7.88 (1H, d), 8.02 (1H, s), 10.80 (1H, s).

MS: 374 (M+Na)⁺.

### Example 276

To a stirred mixture of 2-(3-{[3-(4-pyridinyl)imidazo[1,2-b]pyridazin-6-yl]amino}benzyl)-1H-isoindole-1,3(2H)-dione (120 mg) and in ethanol/tetrahydrofuran (total 2.4 ml) were added hydrazine hydrate (67.3 mg) at ambient temperature. The reaction mixture was refluxed for 5 hours. After all starting material had been consumed, as judged by TLC plate, the reaction mixture was cooled to ambient temperature. The resulting solution was concentrated in vacuo. The residue was purified by silica gel column chromatography eluting with chloroform/methanol (98:2 to 90:10) to give N-[3-(aminomethyl)phenyl]-3-(4-pyridinyl)imidazo[1,2-b]pyridazin-6-amine (23.1 mg).

¹H-NMR (DMSO-d₆) δ: 3.77 (2H, s), 7.00-7.08 (2H, m), 7.33 (1H, dd), 7.40-7.48 (1H, m), 7.82 (1H, s), 8.00 (1H, d), 8.17 (2H, d), 8.22 (1H, s), 8.66 (2H, d), 9.52 (1H, bs).

MS: 317 (M+H)⁺.

### Example 277

To a stirred mixture of N-(trans-4-ethoxycyclohexyl)-3-(4-pyridinyl)imidazo[1,2-b]pyridazin-6-amine (100.0 mg) and NaHCO₃ (49.8 mg) in methanol (1.5 ml) were added Br₂ (47.4 mg) at ambient temperature. The reaction mixture was stirred for 3 hours at the same temperature. After all starting material had been consumed, as judged by TLC plate, the resulting solution was filtrated. The filtration was extracted with ethyl acetate three times, the organic layers were combined, dried over magnesium sulfate, and concentrated in vacuo. The residue was purified by silica gel column chromatography eluting with chloroform/methanol (50:1 to 20:1) to give 2-bromo-N-(trans-4-ethoxycyclohexyl)-3-(4-pyridinyl) imidazo[1,2-b]pyridazin-6-amine (36.4 mg).

¹H-NMR (DMSO-d₆) δ: 1.10 (3H, t), 1.19-1.39 (4H, m), 1.87-2.17 (4H, m), 3.16-3.40 (1H, m), 3.40-3.56 (1H, m), 3.45 (2H, q), 6.79 (1H, d), 7.17 (1H, d), 7.75 (1H, d), 7.94 (2H, d), 8.70 (2H, d).

MS: 416 (M)⁺, 418 (M+2)⁺.

### Example 278

To a stirred mixture of 2-bromo-N-(trans-4-ethoxycyclohexyl)-3-(4-pyridinyl)imidazo[1,2-b]pyridazin-6-amine (30.0 mg) and Zinc cyanide (16.9 mg) in 1,3-dimethyl-2-imidazolidinone (=DMI, 0.90 ml) were added tetrakis(triphenylphosphine) palladium(0) (25.0 mg) at ambient temperature. The reaction mixture was stirred for 3 hours at 140° C. After all starting material had been consumed, as judged by TLC plate, the reaction mixture was cooled to ambient temperature and diluted with ethyl acetate/water (10 mL/10 mL). The resulting solution was extracted with ethyl acetate three times, the organic layers were combined, dried over magnesium sulfate, and concentrated in vacuo. The residue was purified by silica gel column chromatography eluting with chloroform/methanol (50:1 to 10:1) to give 6-[(trans-4-ethoxycyclohexyl)amino]-3-(4-pyridinyl) imidazo[1,2-b]pyridazine-2-carbonitrile (13.2 mg).

¹H-NMR (CDCl₃) δ: 1.23 (3H, t), 1.23-1.59 (4H, m), 2.03-2.39 (4H, m), 3.20-3.42 (1H, m), 3.54 (2H, q), 3.60-3.82 (1H, m), 4.77 (1H, d), 6.71 (1H, d), 7.72 (1H, d), 8.29 (2H, d), 8.79 (2H, d).

MS: 363 (M+H)⁺.

### Example 279

To a stirred mixture of 6-[(trans-4-ethoxycyclohexyl)amino]-3-(4-pyridinyl)imidazo[1,2-b]pyridazine-2-carbonitrile (10.0 mg) and 5M NaOH aqueous solution (49.5 μl) in ethanol/dimethylsulfoxide (0.4 ml/0.4 ml) were added 31% H₂O₂ aqueous solution (27.6 μl) at ambient temperature. The reaction mixture was stirred for 3 hours at 50° C. After all starting material had been consumed, as judged by TLC plate, the reaction mixture was cooled to ambient temperature and quenched with 1M HCl aqueous solution. The resulting mixture was neutralized with 0.1 M NaOH aqueous solution to PH 7. Then, the resulting solution was extracted with ethyl acetate three times, the organic layers were combined, dried over magnesium sulfate, and concentrated in vacuo. The residue was purified by silica gel column chromatography eluting with chloroform/ethanol (50:1 to 10:1) to give 6-[(trans-4-ethoxycyclohexyl)amino]-(4-pyridinyl)imidazo[1,2-b]pyridazine-2-carboxamide (4.56 mg).

¹H-NMR (CDCl₃) δ: 1.22 (3H, t), 1.23-1.45 (4H, m), 2.02-2.24 (4H, m), 3.22-3.36 (1H, m), 3.53 (2H, q), 3.50-3.70 (1H, m), 4.46 (1H, d), 5.46 (1 H, s), 6.61 (1H, d), 7.31 (1H, s), 7.66 (1H, d), 8.15 (2H, d), 8.70 (2H, d).

MS: 403 (M+Na)⁺.

### Example 280

To a stirred mixture of trans-4-({3-[(E)-2-phenylvinyl]imidazo[1,2-b]pyridazin-6-yl}amino)cyclohexanol (100.0 mg) and cyclohexene (1.0 ml) in ethanol/tetrahydrofuran (3.0 ml/3.0 ml) were added palladium hydroxide (═Pd(OH)₂, 50.0 mg) at ambient temperature. The reaction mixture was stirred at 80° C. for 5 hours. After all starting material had been consumed, as judged by TLC plate, the resulting solution was filtrated. After filtration, the reaction mixture was evaporated in vacuo. The residue was purified by silica gel column chromatography eluting with chloroform/methanol (98:2 to 90:10) to give trans-4-{[3-(2-phenylethyl)imidazo[1,2-b]pyridazin-6-yl]amino}cyclohexanol (45.6 mg).

¹H-NMR (DMSO-d₆) δ: 1.10-1.42 (4H, m), 1.81-2.22 (4H, m), 2.87-3.28 (4H, m), 3.32-3.69 (2H, m), 4.57 (1H, d), 6.56 (1H, d), 6.75 (1H, d), 7.14-7.38 (6H, m), 7.60 (1H, d).

MS: 337 (M+H)⁺.

### Example 281

To a stirred solution of N,6-[(1S)-1-phenylethyl]imidazo[1,2-b]pyridazine-3,6-diamine (50 mg) in dichloromethane (0.5 ml) was added isocyanatobenzene (0.0214 mL) at 0° C. The reaction mixture was stirred at this temperature for 2 hours. The reaction mixture was concentrated in vacuo. The residue was purified by washing with ethyl acetate/n-hexane to give 1-phenyl-3-(6-{[(1S)-1-phenylethyl]amino}imidazo[1,2-b]pyridazin-3-yl)urea (55 mg).

MS: 395 (M+Na)⁺.

The following compound was obtained in a similar manner to that of Example 281.

### Example 282

### 1-(2-Chlorophenyl)-3-(6-{[(18)-1-phenylethyl]amino}imidazo[1,2-b]pyridazin-3-yl)urea

MS: 429 (M+Na)⁺.

### Example 283

To a stirred solution of N-6-[(1S)-1-phenylethyl]imidazo[1,2-b]pyridazine-3,6-diamine (80 mg) in dichloromethane (0.8 ml) were added triethylamine (0.176 mL) and benzenesulfonyl chloride (83.7 mg) at 0° C. The reaction mixture was stirred at the same temperature for 4 hours. The reaction mixture was quenched with water and extracted with dichloromethane. The organic layers was dried over magnesium sulfate, and concentrated in vacuo. The residue was purified by silica gel column chromatography eluting with chloroform/methanol (9:1) to give N-(6-{[(1S)-1-phenylethyl]amino}imidazo[1,2-b]pyridazin-3-yl)benzenesulfonamide (27 mg).

MS: 394 (M+H)⁺.

TABLE 1
The following compounds (Example 284-Example 361)
were obtained in a similar manner to that of Example 98.

| Ex | Str. |
|---|---|
| 284 | 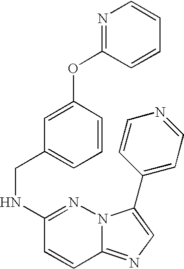 |
| 285 | 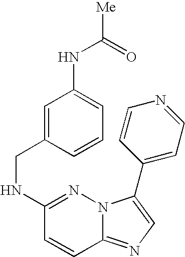 |
| 286 | 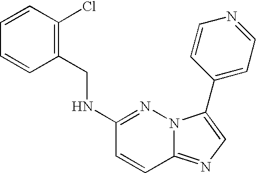 |
| 287 | 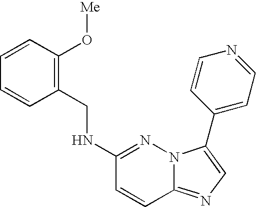 |
| 288 | 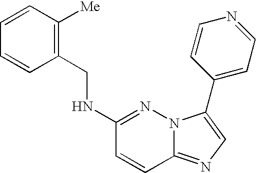 |
| 289 | 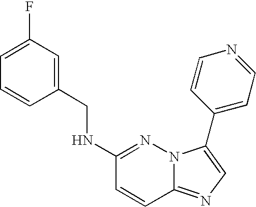 |
| 290 | 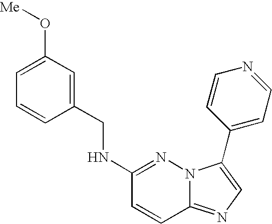 |
| 291 | 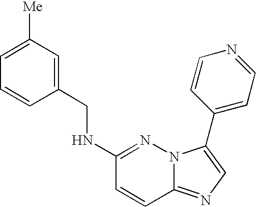 |
| 292 | 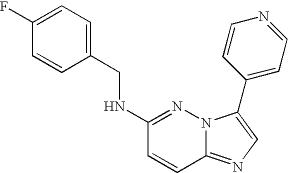 |
| 293 | 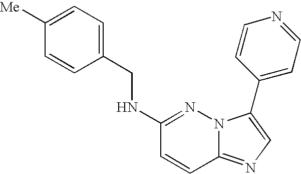 |
| 294 | 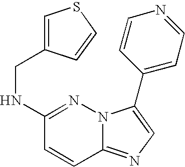 |
| 295 | 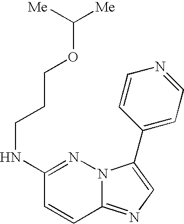 |
| 296 | 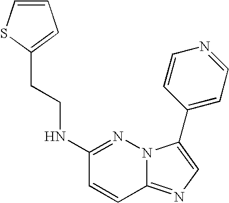 |
| 297 | 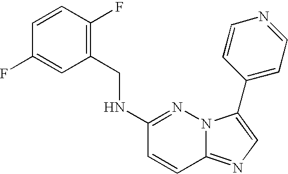 |
| 298 | 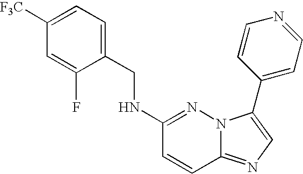 |
| 299 | 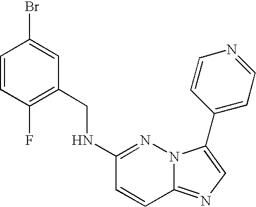 |
| 300 | 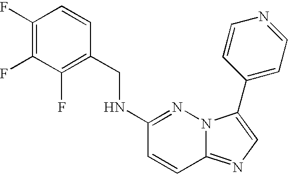 |
| 301 | 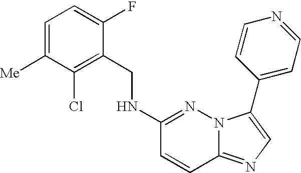 |
| 302 | 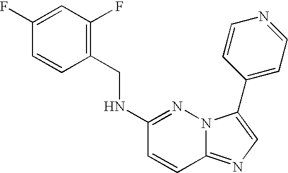 |
| 303 | 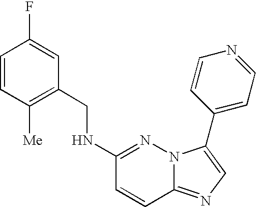 |
| 304 | 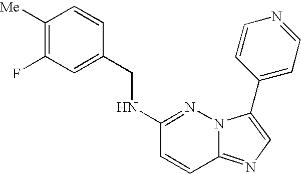 |
| 305 | 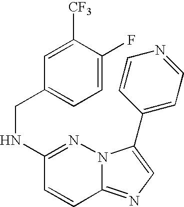 |
| 306 | 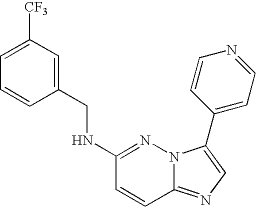 |
| 307 | 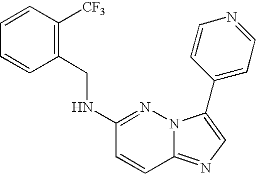 |
| 308 | 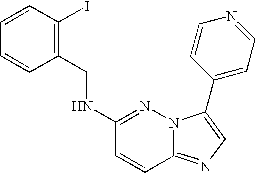 |
| 309 | 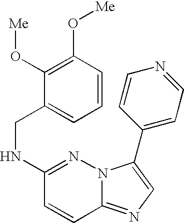 |
| 310 | 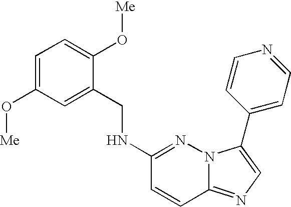 |
| 311 | 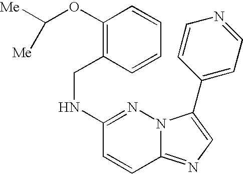 |
| 312 | 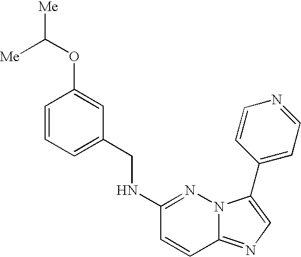 |
| 313 | 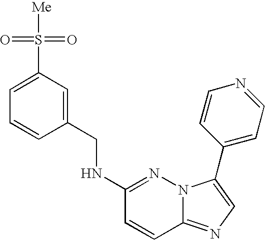 |
| 314 | 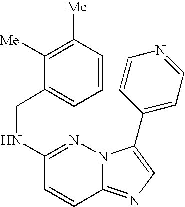 |
| 315 | 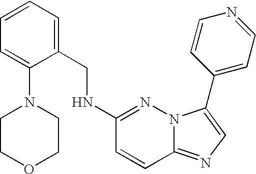 |
| 316 | 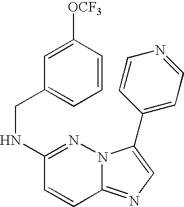 |
| 317 | 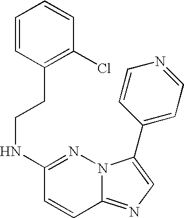 |
| 318 | 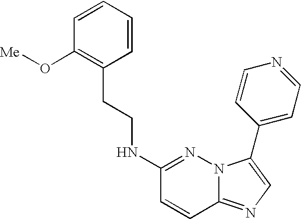 |
| 319 | 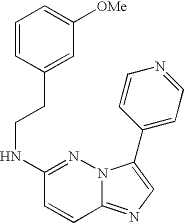 |
| 320 | 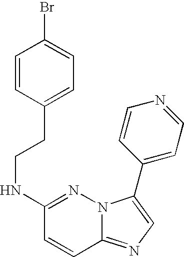 |
| 321 | 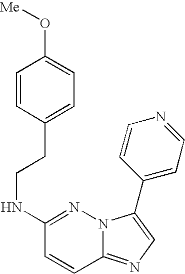 |
| 322 | 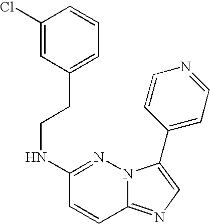 |
| 323 | 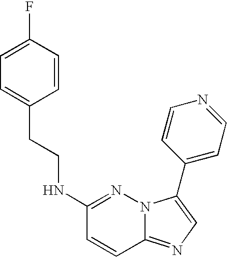 |
| 324 | 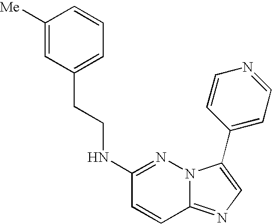 |
| 325 | 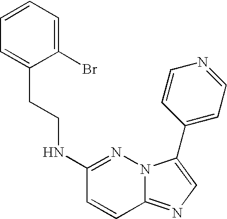 |
| 326 | 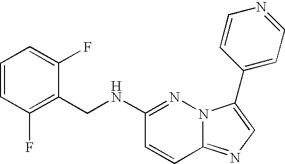 |
| 327 | 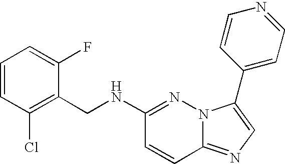 |
| 328 | 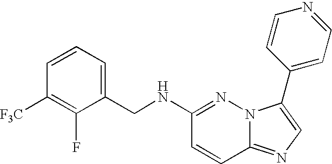 |
| 329 | 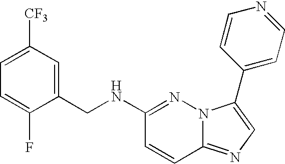 |
| 330 | 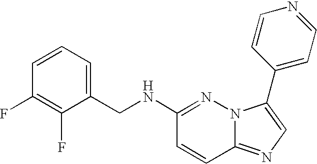 |
| 331 | 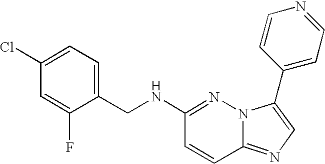 |
| 332 | 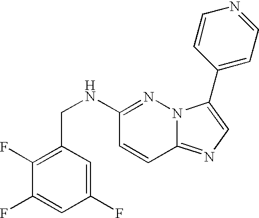 |
| 333 | 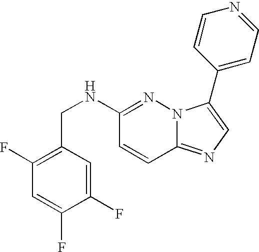 |
| 334 | 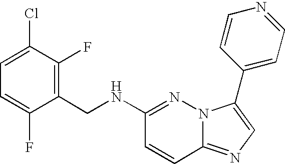 |
| 335 | 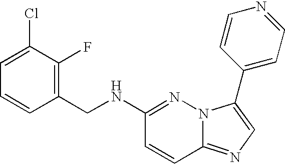 |
| 336 | 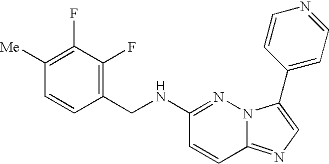 |
| 337 | 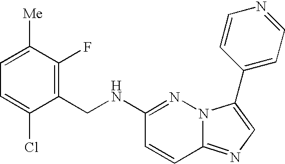 |
| 338 | 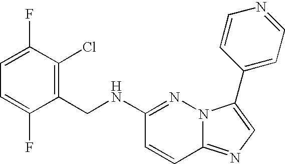 |
| 339 | 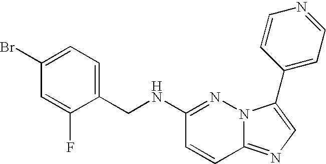 |
| 340 | 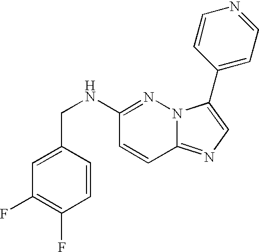 |
| 341 | 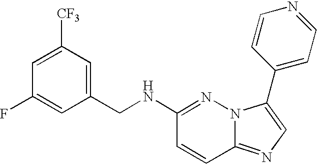 |
| 342 | 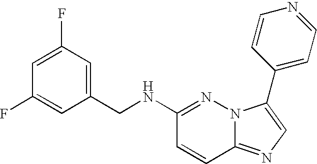 |
| 343 | 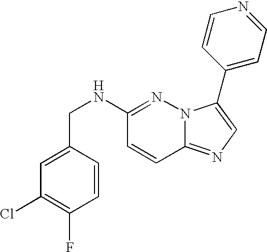 |
| 344 | 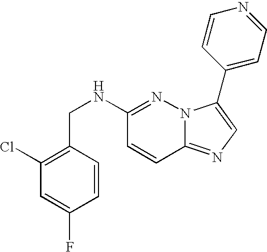 |
| 345 | 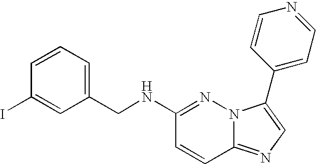 |
| 346 | 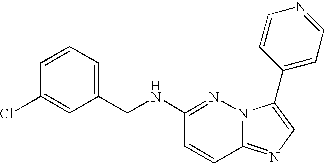 |
| 347 | 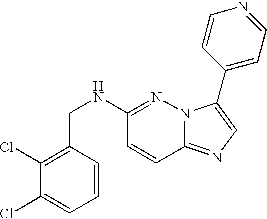 |
| 348 | 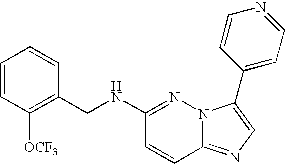 |
| 349 | 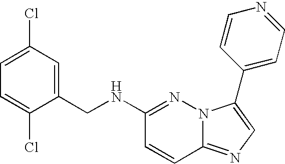 |
| 350 | 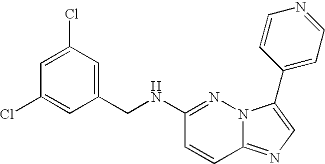 |
| 351 | 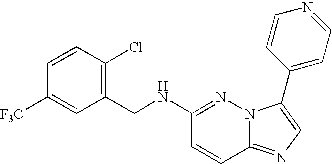 |
| 352 | 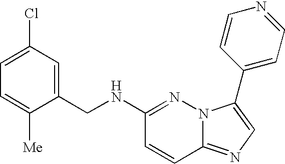 |
| 353 | 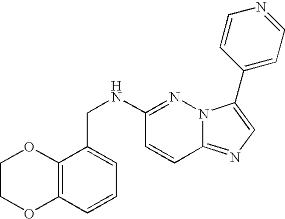 |
| 354 | 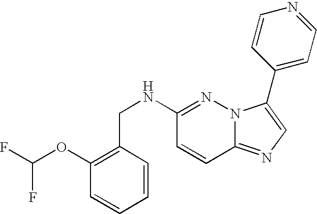 |
| 355 | 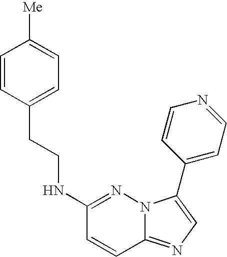 |
| 356 | 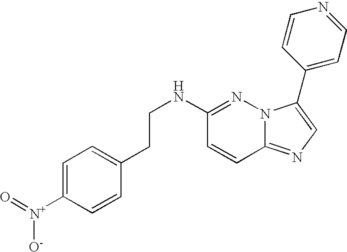 |
| 357 | 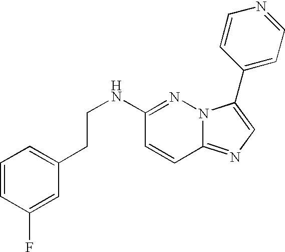 |
| 358 | 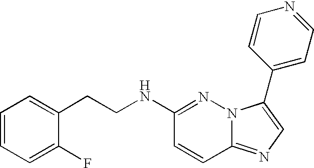 |
| 359 | 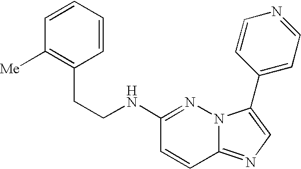 |
| 360 | 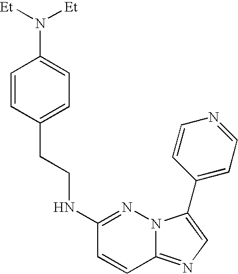 |
| 361 | 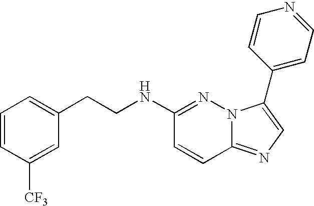 |

Ex: Example,
Str.: Structure.

TABLE 2

| Ex | MS |
|---|---|
| 284 | 395 |
| 285 | 359 |
| 286 | 336 |
| 287 | 332 |
| 288 | 316 |
| 289 | 320 |
| 290 | 332 |
| 291 | 316 |
| 292 | 320 |
| 293 | 316 |
| 294 | 308 |
| 295 | 312 |
| 296 | 322 |
| 297 | 338 |
| 298 | 388 |
| 299 | 398 |
| 300 | 356 |
| 301 | 368 |
| 302 | 338 |
| 303 | 334 |
| 304 | 334 |
| 305 | 388 |
| 306 | 370 |
| 307 | 370 |
| 308 | 428 |
| 309 | 362 |
| 310 | 362 |
| 311 | 360 |
| 312 | 360 |
| 313 | 380 |
| 314 | 330 |
| 309 | 362 |
| 310 | 362 |
| 311 | 360 |
| 312 | 360 |
| 313 | 380 |
| 314 | 330 |
| 315 | 387 |
| 316 | 386 |
| 317 | 350 |
| 318 | 346 |
| 319 | 346 |
| 320 | 394 |
| 321 | 346 |
| 322 | 350 |
| 323 | 334 |
| 324 | 330 |
| 325 | 394 |
| 326 | 338 |
| 327 | 354 |
| 328 | 388 |
| 329 | 388 |
| 330 | 338 |
| 331 | 354 |
| 332 | 356 |
| 333 | 356 |
| 334 | 372 |
| 335 | 354 |
| 336 | 352 |
| 337 | 368 |
| 338 | 372 |
| 339 | 398 |
| 340 | 338 |
| 341 | 388 |
| 342 | 338 |
| 343 | 354 |
| 344 | 354 |
| 345 | 428 |
| 346 | 336 |
| 347 | 370 |
| 348 | 386 |
| 349 | 370 |
| 350 | 370 |
| 351 | 404 |
| 352 | 350 |
| 353 | 360 |
| 354 | 368 |
| 355 | 330 |
| 356 | 361 |
| 357 | 334 |
| 358 | 334 |
| 359 | 330 |
| 360 | 387 |
| 361 | 384 |

The structures of the compounds of the invention are shown in the following Tables. These compounds can be easily prepared by the above preparation methods, methods described in Examples or Preparations, or methods that are well-known to one skilled in the art, or its variations.

Symbols in the Tables have the following meaning.

No: compound number
R: substituent group in the general formula
Me: methyl, Et: ethyl.

TABLE 3

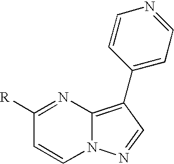

| No | R |
|---|---|
| A1 | 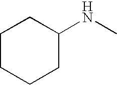 |
| A2 | 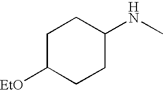 |
| A3 | 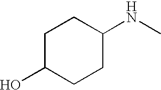 |
| A4 | 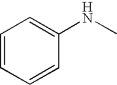 |
| A5 | 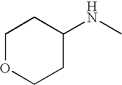 |
| A6 | 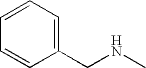 |
| A7 | 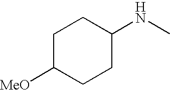 |
| A8 | 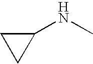 |
| A9 | 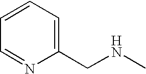 |
| A10 | 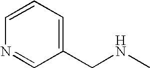 |
| A11 | 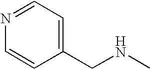 |
| A12 | 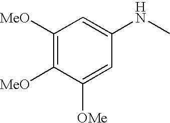 |
| A13 | 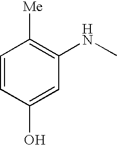 |
| A14 | 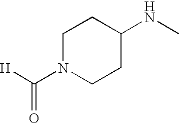 |
| A15 | 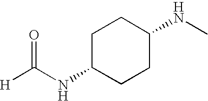 |
| A16 | 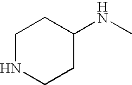 |
| A17 | 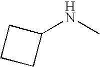 |
| A18 | 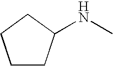 |
| A19 | 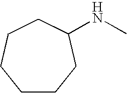 |
| A20 | 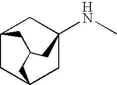 |
| A21 | 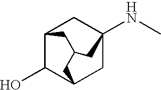 |
| A22 | 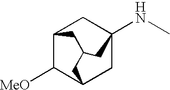 |
| A23 | 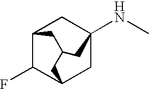 |
| A24 | 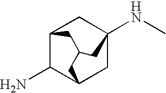 |
| A25 | 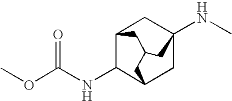 |
| A26 | 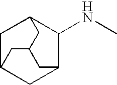 |
| A27 | 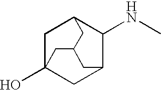 |
| A28 | 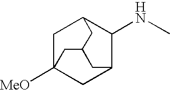 |

TABLE 4

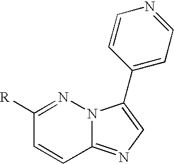

| No | R |
|---|---|
| B1 | 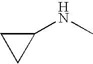 |
| B2 | 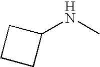 |
| B3 | 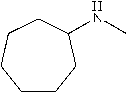 |
| B4 | 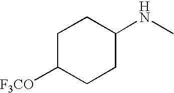 |
| B5 | 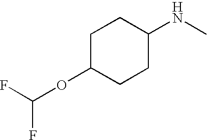 |
| B6 | 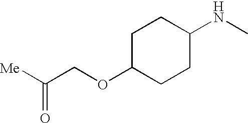 |
| B7 | 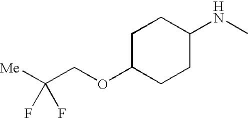 |
| B8 | 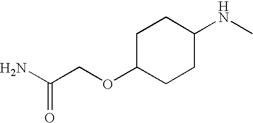 |
| B9 | 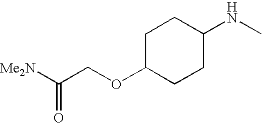 |
| B10 | 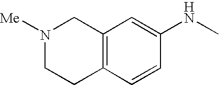 |
| B11 | 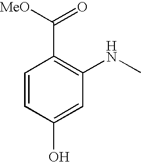 |
| B12 | 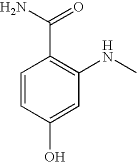 |
| B13 | 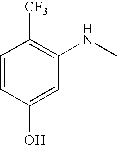 |
| B14 | 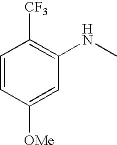 |
| B15 | 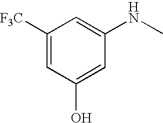 |
| B16 | 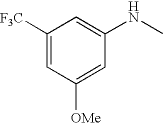 |
| B17 | 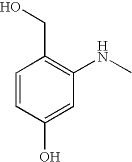 |
| B18 | 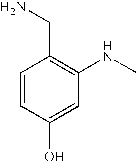 |
| B19 | 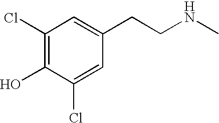 |
| B20 | 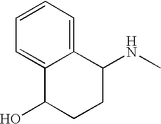 |
| B21 | 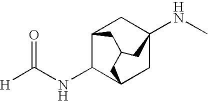 |
| B22 | 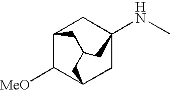 |
| B23 | 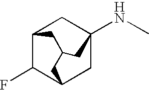 |
| B24 | 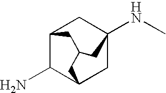 |
| B25 | 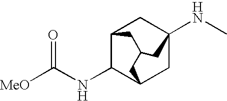 |
| B26 | 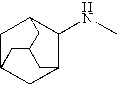 |
| B27 | 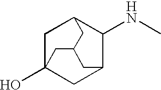 |
| B28 | 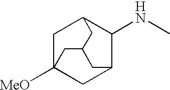 |
| B29 | 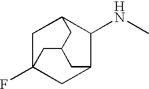 |
| B30 | 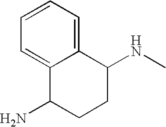 |
| B31 | 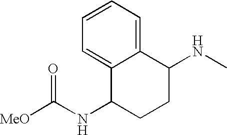 |
| B32 | 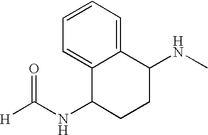 |
| B33 | 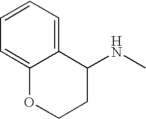 |
| B34 | 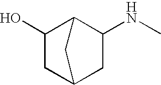 |
| B35 | 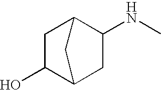 |
| B36 | 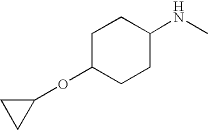 |
| B37 | 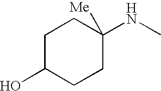 |
| B38 | 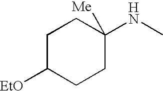 |
| B39 | 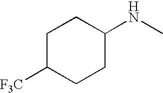 |
| B40 | 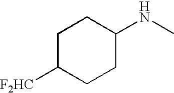 |

TABLE 5

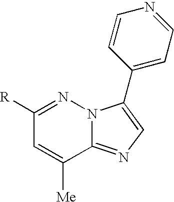

| No | R |
|---|---|
| C1 | 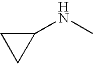 |
| C2 | 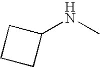 |
| C3 | 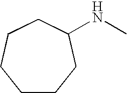 |
| C4 | 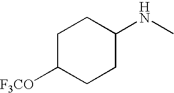 |
| C5 | 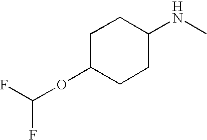 |
| C6 | 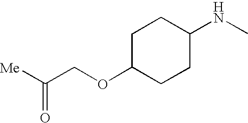 |
| C7 | 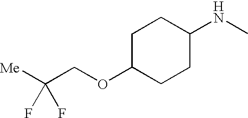 |
| C8 | 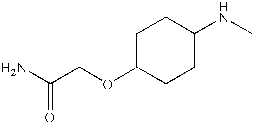 |
| C9 | 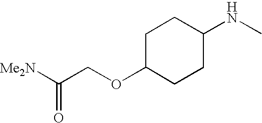 |
| C10 | 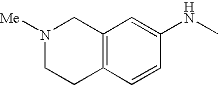 |
| C11 | 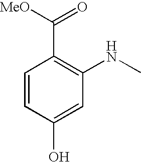 |
| C12 | 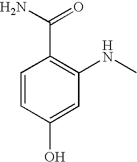 |
| C13 | 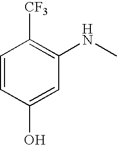 |
| C14 | 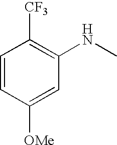 |
| C15 | 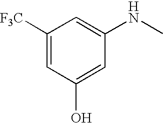 |
| C16 | 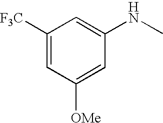 |
| C17 | 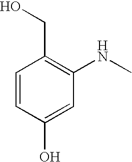 |
| C18 | 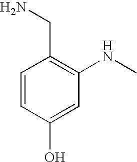 |
| C19 | 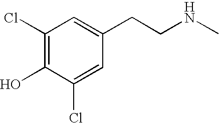 |
| C20 | 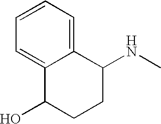 |
| C21 | 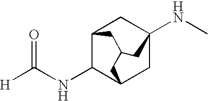 |
| C22 | 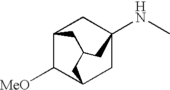 |
| C23 | 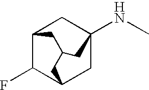 |
| C24 | 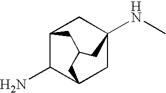 |
| C25 | 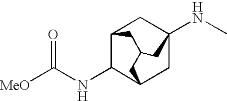 |
| C26 | 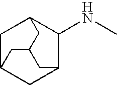 |
| C27 | 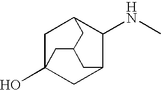 |
| C28 | 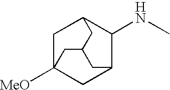 |
| C29 | 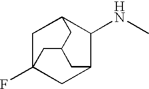 |
| C30 | 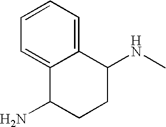 |
| C31 | 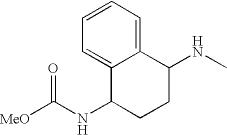 |
| C32 | 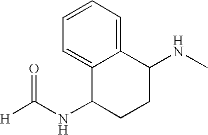 |
| C33 | 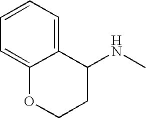 |
| C34 | 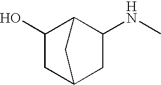 |
| C35 | 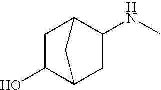 |
| C36 | 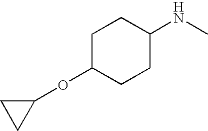 |
| C37 | 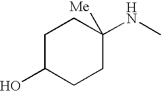 |
| C38 | 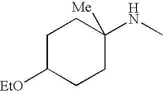 |
| C39 | 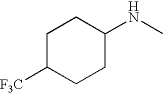 |
| C40 | 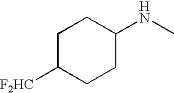 |

TABLE 6

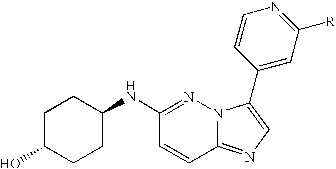

| No | R |
|---|---|
| D1 | 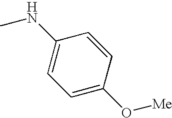 |
| D2 | 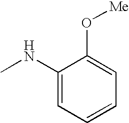 |
| D3 | 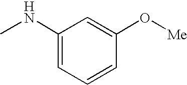 |
| D4 | 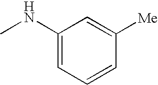 |
| D5 | 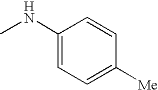 |
| D6 | 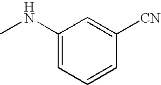 |
| D7 | 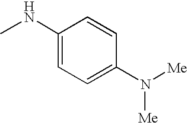 |
| D8 | 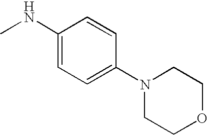 |
| D9 | 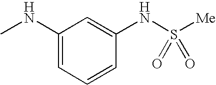 |
| D10 | 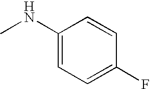 |
| D11 | 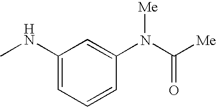 |
| D12 | 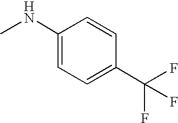 |
| D13 | 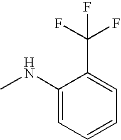 |
| D14 | 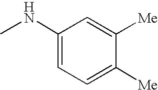 |
| D15 | 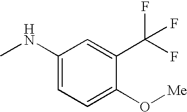 |
| D16 | 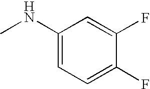 |
| D17 | 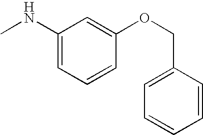 |
| D18 | 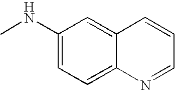 |
| D19 | 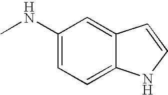 |
| D20 | 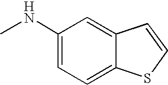 |
| D21 | 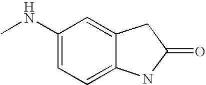 |
| D22 | 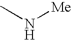 |
| D23 | 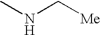 |
| D24 | 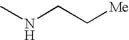 |
| D25 | 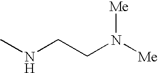 |
| D26 | 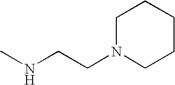 |
| D27 | 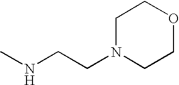 |
| D28 | 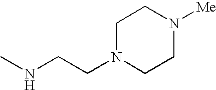 |
| D29 | 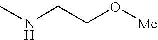 |
| D30 | 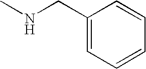 |
| D31 | 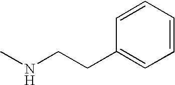 |
| D32 | 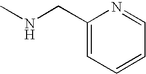 |
| D33 | 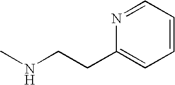 |
| D34 | 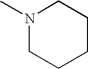 |
| D35 | 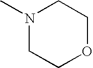 |
| D36 | 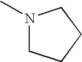 |
| D37 | 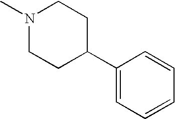 |
| D38 | 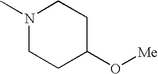 |
| D39 | 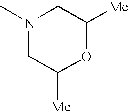 |
| D40 | 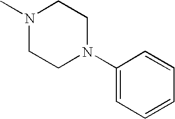 |
| D41 | 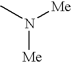 |
| D42 | 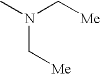 |
| D43 | 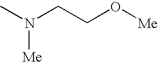 |
| D44 | 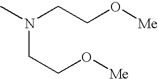 |
| D45 | 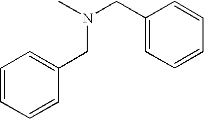 |
| D46 | 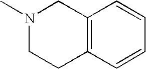 |
| D47 | 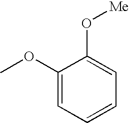 |
| D48 | 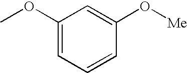 |
| D49 | 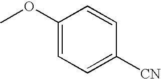 |
| D50 | 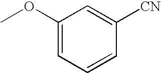 |
| D51 | 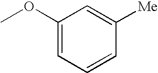 |
| D52 | 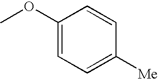 |
| D53 | 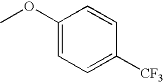 |
| D54 | 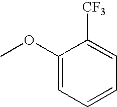 |
| D43 | 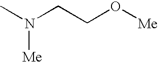 |
| D44 | 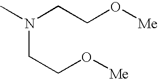 |
| D45 | 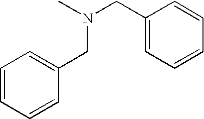 |
| D46 | 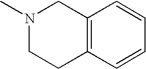 |
| D47 | 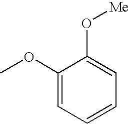 |
| D48 | 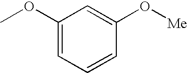 |
| D49 | 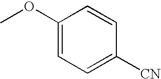 |
| D50 | 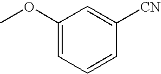 |
| D51 | 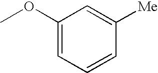 |
| D52 | 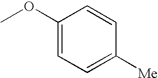 |
| D53 | 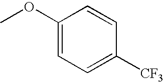 |
| D54 | 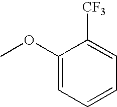 |
| D55 | 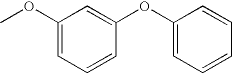 |
| D56 | 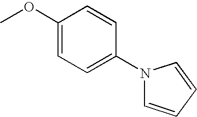 |
| D57 | 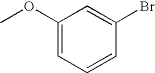 |
| D58 | 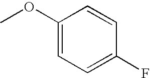 |
| D59 | 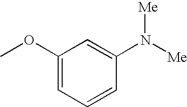 |
| D60 | 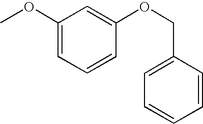 |
| D61 | 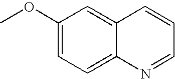 |
| D62 | 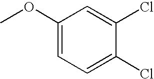 |
| D63 | 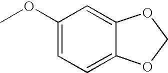 |
| D64 | 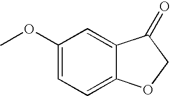 |
| D65 | 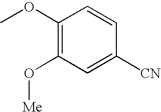 |
| D66 | 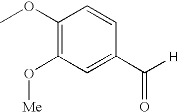 |
| D67 | 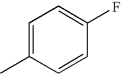 |
| D68 | 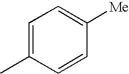 |
| D69 | 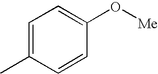 |
| D70 | 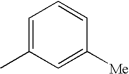 |
| D71 | 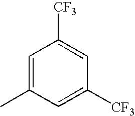 |
| D72 | 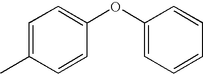 |
| D73 | 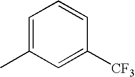 |
| D74 | 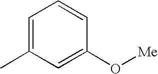 |
| D75 | 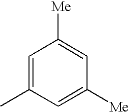 |
| D76 | 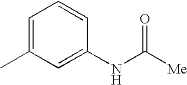 |
| D77 | 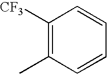 |
| D78 | 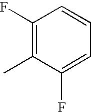 |
| D79 | 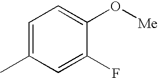 |
| D80 | 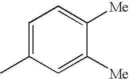 |
| D81 | 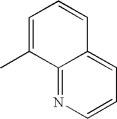 |
| D82 | 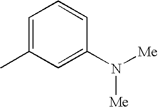 |
| D83 | 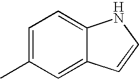 |
| D84 | 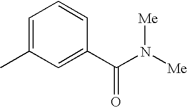 |
| D85 | 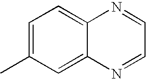 |
| D86 | 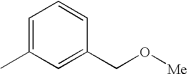 |
| D87 | 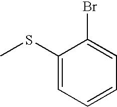 |
| D88 | 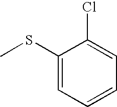 |
| D89 | 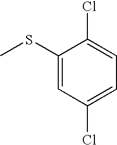 |
| D90 | 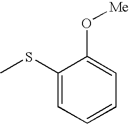 |
| D91 | 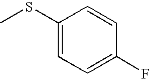 |

1. A fused heterocyclic compound represented by the formula (I): 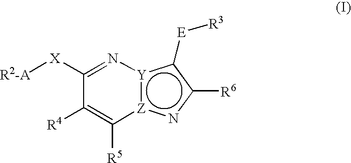
wherein
one of Y and Z is C atom, and the other is N atom,
—X— is bond, —N(R¹)—, —O—, —S—, —S(═O)—S(═O)₂—;
—R¹ is hydrogen or lower alkyl;
-A- is bond, lower alkylene or lower alkenylene, each of which may be substituted by one or more substituents selected from the group consisting of —OH and —NR¹¹R¹², wherein a methylene unit of -A- is optionally replaced by —O— or —C(═O)—;
—R¹¹ and —R¹² are the same or different, each being hydrogen or lower alkyl;
—R² is hydrogen, cycloalkyl, aryl, 5- or 6-membered non-aromatic heterocycle or 5- or 6-membered aromatic heterocycle, each of which may be substituted, or alternatively —R¹ and “-A-R²” taken with the adjacent nitrogen atom may form 5-, 6- or 7-membered cyclic amino, which may be substituted;
-E- represents bond, lower alkylene, lower alkenylene or lower alkynylene, wherein a methylene unit of -E- is optionally replaced by —O—, —(CO)O—, —NH—, —NHCO—, —NHSO₂— or —NH(CO)NH—;
—R³ is cycloalkyl, aryl, 5- or 6-membered non-aromatic heterocycle or 5- or 6-membered aromatic heterocycle, each of which may be substituted and may be fused with benzene; and
—R⁴, —R⁵ and —R⁶ are the same or different, each being hydrogen, halogen, lower alkyl, —O— lower alkyl or aryl;
provided that (i) when -A- is bond, —X— is NH, —R² is 4-tetrahydropyranyl and —R³ is 3-chlorophenyl, then Y is C atom and Z is N atom;
(ii) when —X— is NH, —R² is cyclopropyl, 2-pyridyl, 3-pyridyl, 2-thienyl or 4-fluorophenyl and —R³ is 3-acetylphenyl, 3-chlorophenyl, 4-chlorophenyl, phenyl, 2-furyl or 2-thienyl, then -A- is bond.
or a pharmaceutically acceptable salt thereof.

2. The compound of claim 1, wherein
one of Y and Z is C atom, and the other is N atom;
—X— is —N(R¹)—, —O—, or —S—;
—R¹ is hydrogen or lower alkyl;
-A- is bond, lower alkylene or lower alkenylene each of which may be substituted by one or more substituents selected from the group consisting of —OH and —NR¹¹R¹², wherein a methylene unit of -A- is optionally replaced by —O— or —C(═O)—;
—R¹¹ and —R¹² are the same or different, each being hydrogen or lower alkyl;
—R² is hydrogen, C₅₋₁₀ cycloalkyl, aryl, 5- or 6-membered non-aromatic heterocycle which contains one to three heteroatom(s) or 5- or 6-membered aromatic heterocycle which contains one heteroatom; each of which may be substituted with one to three substituent (s) selected from the group consisting of halogen, hydroxy, nitro, lower alkyl, —O-lower alkyl, —O-lower alkyl having halogen, —O-(6-membered cyclic amino), —CONH-lower alkyl, —C(O)NH-aryl, —S(O)₂—aryl, —C(O)O-lower alkyl, —C(O)OH, —C(O)NH—O-lower alkyl, —NR¹¹R¹², 6-membered non-aromatic heterocycle, and —O-(6-membered aromatic heterocycle), or alternatively —R¹ and “-A-R²” taken with the adjacent nitrogen atom may form 5-, 6- or 7-membered cyclic amino, which may be substituted;
-E- is bond, lower alkylene, lower alkenylene or lower alkynylene, wherein a methylene unit of -E- is optionally replaced by —NHSO₂— or —NH(CO)NH—;
—R³ is 5- or 6-membered non-aromatic heterocycle or 5- or 6-membered aromatic heterocycle which contains one to two nitrogen atom, which may be fused with benzene; each of which may be substituted with one to three substituent (s) selected from the group consisting of halogen, lower alkyl, lower alkyl having halogen, lower alkyl having hydroxyl, —OH, cyano, —O-lower alkyl, phenyl, —O-phenyl, —S-phenyl, —C(O)O-lower alkyl, —C(O)NH₂, —NHCO-aryl, —NHC(O)O-lower alkyl and —NR¹¹R¹²; and
—R⁴, —R⁵ and —R⁶ are the same or different, each being hydrogen, halogen, lower alkyl, —O-lower alkyl or aryl;
or a pharmaceutically acceptable salt thereof.

3. The compound of claim 2, wherein
one of Y and Z is C atom, and the other is N atom;
—X— is —N(R¹)—, or —O—;
—R¹ is hydrogen;
-A-, is bond or lower alkylene;
—R² is hydrogen, cyclohexyl, phenyl, adamantyl, pyridinyl, piperidinyl, or tetrahydropyranyl; each of which may be substituted with one to two substituent(s) selected from the group consisting of hydroxy, halogen, methyl and lower alkyloxy optionally substituted with halogen;
-E- is bond;
—R³ is pyridinyl which may be substituted with halogen;
—R⁴, —R⁵ and —R⁶ are the same or different, each being hydrogen, halogen, methyl, or phenyl;
or a pharmaceutically acceptable salt thereof.

4. The compound of claim 3, which is
(1) N-Cyclohexyl-3-(4-pyridinyl)-imidazo[1,2-b]pyridazin-6-amine
(2)-3-(4-Pyridinyl)-N-(tetrahydro-2H-pyran-4-yl) imidazo[1,2-b]pyridazin-6-amine
(3) N-Phenyl-3-(4-pyridinyl)-imidazo[1,2-b]pyridazin-6-amine
(4) N,3-Dipyridin-4-ylimidazo[1,2-b]pyridazin-6-amine
(5) N-Benzyl-3-(4-pyridinyl)-imidazo[1,2-b]pyridazin-6-amine
(6) N-Adamantan-1-yl-3-(4-pyridinyl)-imidazo[1,2-b]pyridazin-6-amine
(7) trans-4-{[3-(2-Chloropyridin-4-yl)imidazo[1,2-b]pyridazin-6-yl]amino}cyclohexanol
(8) N-(trans-4-Ethoxycyclohexyl)-3-pyridin-4-ylimidazo[1,2-b]pyridazin-6-amine
(9) (1R,2R,3S,5s)-5-{[3-(4-Pyridyl)imidazo[1,2-b]pyridazin-6-yl]amino}-2-adamantanol
(10) 4-Methyl-3-[(3-(4-pyridinyl)-imidazo[1,2-b]pyridazin-6-yl)amino]phenol
(11) trans-4-[(8-Methyl-3-(4-pyridinyl)-imidazo[1,2-b]pyridazin-6-yl)amino]cyclohexanol
(12) trans-4-{[3-(2-Bromopyridin-4-yl)imidazo[1,2-b]pyridazin-6-yl]amino}cyclohexanol
(13) N-(2,5-Dichlorobenzyl)-3-(4-pyridinyl)-imidazo[1,2-b]-pyridazin-6-amine
(14) N-[2-(Difluoromethoxy)benzyl]-3-pyridin-4-ylimidazo-[1,2-b]pyridazin-6-amine
(15) N-[3-Chloro-2-fluorobenzyl]-3-pyridin-4-ylimidazo-[1,2-b]pyridazin-6-amine or a pharmaceutically acceptable salt thereof.

5. A Lck inhibitor comprising the compound of claim 1.

6. A pharmaceutical composition for treating or preventing rejection reaction in organ transplantation, autoimmune diseases, asthma, atopic dermatitis, which comprises the compound of claim 1.

7. A pharmaceutical composition which comprises, as an active ingredient, a compound of claim 1 in admixture with pharmaceutically acceptable and substantially non-toxic carrier or excipient.

8. The compound of any of claim 1 for use as a medicament.

9. A method for inhibiting Lck, comprising using the compound of claim 1.

10. Use of the compound of claim 1 for the manufacture of a medicament for inhibiting Lck.

11. A method for treating or preventing rejection reaction in organ transplantation, autoimmune diseases, asthma, atopic dermatitis, which comprises administering an effective amount of the compound of claim 1 to a human being or an animal.

12. Use of the compound of claim 1 for the manufacture of a medicament for treating or preventing rejection reaction in organ transplantation, autoimmune diseases, asthma, atopic dermatitis.

13. A commercial package comprising the pharmaceutical composition of claim 6 or claim 7 and a written matter associated therewith, the written matter stating that the pharmaceutical composition may or should be used for treating or preventing rejection reaction in organ transplantation, autoimmune diseases, asthma, atopic dermatitis.
